(19) United States
(12) Patent Application Publication
(10) Pub. No.: US 2005/0192286 A1
(43) Pub. Date: Sep. 1, 2005

(54) Ligands of melanocortin receptors and compositions and methods related thereto

(75) Inventors: Joe Anh Tran, San Marcos, CA (US); Fabio C. Tucci, San Diego, CA (US); Wanlong Jiang, San Diego, CA (US); Wei-Chuan C. Chen, San Diego, CA (US); Dragan Marinkovic, Del Mar, CA (US); Melissa Arellano, San Diego, CA (US); Nicole White, Aliso Viejo, CA (US); Chen Chen, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Neurocrine Biosciences, Inc., San Diego, CA (US)

(21) Appl. No.: 10/972,064

(22) Filed: Oct. 22, 2004

Related U.S. Application Data
(60) Provisional application No. 60/513,626, filed on Oct. 22, 2003.

(51) Int. Cl. A61K 31/496; C07D 43/02

(52) U.S. Cl. 514/254.05; 544/366

(57) ABSTRACT

Compounds which function as melanocortin receptor ligands and having utility in the treatment of melanocortin receptor-based disorders. The compounds have the following structure (I): 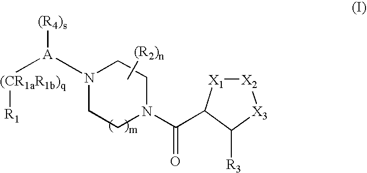
including stereoisomers, prodrugs, and pharmaceutically acceptable salts thereof, wherein m, n, q, s, R₁, R₁a, R_{1b}, R₂, R₃, R₄, X₁, X₂ and X₃ are as defined herein. Pharmaceutical compositions containing a compound of structure (I), as well as methods relating to the use thereof, are also disclosed.

### CROSS-REFERENCE TO RELATED APPLICATION

This application claims priority to U.S. Provisional Application Ser. No. 60/513,626, filed Oct. 22, 2003, the entire disclosure of which is incorporated by reference herein.

### BACKGROUND OF THE INVENTION

1. Field of the Invention

This invention is generally directed to ligands of a melanocortin receptor, as well as to compositions and methods for using such ligands to alter activity of a melanocortin receptor.

2. Description of the Prior Art

Melanocortin (MC) receptors are members of the family of G-protein coupled receptors. To date, five distinct MC receptors (i.e., MC1-R, MC2-R, MC3-R, MC4-R and MC5-R) have been identified in a variety of tissues and these receptors have been shown to mediate a number of physiological processes. Ligands, including peptides and small molecules, have been shown to act as agonists or antagonists at these receptors.

The role of specific MC receptors in physiological processes has been the object of intense study since their discovery and cloning. These receptors are expressed in a variety of tissues including melanocytes, adrenal cortex, brain, gut, placenta, skeletal muscle, lung, spleen, thymus, bone marrow, pituitary, gonads and adipose tissue. A putative role of MC receptors has been shown in melanocytes, stimulatory actions on learning, attention and memory, motor effects, modification of sexual behavior, facilitation of nerve regeneration, anti-inflammatory and antipyretic effects, and the regulation of food intake and body weight.

The pro-opiomelanocortin (POMC) gene product is processed to produce a number of biologically active peptides that are expressed in the pituitary, and two locations in the brain: the arcuate nucleus of the hypothalamus and the solitary tract nucleus of the brain stem. These peptides elicit a range of biological activities. Two POMC peptides, α-melanocyte stimulating hormone (α-MSH) and adrenocorticotropic hormone (ACTH), control melanocyte and adrenocortical function, respectively, in the periphery.

Cloning studies have defined a family of five melanocortin (MC) receptors that respond to POMC peptides (reviewed in Rec. Prog. Hor. Res. 51:287-318, 1996). Each receptor in this family is pharmacologically distinct in its particular response to the POMC peptides α-MSH, γ-MSH and ACTH and to two peptide antagonists. MC4-R differs from the other MC receptors in that it binds both natural melanocortin antagonists, agouti (Nature 371:799-802, 1994) and agouti-related protein (AgRP) (Biochem. Biophys. Res. Commun. 237:629-631, 1997). In contrast, MC1-R only binds agouti, MC2-R does not bind AgRP, MC3-R only binds AgRP, and MC5-R has only low affinity binding for AgRP (Mol. Endocrinology 13:148-155, 1999).

The expression of specific MC receptors is restricted anatomically. MC1-R is expressed primarily in melanocytes, while MC2-R is expressed in adrenocortical cells. MC3-R is expressed in brain, placenta and gut, and MC4-R is expressed primarily in the brain where its mRNA can be detected in nuclei that bind α-MSH. MC4-R is notably absent from adrenal cortex, melanocyte and placental tissues. Both MC3-R and MC4-R are expressed in arcuate and paraventricular neurons. MC5-R is expressed in brain, adipose tissues, muscle and exocrine glands.

α-Melanocyte stimulating hormone (α-MSH) is a tridecapeptide whose principal action (i.e., the activation of a set of G-protein coupled melanocortin receptors), results in a range of physiological responses including pigmentation, sebum production and feeding behavior. Cyclized peptide derivatives of α-MSH are potent modulators of these receptors. When administered by intracerebroventricular (i.c.v) injection into fasted animals, peptides exhibiting MCR-4 antagonist activity increase food intake and body weight. Moreover, overexpression of a naturally occurring peptide antagonist, agouti-related peptide (AgRP) has a similar effect on food intake and body weight. The development of small molecule antagonists of the MC4-R would selectively enhance the feeding response. MC4-R antagonists have a unique clinical potential because such compounds would stimulate appetite as well as decrease metabolic rate. Additionally, chronic MC4-R blockade causes an increase in lean body mass as well as fat mass, and the increase in lean body mass is independent of the increase in fat mass. Orally active forms of a small molecule MC4-R antagonist would provide a therapeutic strategy for indications in which cachexia is a symptom.

The MC receptors are also key mediators of steroid production in response to stress (MC2-R), regulation of weight homeostasis (MC4-R), and regulation of hair and skin pigmentation (MC1-R). They may have additional applications in controlling both insulin regulation (MC4-R) and regulation of exocrine gland function (MC5-R) (Cell 91:789-798, 1997); the latter having potential applications in the treatment of disorders such as acne, dry eye syndrome and blepharitis. Melanocortin peptides have also been reported to have anti-inflammatory activity, although the receptor(s) involved in mediating these effects have not yet been determined. Endocrine disorders such as Cushing's disease and congenital adrenal hyperplasia, which are characterized by elevated levels of ACTH, could be effectively treated with ACTH receptor (MC2-R) antagonists. Some evidence suggests that depression, which is characterized by elevated levels of glucocorticoids, may also be responsive to these same compounds. Similarly, elevated glucocorticoids can be an etiological factor in obesity. Synthetic melanocortin receptor agonists have been shown to initiate erections in men (J. Urol. 160:389-393, 1998). An appropriate MC receptor agonist could be an effective treatment for certain sexual disorders.

MC1-R provides an ideal target for developing drugs that alter skin pigmentation. MC1-R expression is localized to melanocytes where it regulates eumelanin pigment synthesis. Two small clinical trials indicate that broad-spectrum melanocortin agonists induce pigmentation with limited side effects. The desired compound would have a short half-life and be topically applied. Applications include skin cancer prevention, UV-free tanning, inhibition of tanning and treatment of pigmentation disorders, such as tyrosinase-positive albinism.

The role of melanocortin receptors in regulation of adiposity signaling and food intake has been recently reviewed (Nature 404:661-669, 2000). Direct experimental evidence for the individual role of MC4 and MC3 receptors in energy homeostasis has not yet been reported due to the lack of potent and specific MC4 and MC3 agonists. Central administration of synthetic, non-selective MC-3R and MC4-R agonists, such as cyclic side-chain-lactan-modified peptide MT-II suppresses food intake in rodents and monkeys, and stimulates energy expenditure resulting inreduced adiposity (Endocrinology 142:2586-2592, 2001). Conversely, selective peptide antagonists of the MC4 receptor stimulate food consumption and result in increased body weight, suggesting the main effects of agonist induced inhibition of food consumption are mediated by MC4-R receptor activity. (European J. Pharmacol. 405:25-32, 2000). Selective small molecule MC4-R antagonists also stimulate food intake in animal models of cachexia.

Genetically modified animals lacking the MC4-R receptor are hyperphagic and obese (Cell 88:131-141, 1997). Humans with defective melanocortin 4 receptors exhibit marked hyperphagia and increased body mass relative to their normal siblings (Nature Genet. 20:111-114, 1998). In addition, studies with mice lacking functional MC-3 receptors suggest that agonist stimulation of this receptor may also play a role in control of energy homeostasis, feeding efficiency, metabolism and bodyweight (Endocrinology 141:3518-3521, 2000). Therefore MC4-R and MC3-R agonists may be useful in the control of obesity and in treatment of related disorders including diabetes.

Due to their important biological role, a number of agonists and antagonists of the MC receptors have been suggested. For example, U.S. Pat. No. 6,054,556 is directed to a family of cyclic heptapeptides which act as antagonists for MC1, MC3, MC4 and MC5 receptors; U.S. Pat. No. 6,127,381 is directed to isoquinoline compounds which act upon MC receptors for controlling cytokine-regulated physiologic processes and pathologies; and published PCT Application No. WO 00/74679 is directed to substituted piperidine compounds that act as selective agonists of MC4-R. Published PCT Application No. WO01/05401 is directed to small peptides that are MC3-R specific agonists. Recent PCT publications WO02/059095, WO02/059107, WO02/059108, WO02/059117, WO03/009847 and WO03/009850 describe melanocortin receptor agonists which may be useful for the treatment of obesity, among other diseases. WO03/031410 and WO03/068738 describe certain compounds which act at melanocortin receptor(s).

Accordingly, while significant advances have been made in this field, there is still a need in the art for ligands to the MC receptors and, more specifically, to agonists and/or antagonists to such receptors, particularly small molecules. There is also a need for pharmaceutical compositions containing the same, as well as methods relating to the use thereof to treat conditions associated with the MC receptors. The present invention fulfills these needs, and provides other related advantages.

### BRIEF SUMMARY OF THE INVENTION

In brief, this invention is generally directed to compounds that can function as melanocortin (MC) receptor ligands. In this context, “ligands” are molecules that bind or form a complex with one or more of the MC receptors. This invention is also directed to compositions containing one or more compounds in combination with one or more pharmaceutically acceptable carriers, as well as to methods for treating conditions or disorders associated with MC receptors.

In one embodiment, this invention is directed to compounds which have the following structure (I): 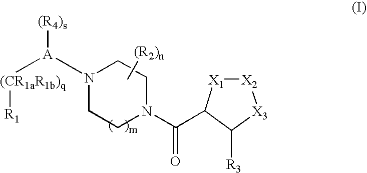
including pharmaceutically acceptable salts, esters, solvates, stereoisomers, and prodrugs thereof, wherein m, n, q, s, R₁, R₁ₐ, R_{1b}, R₂, R₃, R₄, X₁, X₂ and X₃ are as defined herein.

The compounds of this invention may have utility over a broad range of therapeutic applications, and may be used to treat disorders or illnesses, including (but not limited to) eating disorders, obesity, inflammation, pain, chronic pain, skin disorders, skin and hair coloration, sexual dysfunction, dry eye, acne, anxiety, depression, and/or Cushing's disease. A representative method of treating such a disorder or illness includes administering a pharmaceutically effective amount of a compound of this invention, typically in the form of a pharmaceutical composition, to an animal (also referred to herein as a “patient”, including a human) in need thereof. The compound may be an antagonist or agonist or may stimulate a specific melanocortin receptor while functionally blocking a different melanocortin receptor. Accordingly, in another embodiment, pharmaceutical compositions are disclosed containing one or more ligands of this invention in combination with a pharmaceutically acceptable carrier.

In one embodiment, compounds of the present invention may be agonists to one or more MC receptors, and may be useful in medical conditions where a melanocortin receptor agonist is beneficial. For example, the compounds may be utilized as MC4 receptor specific agonists or MC3 receptor specific agonists. Alternatively, the compounds may have mixed activity on the MC3 receptor and MC4 receptor, and may even function as an agonist to one receptor and an antagonist to the other. In this context, the compounds may be used to treat obesity, erectile and/or sexual dysfunction, or diabetes mellitus.

In another embodiment, the compounds may serve as antagonists to either the MC3 receptor or MC4 receptor. Such antagonists may have beneficial therapeutic effects, especially in the treatment of cachexia or wasting disease associated with cancer, AIDS, failure to thrive syndrome, and diseases associated with aging and senility. In more specific embodiments, the compounds may be MC4 receptor specific antagonists for treatment of cachexia or wasting disease associated with cancer, AIDS, failure to thrive syndrome, and diseases associated with aging and senility.

These and other aspects of this invention will be apparent upon reference to the following detailed description and attached figures. To that end, certain patent and other documents are cited herein to more specifically set forth various aspects of this invention. Each of these documents is hereby incorporated by reference in its entirety.

### DETAILED DESCRIPTION OF THE INVENTION

As mentioned above, in one embodiment the present invention is generally directed to compounds having the following structure (I): 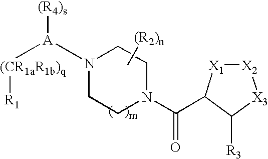
and pharmaceutically acceptable salts, esters, solvates, stereoisomers, and prodrugs thereof,
wherein:
A is a C₅₋₇cycloalkyl, aryl, or heteroaryl;
X₁ is —CR₅R₆—, —NR₇—, —O—, or —C(═O)—;
X₂ and X₃ are the same or different and independently —CR₅R₆—, —NR₈—, —O—, or —C(═O)—;
or X₁ taken together with X₂ is —N═C(R₅)— or —C(R₅)═N—;
or X₂ taken together with X₃ is —N═C(R₅)— or —C(R₅)═N—;
R₁ is —(Y₁—Y₂)—NR₉R₁₀, —NR₈C(═O)R₁₁, —NR₈S(O)ₚR₁₂, —NR₈C(═O)R₁₃, imidazolyl, triazolyl, oxazolyl, or thiazolyl;
Y₁ is a direct bond, —O—, —S— —NR₈—, —C(═O)—, —C(═O)O—, —OC(═O)—, —NR₈C(═O)O—, —NR₈C(═O)—, —C(═O)NR₈—, —NR₈S(═O)ₚ—, —S(═O)ₚ—, —S(═O)ₚNR₈—, or —NR₈C(═O)NR₈—;
Y₂ is —(CR_{1c}R_{1d})ᵣ—;
R₁ₐ, R_{1b}, R_{1c}, and R_{1d} are at each occurrence the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
R₂ is at each occurrence the same or different and independently alkyl or substituted alkyl;
R₃ is aryl, substituted aryl, heteroaryl or substituted heteroaryl;
R₄ is at each occurrence the same or difference and independently hydroxy, halogen, cyano, nitro, alkyl, haloalkyl, substituted alkyl, aryl, substituted aryl, heterocycle, or substituted heterocycle;
R₅ and R₆ are the same or different and at each occurrence independently hydrogen, hydroxy, halogen, cyano, nitro, NR₉R₁₀, alkyl, substituted alkyl, aryl, substituted aryl, heterocycle, or substituted heterocycle;
R₇ is hydrogen, alkyl, substituted alkyl, —C(═O)R₁₁, or —SO₂R₁₂;
R₈ is at each occurrence the same or different and independently hydrogen, alkyl, substituted alkyl, heterocycle, substituted heterocycle, arylalkyl, substituted arylalkyl, heterocyclealkyl, substituted heterocyclealkyl, —C(═O)R₁₁, or —SO₂R₁₂;
R₉ and R₁₀ are the same or different and at each occurrence independently hydrogen, alkyl, substituted alkyl, aryl, substituted aryl, arylalkyl, substituted arylalkyl, heterocycle, substituted heterocycle, heterocyclealkyl, or substituted heterocyclealkyl,
or R₉ and R₁₀ taken together with the nitrogen atom to which they are attached form a heterocyclic ring or a substituted heterocyclic ring;
R₁₁, R₁₂ and R₁₃ are the same or different and independently hydrogen, alkyl, substituted alkyl, heterocycle, substituted heterocycle, aryl, substituted aryl, heterocyclealkyl, substituted heterocyclealkyl, arylalkyl or substituted arylalkyl;
m, p and s are independently 0, 1 or 2; and
n, q and r are independently 0, 1, 2, 3 or 4.

As used herein, the above terms have the following meaning:

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂cyclohexyl, and the like; while unsaturated cyclic alkyls include cyclopentenyl, cyclohexenyl, —CH₂cyclohexenyl, and the like. Cyclic alkyls are also referred to herein as a “homocycle”, and include bicyclic rings in which a homocycle is fused to a benzene ring. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-i -butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

A C₅₋₇cycloalkyl is cyclopentyl, cyclohexyl or cycloheptyl.

“Aryl” means an aromatic carbocyclic moiety such as phenyl or naphthyl.

“Arylalkyl” means an alkyl having at least one alkyl hydrogen atom replaced with an aryl moiety, such as benzyl (i.e., —CH₂phenyl), —(CH₂)₂phenyl, —(CH₂)₃phenyl, —CH(phenyl)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, oxadiazolyl, benzoxadiazolyl, thiadiazolyl, indazolyl and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4- to 7-membered monocyclic, or 7- to 10-membered bicyclic, heterocyclic ring which is saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂morpholinyl, and the like.

The term “substituted” as used herein means any of the above groups (i.e., alkyl, aryl, arylalkyl, heteroaryl, heteroarylalkyl, heterocycle and heterocyclealkyl) wherein at least one hydrogen atom is replaced with a substituent. In the case of an oxo substituent (“═O”) two hydrogen atoms are replaced. When substituted, “substituents” within the context of this invention include oxo, halogen, hydroxy, cyano, nitro, amino, alkylamino, dialkylamino, alkyl, alkoxy, thioalkyl, haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐR_{b}, —NRₐC(═O)OR_{b} —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, OS(═O)₂Rₐ, S(═O)₂ORₐ, —CH₂S(═O)₂Rₐ, —CH₂S(═O)₂NRₐR_{b}, ═NS(═O)₂Rₐ, and —S(═O)₂NRₐR_{b}, wherein Rₐ and R_{b} are the same or different and independently hydrogen, alkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, carbocycle, substituted carbocycle, carbocyclealkyl or substituted carbocyclealkyl.

“Halogen” means fluoro, chloro, bromo and iodo.

“Haloalkyl” means an alkyl having at least one hydrogen atom replaced with halogen, such as trifluoromethyl and the like.

“Alkoxy” means an alkyl moiety attached through an oxygen bridge (i.e., —O-alkyl) such as methoxy, ethoxy, and the like.

“Thioalkyl” means an alkyl moiety attached through a sulfur bridge (i.e., —S-alkyl) such as methylthio, ethylthio, and the like.

“Alkylamino” and “dialkylamino” mean one or two alkyl moiety attached through a nitrogen bridge (i.e., —N-alkyl) such as methylamino, ethylamino, dimethylamino, diethylamino, and the like.

In certain embodiments of structure (I), compounds of this invention have structure (II) when A is a C₅₋₇cycloalkyl, have structure (III) when A is aryl, and have structure (IV) when A is heteroaryl:

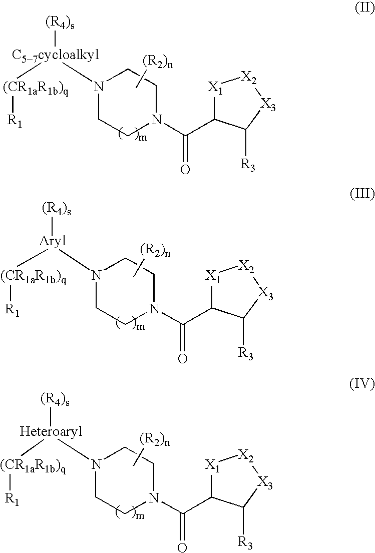

In the above structures (II), (III) and (IV) (as well as in structure (I) above), the “(R₄)s” moiety represents 0, 1 or 2 “R₄” substituents on the C₅₋₇cycloalkyl of structure (II), on the aryl moiety of structure (III), or on the heteroaryl moiety of structure (IV). When two R₄ substituents are present, they may be the same or different. Similarly, the “(R₂)ₙ” moiety represents 0, 1, 2, 3 or 4 “R₂” substituents on the pyrrolidine ring of structures (II), (III) and (IV) (as well as on structure (I) above).

In other embodiments of structure (I), compounds of this invention have structure (V) when R₁ is —(Y₁—Y₂)—NR₉R₁₀, have structure (VI) when R₁ is —NR₈C(═O)R₁₁, have structure (VII) when R₁ is —NR₈S(O)ₚR₁₂, have structure (VIII) when R₁ is —NR₈C(═O)OR₁₃, and have structures (IX), (X) (XI) and (XII) when R₁ is imidazolyl, triazolyl, oxazolyl and thiazolyl, respectively.

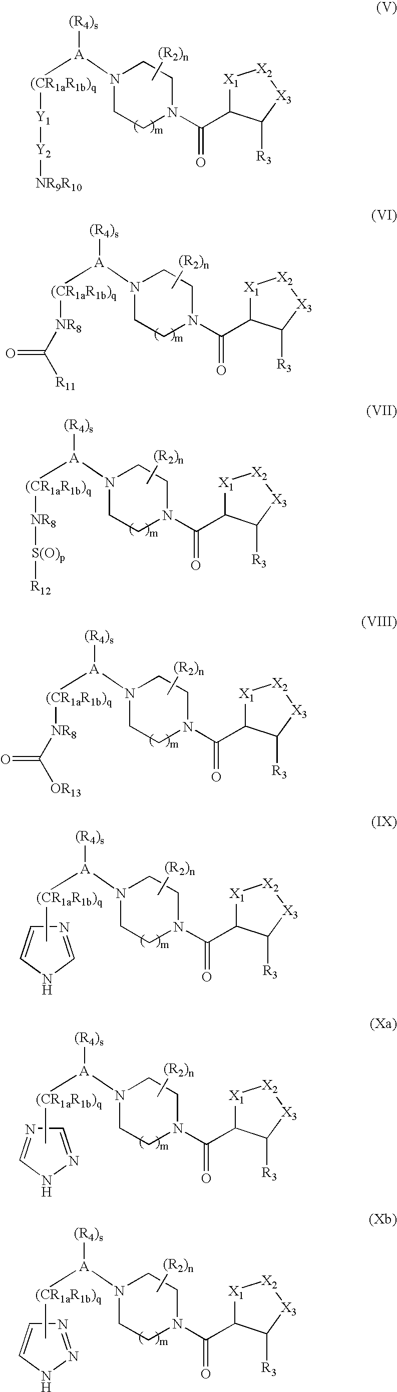

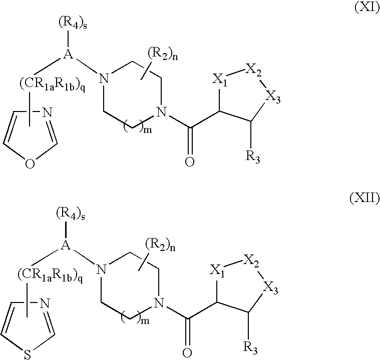

In a more specific embodiments of structure (V), compounds of this invention have structure (Va) when Y₁ is a direct bond and r is 0, and have structure (Vb) when Y₁ is —C(═O)— and r is 0:

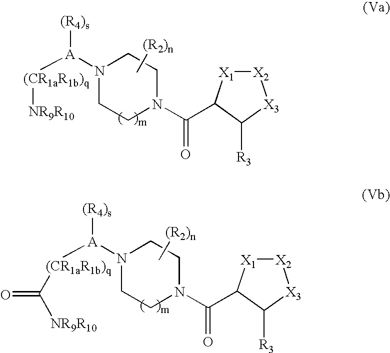

Further, in more specific embodiments of structure (IX), compounds of this invention have structure (IXa) when R₁ is 1-imidazolyl, and have structure (IXb) when R₁ is 2-imidazolyl.

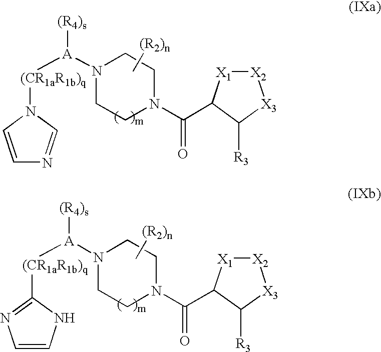

Further representative embodiments of R₁ include (but are not limited to) the following: —NR₉R₁₀, —C(═O)NR₉R₁₀, —OC(═O)NR₉R₁₀, —NR₈C(═O)NR₉R₁₀, —NR₈C(═O)R₁₁, —NR₈S(═O)ₚR₁₂, —R₈C(═O)OR₁₃, —S(═O)ₚNR₉R₁₀, —NR₈S(═O)ₚNR₉R₁₀, —O—(CR_{1c}R_{1d})ᵣNR₉R₁₀, —S—(CR_{1c}R_{1d})ᵣNR₉R₁₀, —C(═O)—(CR_{1c}R_{1d})ᵣNR₉R₁₀, —S(═O)ₚ—(CR_{1c}R_{1d})ᵣNR₉R₁₀, —C(═O)O—(CR_{1c}R_{1d})ᵣNR₉R₁₀, —NR₈—C(═O)—(CR_{1c}R_{1d})ᵣNR₉R₁₀, —C(═O)—NR₈—(CR_{1c}R_{1d})ᵣNR₉R₁₀, —OC(═O)O—(CR_{1c}R_{1d})ᵣNR₉R₁₀, —NR₈—C(═O)O—(CR_{1c}R_{1d})ᵣNR₉R₁₀, —NR₈—C(═O)—NR₈—(CR_{1c}R_{1d})ᵣNR₉R₁₀, and —NR₈—(CR_{1c}R_{1d})ᵣNR₉R₁₀.

In additional embodiments of structure (I), X₁, X₂ and X₃ taken together as “—X₁—X₂—X₃—” is —(CR₅R₆)₃—, —O—CR₅R₆—CR₅R₆—, —CR₅R₆—O—CR₅R₆—O—, —O—C(═O)—CR₅R₆—, —CR₅R₆—C(═O)—O—, —NR₇—CR₅R₆—CR₅R₆—, —CR₅R₆—NR₈—CR₅R₆—, —CR₅R₆—CR₅R₆—NR₈—, —NR₇—C(═O)—CR₅R₆, —CR₅R₆—C(═O)—NR₈—, —O—NR₈—CR₅R₆—O—NR₈—, —O—N═CR₅—, —NR₇—NR₈—CR₅R₆—, —CR₅R₆—NR₈—NR₈—, —NR₇—N═CR₅—, —O—CR₅R₆—NR₈—, —O—CR₅R₆—O—, —NR₇—C(═O)—O—, —NR₇—C(═O)—NR₈—, —N═CR₅—O—, —N═CR₅—NR₈— or —NR₇—O—CR₅R₆—, —CR₅R₆—NR₈—C(O)—, —O—CR₅═N—, —O—C(O)—NR₈—, —CR₅R₆—NR₈—O—, or —CR₅═—O—.

The compounds of the present invention may be prepared by known organic synthesis techniques, including the methods described in more detail in the following Reaction Schemes and Examples. Piperazine subunits of this invention are commercially available, are known in the literature, and/or may be synthesized from extensions of known methods. Furthermore, compounds of the present invention may be synthesized by a number of methods, both convergent and sequential, utilizing solution or solid phase chemistry.

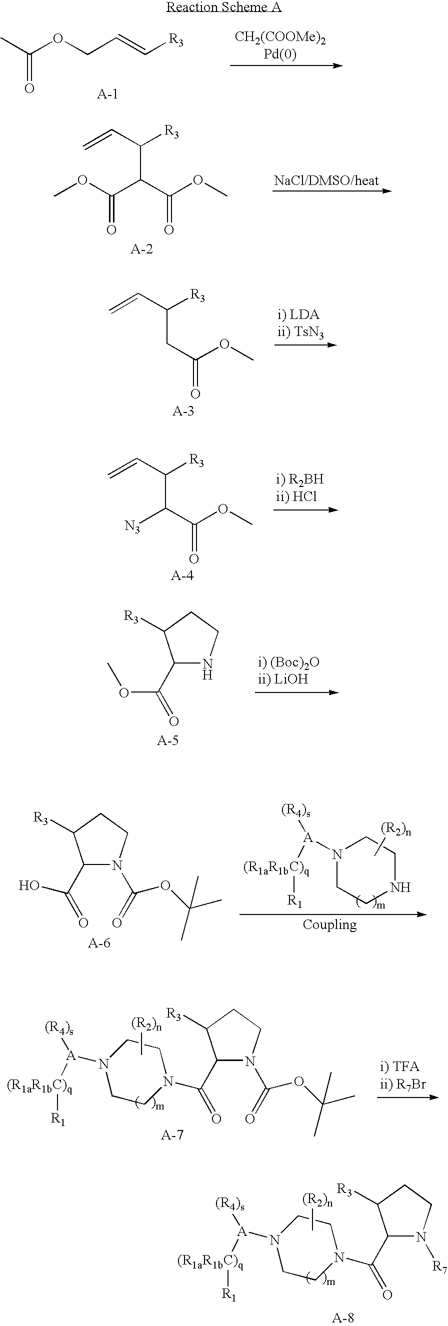

Palladium catalyzed coupling of allyl acetate (A-1) with malonate in a solvent such as THF, in the presence of a base such as potassium carbonate, gives the alkylated malonate A-2. A-2 may be decarboxylated in DMSO in the presence of sodium chloride at an elevated temperature (120-200° C.) to give the desired ester A-3. Introduction of an azide at the alpha-position of the ester A-3 is achieved by deprotonation with a strong base such as LDA and then quenching the reaction mixture with tosylate azide in a solvent such as THF at a temperature in the approximate range of −78 to −50° C. to give compound A-4. Reduction of the azide and hydroboration can be achieved by using a borane reagent such as dicyclohexylborane to give the pyrrolidine A-5 after acid (such as HCl) treatment. This pyrrolidine is then protected with a Boc-group and hydrolyzed under basic conditions such as lithium hydroxide to the corresponding acid A-6. Coupling of A-6 with a 4-substituted piperazine with a standard coupling protocol, such as EDC in DMF, gives the amide A-7, which could be further modified by deprotection of the Boc-group with TFA and then alkylated with alkyl halide in the presence of a base such as sodium bicarbonate to give compound A-8.

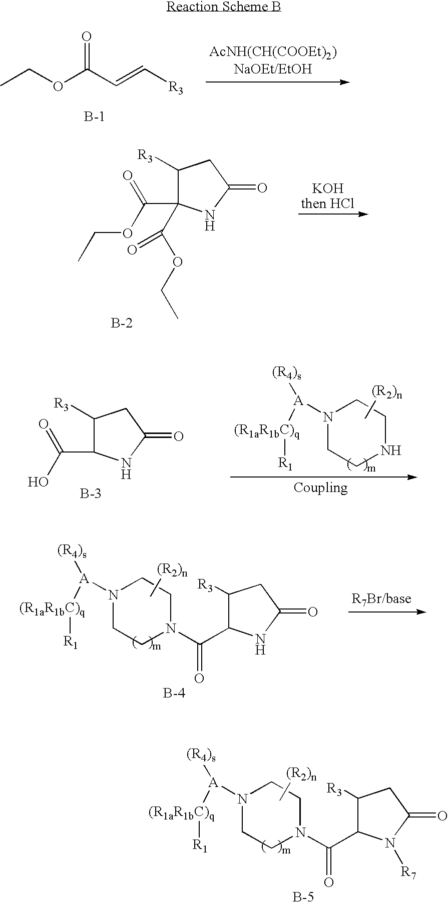

Ethyl cinnamate B-1 is condensed with acetamidomalonate under basic conditions (NaOEt) to give the intermediate B-2, which is hydrolyzed in aqueous potassium hydroxide, followed by treatment with acid to decarboxylate, to give the pyrrolidinone B-3. This compound may then be coupled with 4-substituted piperazine to give the amide B-4, which can be further modified by alkylation to give compound B-5.

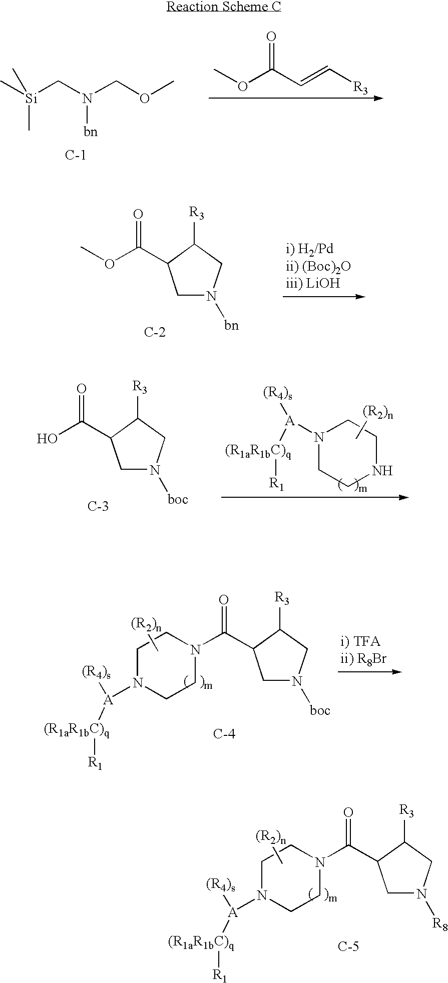

The aminomethylsilane C-1 is cyclized with (un)substituted cinnamate in the absence or presence of a base such as triethylamine in an inert solvent such as toluene or THF at a temperature of 0-100° C. to give the pyrrolidine C-2. The N-protecting group of C-2 may optionally be switched to a tert-butoxycarbonyl moiety by hydrogenation catalyzed by palladium, followed by reaction of the secondary amine with Boc₂O under basic conditions. Aqueous hydrolysis with a base such as LiOH affords the acid C-3, which is coupled with 4-substituted piperazine under standard conditions to give the amide C-4. This compound may be further modified to C-5 by deprotection of the Boc-group with TFA or HCl, followed by alkylation, acylation or sulfonylation to give the corresponding tertiary amine, amide, carbamide, urea, or sulfonamide.

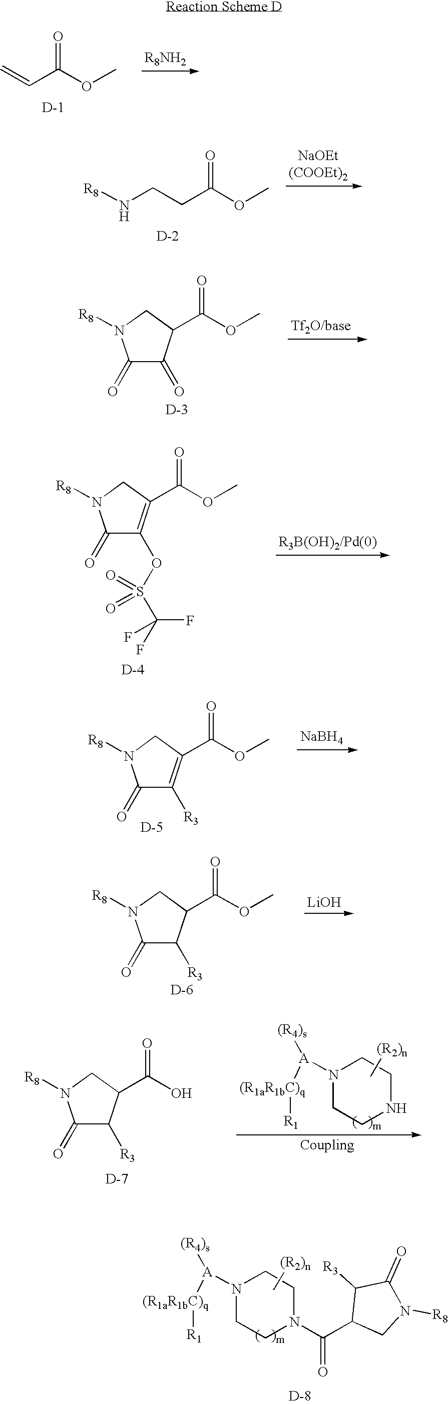

Beta-amino ester (D-2), synthesized from Michael addition of a primary amine with acrylate D-1, is cyclized to pyrrolidine-dione D-3 in the presence of oxylate and a base such as sodium ethoxide at −100° C. D-3 is converted to the corresponding triflate D-4 by treatment with triflic anhydride in the presence of a base such as triethylamine. Palladium-catalyzed coupling of D-4 with an appropriate boronic acid offers the compound D-5, which is reduced with a reducing agent such as sodium borohydride in a protic solvent such as methanol at ambient temperature to give pyrrolidinone D-6. Hydrolysis of the ester D-6 with a base such as LiOH in an aqueous media such as aqueous ethanol results in the corresponding acid D-7, which is coupled with the 4-substituted piperazine to give the desired pyrrolidinone D-8.

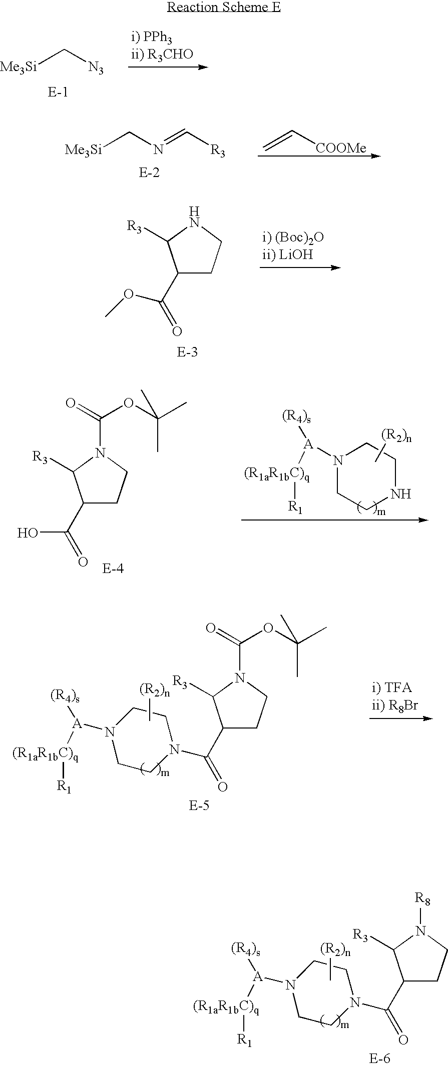

Trimethylsilylmethyl arylimine E-2, which may be obtained from an aza-Wittig reaction with aldehyde, is cyclized with acrylate to give the pyrrolidine E-3. Compound E-3 is then protected with Boc-group and is hydrolyzed under basic conditions to give the acid E-4. This compound is then coupled with the 4-substituted piperazine to offer the amide E-5, which can be further modified by deprotection, followed by an alkylation reaction to give the final compound E-6.

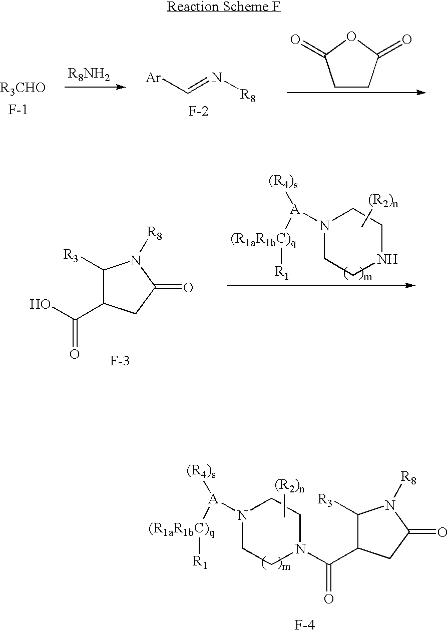

Cyclization of imine F-2, which can be obtained by condensation of an aryl-aldehyde and a primary amine, with succinic anhydride gives the pyrrolidinone F-3, which may be coupled with the substituted piperazine to give the product F-4.

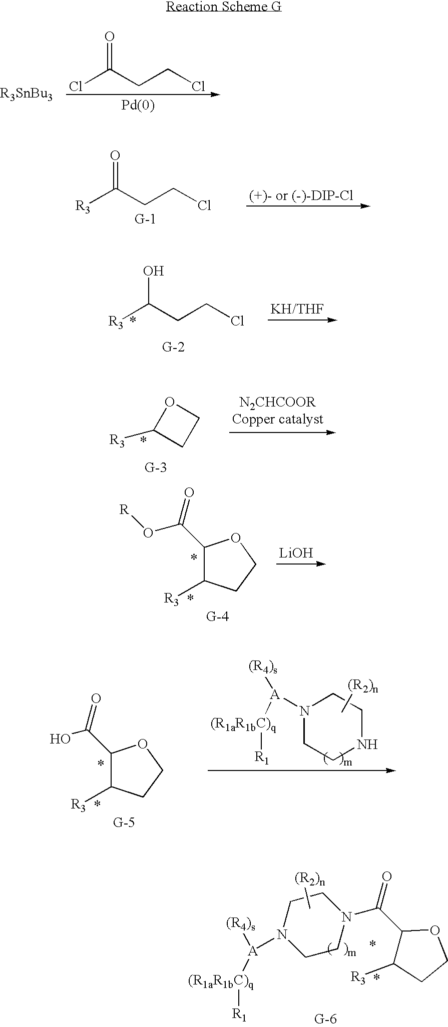

Substituted 3-chloropropinonyl phenone G-1 is reduced to the corresponding alcohol G-2 with the appropriate DIP-chloride (B-chlorodiisopinocampheylborane). This compound is then cyclized to G-3 with KH in a solvent such as THF at 0-100° C. Copper-catalyzed carbene insertion with diazoacetate gives the tetrahydrofuran G-4. Aqueous hydrolysis of G-4 with a base such as lithium hydroxide in an aqueous solvent such as aqueous ethanol at room temperature to reflux gives the corresponding acid G-5. Coupling reaction of G-5 with the substituted piperazine yields the amide G-6 under standard peptide coupling conditions.

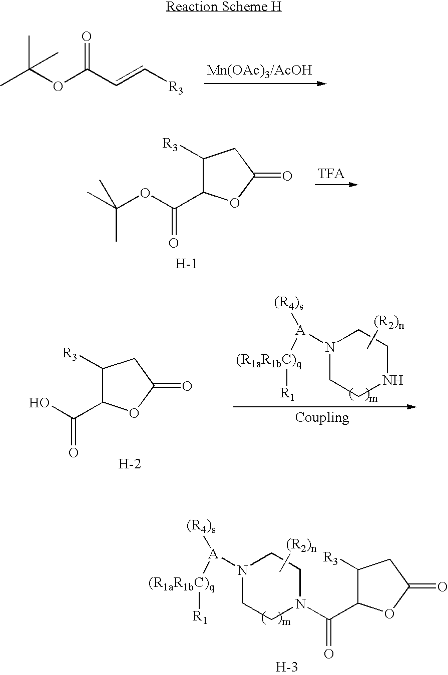

Reaction of a cinnamate with Mn(OAc)₃ in acetic acid gives the cyclic ester H-1, which is hydrolyzed giving the acid H-2. Coupling of H-2 with the substituted piperazine under standard conditions gives the H-3.

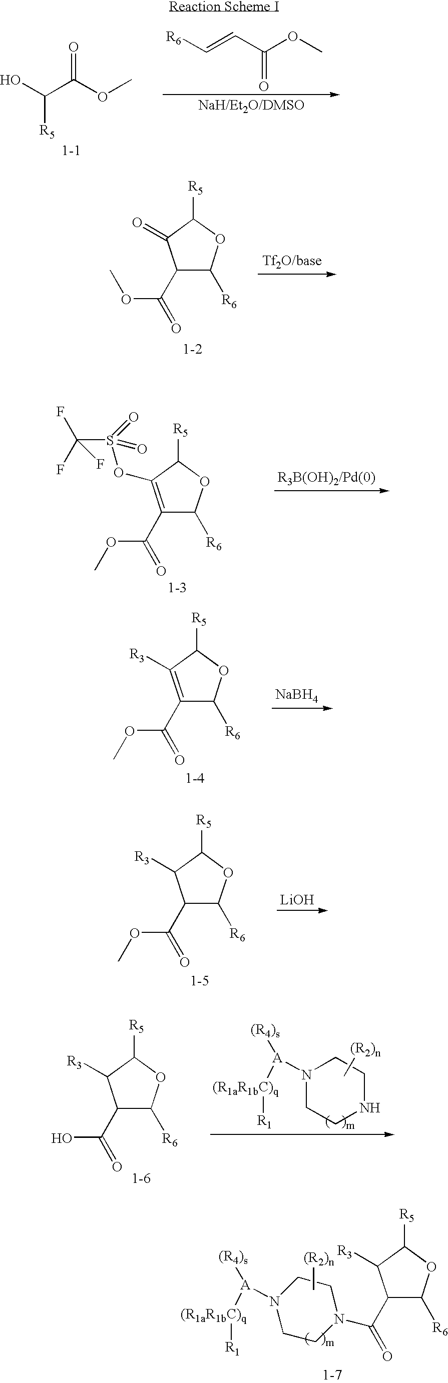

Cyclization of alpha-hydroxy ester I-1 with (un)substituted acrylate in the presence of a base such as sodium hydride in an inert solvent such as ethyl ether, DMSO or combination at a temperature of −30 to 50° C. gives the cyclic ether I-2. Compound I-2 may then be converted to the corresponding triflate I-3 with triflic anhydride in the presence of a base such as triethylamine, and is then subjected to a palladium-catalyzed coupling reaction with an arylboronic acid under Suzuki coupling conditions to give compound I-4. Reduction of I-4 with a reducing agent such as sodium borohydride in a protic solvent such as methanol saturates the double bond to give I-5. Aqueous hydrolysis of I-5 with a base such as lithium hydroxide gives the corresponding acid I-6, which may be coupled to the 4-substituted piperazine to afford the final compound I-7.

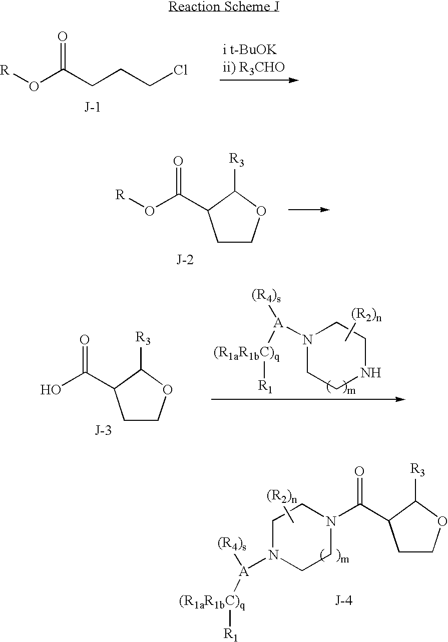

Cyclization of 4-chlorobutyrate J-1 with an aldehyde in the presence of a base such as potassium tert-butoxide in an inert solvent such as ethanol, THF or DMF at a temperature of 0-60° C. gives the tetrahydrofuran J-2. Aqueous hydrolysis of J-2 in a solvent such as ethanol or THF affords the acid J-3, which may be coupled with the 4-substituted piperazine under standard coupling conditions to give the amide J-4.

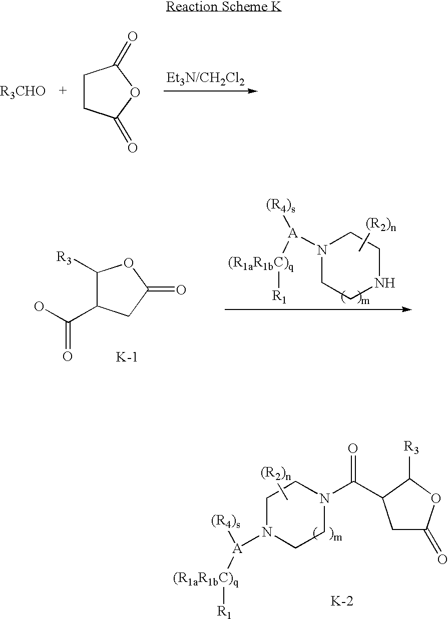

An aryl-aldehyde is cyclized with succinic anhydride in the presence of a base such as triethylamine in an inert solvent such as dichloromethane to give the cyclic ester K-1 which is coupled with the 4-substituted piperazine yielding K-2.

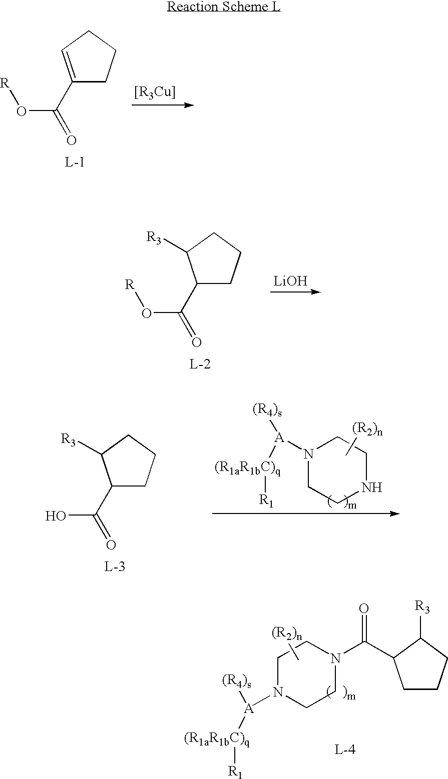

The cyclic unsaturated ester L-1 is subjected to an aryl cuporate addition in an inert solvent such as THF or ether at a temperature of −78 to 60° C. to give the substituted cyclopentane L-2. L-2 is hydrolyzed in an aqueous solvent such as aqueous ethanol with a base such as lithium hydroxide at ambient temperature to give the corresponding acid L-3, which is coupled with the 4-substituted piperazine to give compound L-4.

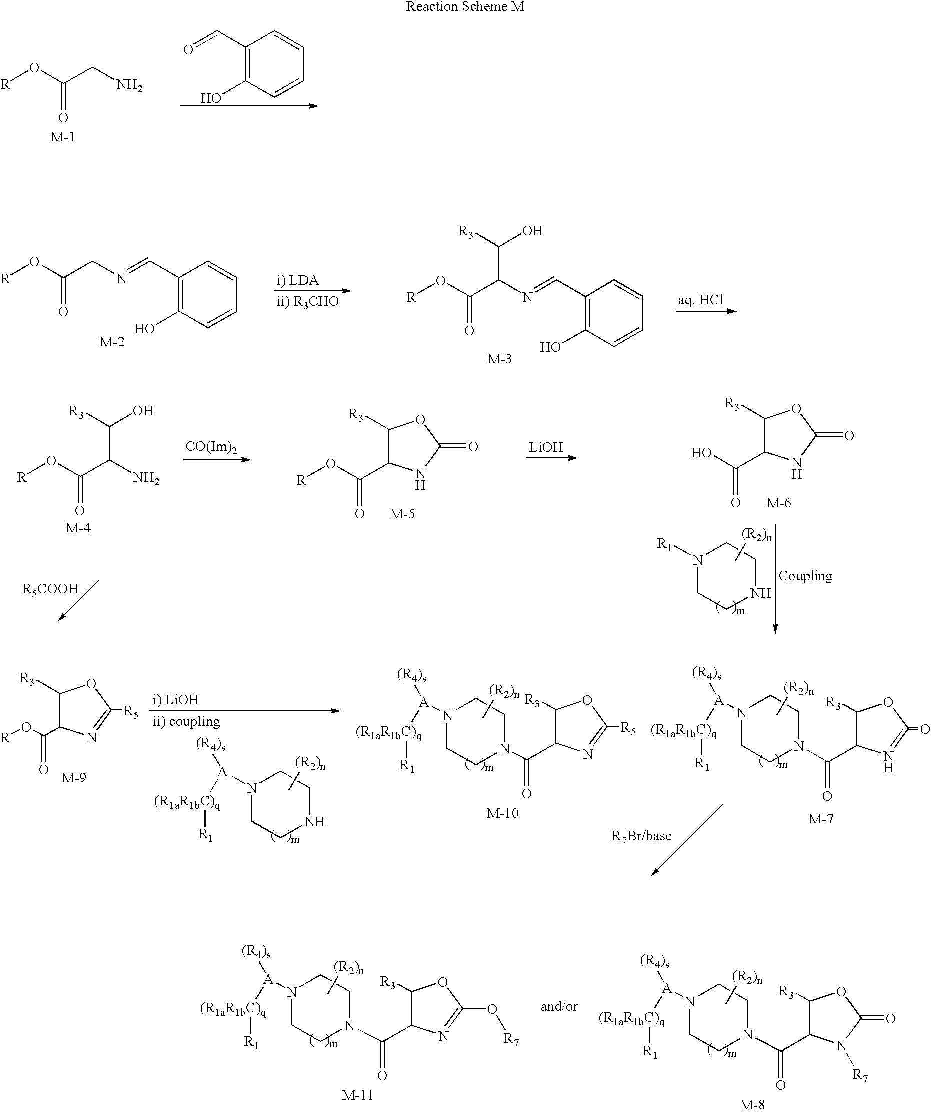

Amino acid ester M-1 is protected by forming an imine M-2 with an aldehyde under dehydration conditions. The imine M-2 is then deprotonated with a strong base such as LDA in an inert solvent such as THF at a low temperature such as between −78 to 0° C., and is quenched with an aryl-aldehyde to afford the alcohol M-3. The imine M-3 is then deprotected under conditions such as aqueous hydrochloric acid to give the amino-alcohol M-4. M-4 is cyclized with a carbonylation reagent such as carbonyl di-imidazole with a base such as triethylamine to give the cyclic carbamate M-5, which is hydrolyzed under basic conditions such as lithium hydroxide in aqueous ethanol to offer the acid M-6. Coupling reaction of M-6 with the 4-substituted piperazine under a standard coupling conditions gives the compound M-7, which may be further modified by alkylation in the presence of a base such as sodium hydride to offer M-8 and/or M-11.

Coupling of M-4 with a carboxylic acid moiety with a coupling reagent such as EDC in an inert solvent such as DMF, followed by cyclization either by heat or acid catalysis gives the oxazoline M-9. Hydrolysis of M-9 with lithium hydroxide, followed by a coupling reaction with the 4-substituted piperazine using a standard coupling conditions such as EDC yields the desired compound M-10.

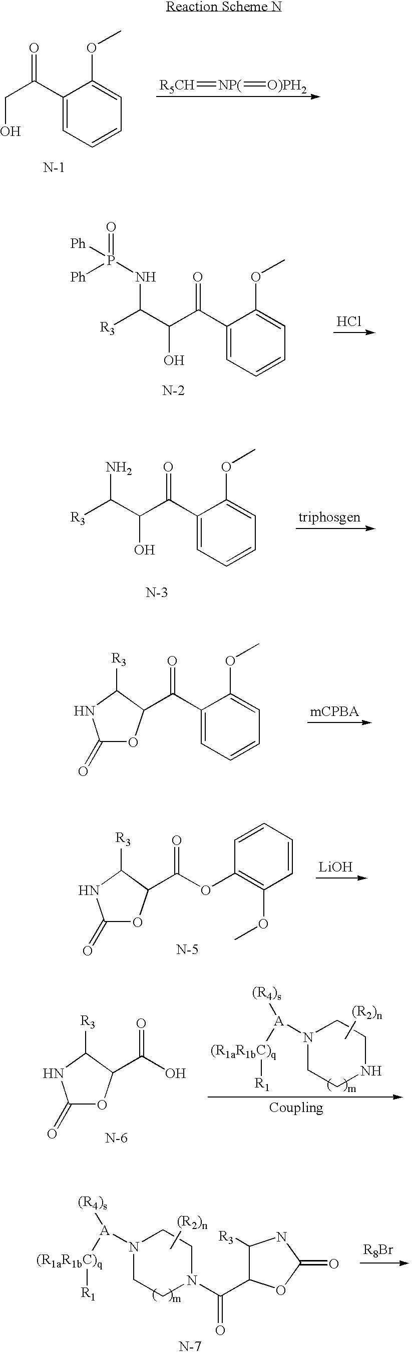

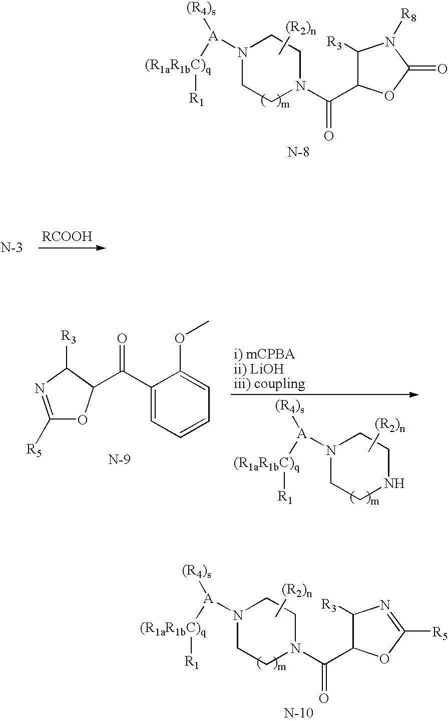

Alpha-hydroxyacetophenone is condensed with the imine moiety N-1 under basic conditions such as LDA to give the alcohol N-2, which is deprotected to give the amino-alcohol N-3. Cyclization of N-3 with a carbonylation reagent such as triphosgene with or without a base affords the cyclic carbamate N-4, which is subjected to a Bayer-Villigar oxidation with a per-cid such as mCPBA in an inert solvent such as chloroform, followed by aqueous hydrolysis under basic conditions to give the acid N-6. N-6 is then coupled with the 4-substituted piperazine to give the product N-7, which may be further modified by alkylation in the presence of a base such as sodium hydride to give N-8. Cyclization of N-3 with a carboxylic acid moiety offers the oxazoline N-9, which, after mCPBA oxidation and aqueous hydrolysis, is coupled with the 4-substituted piperazine to give the product N-10.

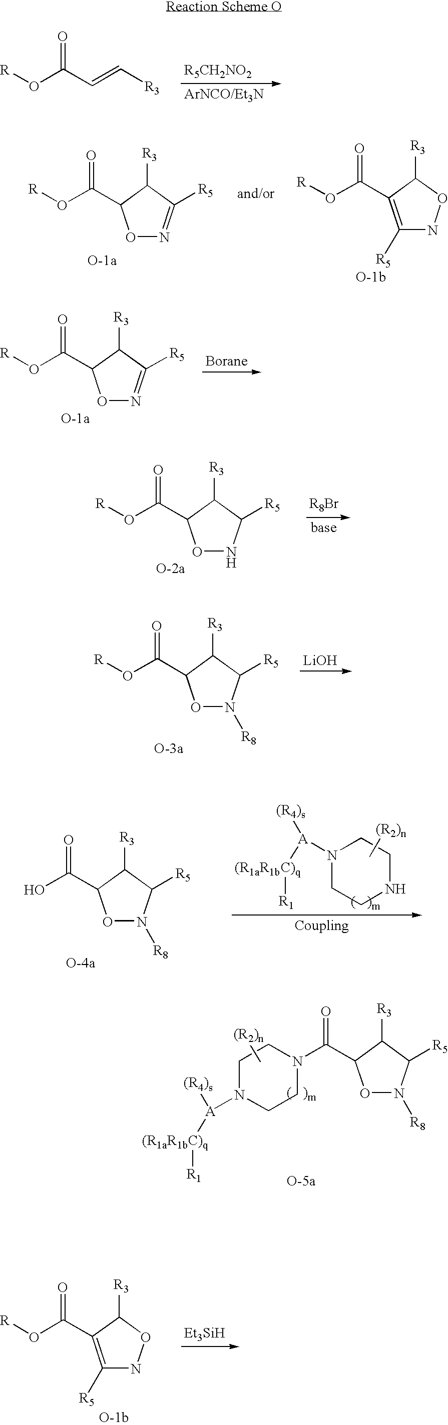

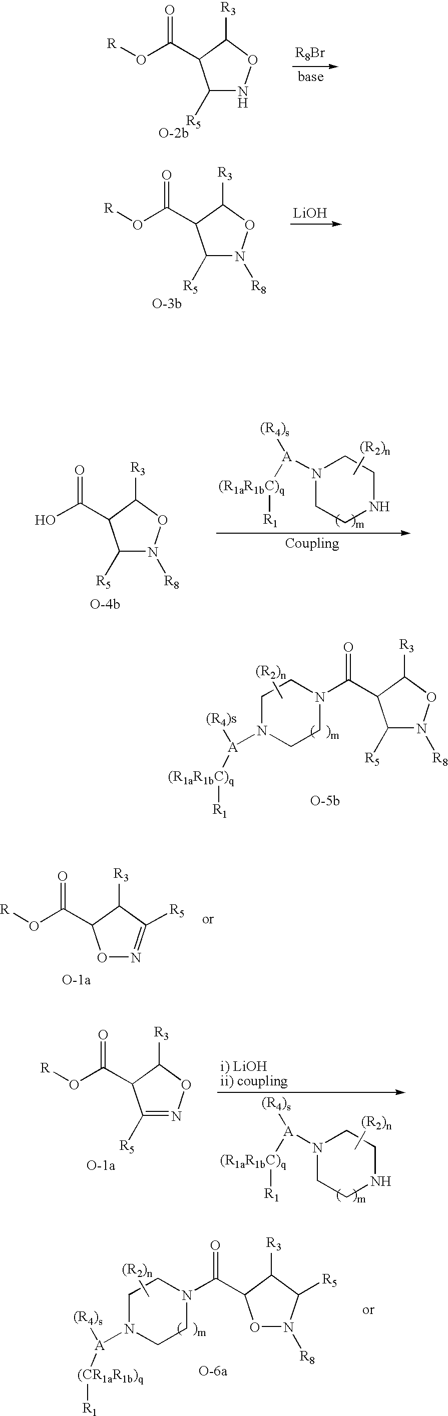

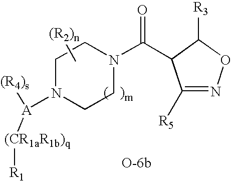

1,3-polar cyclization of nitroalkane with cinnamate promoted by an isocyanate in the presence of a base such as triethylamine gives isooxazoline O-1a or its isomer O-1b. O-1a is reduced with a reducing agent such as borane in an inert solvent such as THF to give O-2a. Alkylation of O-2a with an alkyl halide in the presence of a base such as sodium carbonate gives compound O-3a, which is hydrolyzed in aqueous base such as lithium hydroxide to give the acid O-4a. Coupling of O-4 with the 4-substituted piperazine under standard conditions gives the compound O-5a. Compound O-5b can be synthesized by using a procedure similar to compound O-5a. O-1a (or O-1b) may also be converted to O-6a (or O-6b) by basic hydrolysis, followed by coupling with the 4-substituted piperazine.

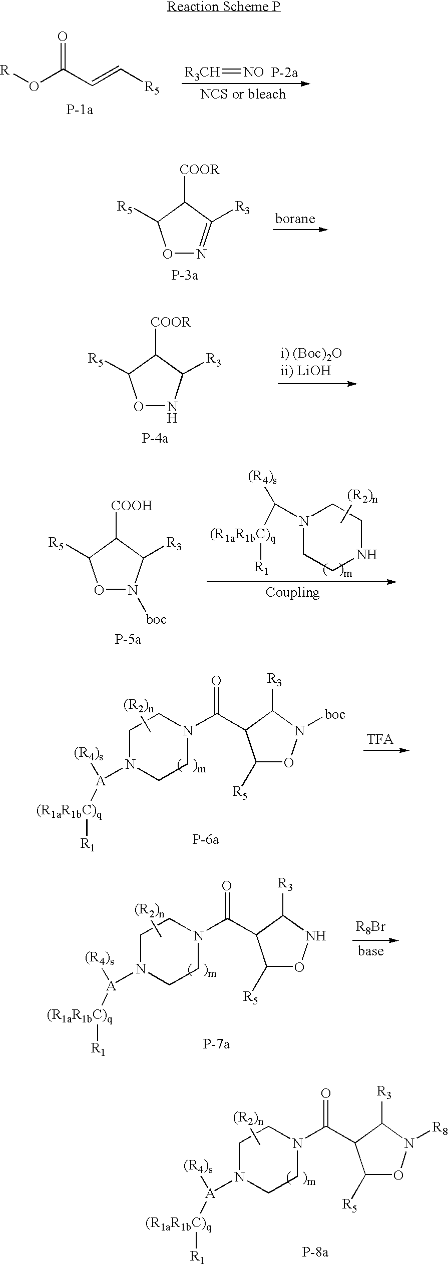

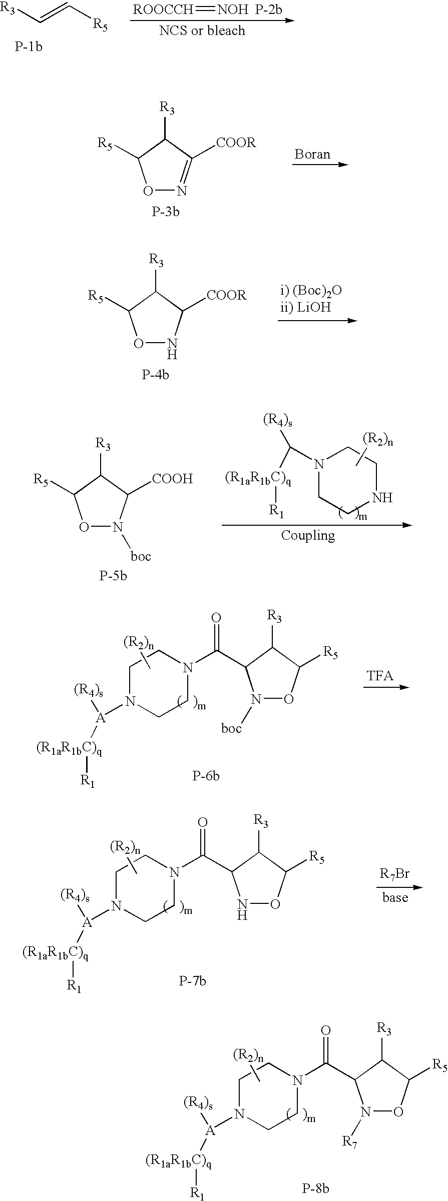

1,3-Dipolar cyclization of olefin P-1a with nitrile N-oxide, after oxidation with N-chlorosuccinimide or bleach, gives the iso-oxazoline P-3a in an inert solvent such as toluene, THFR or dichloroethane at a temperature of O to 100° C. Similarly, P-3b is obtained from P-1b and P-2b. Reduction of the cyclic oxime P-3 with a regent such as borane in an inert solvent such as THF at −30 to 60° C. gives compound P-4. This compound is then protected with a Boc-group and followed by aqueous hydrolysis to give the acid P-5. Coupling reaction of P-5 with the 4-substituted piperazine under standard coupling conditions gives compound P-6. Deprotection of P-6 with TFA or HCl gives the desired compound P-7, which can be further modified by alkylation with an alkyl halide in the presence of a base such as sodium hydride to give compound P-8.

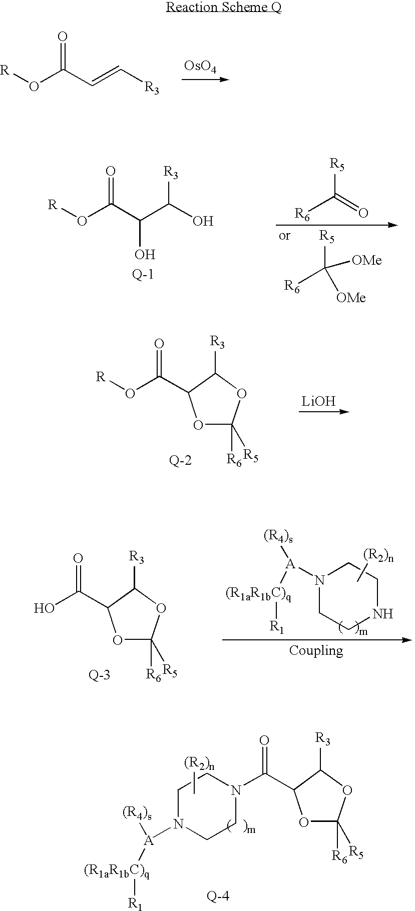

Dihydroxylation of cinnamate with an oxidative reagent such as OsO₄ gives the diol Q-1. Ketal formation of Q-1 is achieved by reaction with an appropriate ketone under dehydration conditions or via a dimethyl ketal catalyzed by acid to give Q-2. Aqueous hydrolysis of Q-2, followed by a coupling reaction with the 4-substituted piperazine gives compound Q-4.

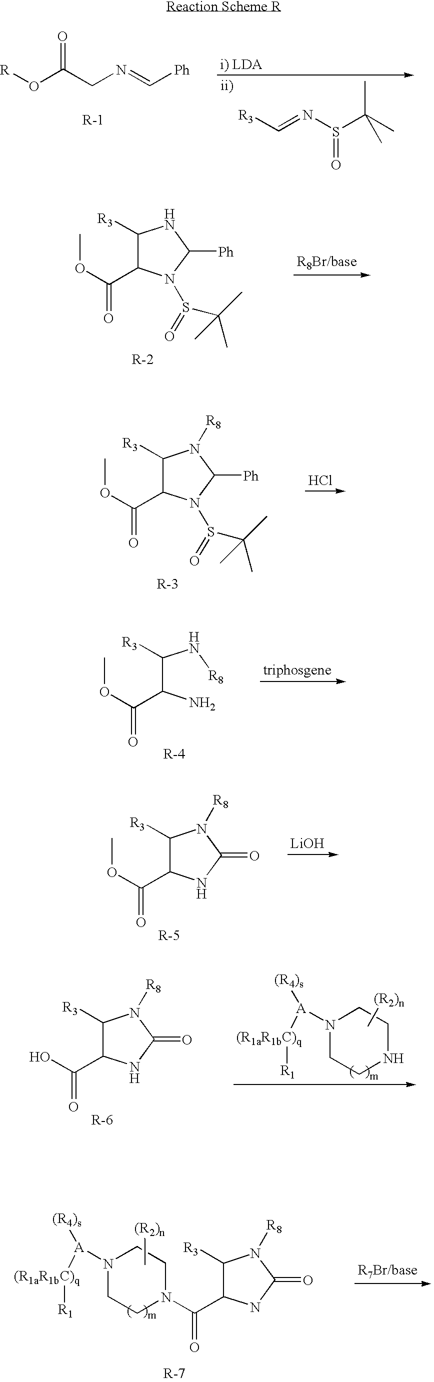

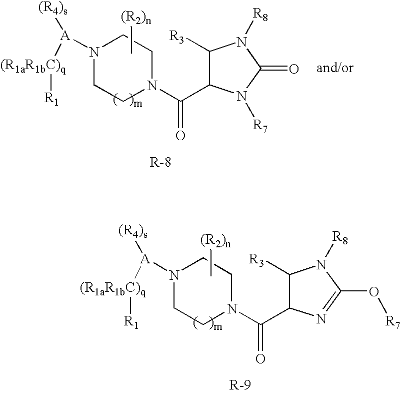

An imine-protected amino acid ester R-1 is deprotected with a base such as LDA in an inert solvent such as THF at a temperature of −78 to 0° C. and then is quenched with the sulfinamide at a temperature of −78° C. to room temperature to give the imidazoline R-2. Alkylation of R-2 with an alkyl halide in the presence of a base such as sodium carbonate gives R-3. Deprotection of R-3 under acidic conditions affords the diamine R-4, which is cyclized with a carbonylation reagent such as triphosgene to give the imidazolinone R-5. R-5 is hydrolyzed under basic conditions to give the acid R-6. Coupling reaction of R-6 with the 4-substituted piperazine yields R-7, which could be further modified to R-8 and/or R-9 by alkylation with an alkyl halide in the presence of a base such as sodium hydride in an inert solvent such as THF.

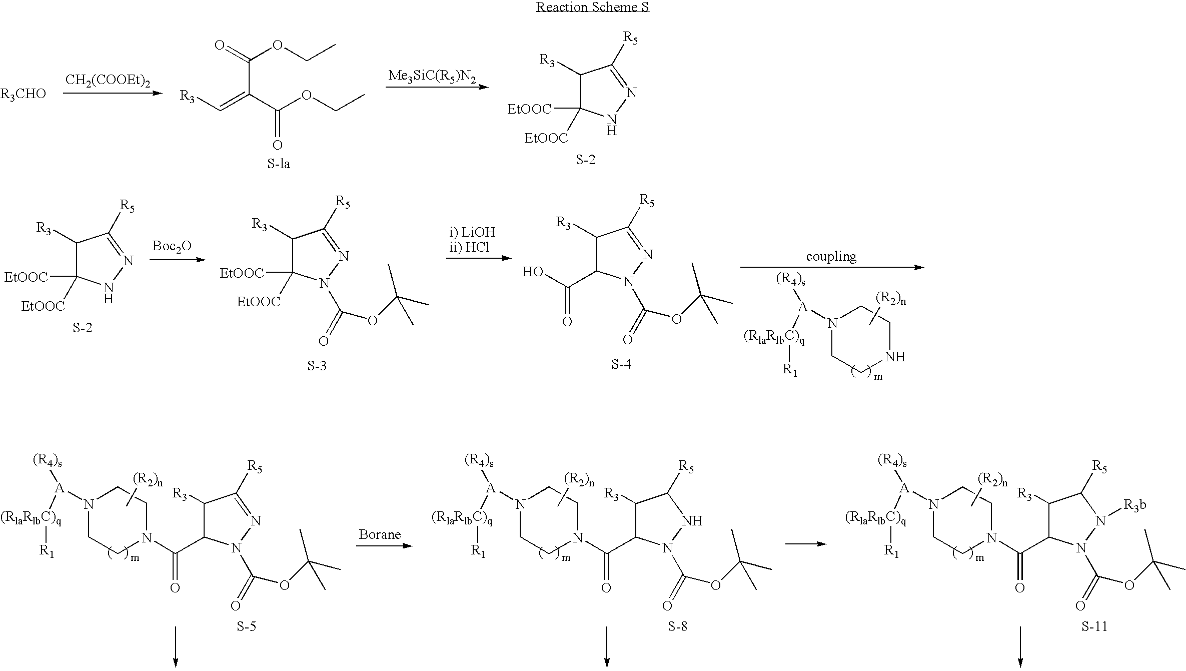

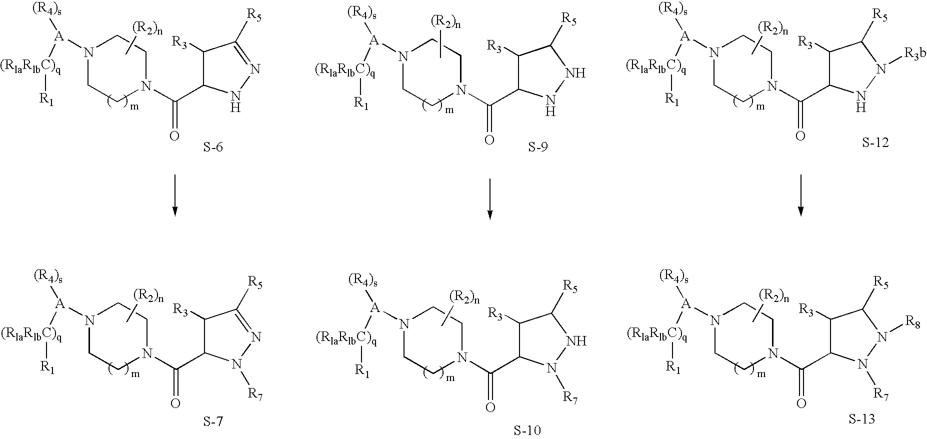

Condensation of malonate with an aryl-aldehyde in the presence of a base such as acetic anhydride at a temperature of 20-100° C. gives the unsaturated ester S-1, which is cyclized with a diazamethyl moiety to give the pyrrolidine S-2. This compound is then protected with a Boc-group to give S-3 followed by hydrolysis with a base such as sodium hydroxide in an aqueous media to give the carboxylic acid S-4. The acid S-3 is coupled with the 4-substituted piperazine under a standard coupling condition to give the pyrrolidine S-5. The Boc group may be- removed using acidic conditions to give S-6, which could be further modified by alkylation with an alkyl halide in the presence of a base such as sodium ethoxide in an inert solvent such as DMF at 0-100° C. to give the S-7.

Reduction of S-5 with a reducing agent such as borane gives the pyrrolidine S-8 in an inert solvent such as THF or toluene at a temperature of 0 to 60° C. Deprotection of the Boc-group was achieved with TFA, and the compound S-9 can be further modified by alkylation with an alkyl halide in the presence of a base such as sodium carbonate in a solvent such as DMF to give S-10.

Alkylation of S-8 with an alkyl halide in the presence of a base such as sodium ethoxide in an inert solvent such as DMF at a temperature of 0 to 100° C. gives compound S-11. Removal of the Boc group affords compound S-12, which can be further modified by alkylation with alkyl halide in the presence of a base such as sodium carbonate to give compound S-13.

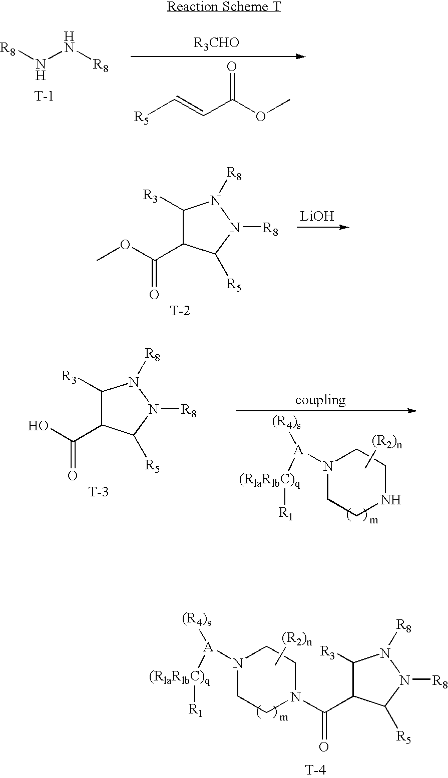

Condensation of hydrazine T-1 with an aldehyde followed by a cyclization with acrylate gives the pyrrolidine T-2. Basic hydrolysis of T-2 gives the corresponding acid T-3 which was coupled with the 4-substituted piperazine under standard conditions to give the final pyrrolidine T-4.

The compounds of the present invention may generally be utilized as the free acid or free base. Alternatively, the compounds of this invention may be used in the form of acid or base addition salts. Acid addition salts of the free amino compounds of the present invention may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of structure (I) is intended to encompass any and all pharmaceutically acceptable salt forms.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

With regard to stereoisomers, the compounds of structure (I) may have chiral centers and may occur as racemates, racemic mixtures and as individual enantiomers or diastereomers. All such isomeric forms are included within the present invention, including mixtures thereof. Compounds of structure (I) may also possess axial chirality which may result in atropisomers. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

The compounds of this invention may be evaluated for their ability to bind to a MC receptor by techniques known in this field. For example, a compound may be evaluated for MC receptor binding by monitoring the displacement of an iodonated peptide ligand, typically [“¹²⁵I]-NDP-α-MSH, from cells expressing individual melanocortin receptor subtypes. To this end, cells expressing the desired melanocortin receptor are seeded in 96-well microtiter Primaria-coated plates at a density of 50,000 cells per well and allowed to adhere overnight with incubation at 37° C. in 5% CO₂. Stock solutions of test compounds are diluted serially in binding buffer (D-MEM, 1 mg/ml BSA) containing [¹²⁵I]-NDP-α-MSH (105 cpm/ml). Cold NDP-α-MSH is included as a control. Cells are incubated with 50 μl of each test compound concentration for 1 hour at room temperature. Cells are gently washed twice with 250 μl of cold binding buffer and then lysed by addition of 50 μl of 0.5 M NaOH for 20 minutes at room temperature. Protein concentration is determined by Bradford assay and lysates are counted by liquid scintillation spectrometry. Each concentration of test compound is assessed in triplicate. IC₅₀ values are determined by data analysis using appropriate software, such as GraphPad Prizm, and data are plotted as counts of radiolabeled NDP-MSH bound (normalized to protein concentration) versus the log concentration of test compound.

In addition, functional assays of receptor activation have been defined for the MC receptors based on their coupling to Gs proteins. In response to POMC peptides, the MC receptors couple to G_{S} and activate adenylyl cyclase resulting in an increase in cAMP production. Melanocortin receptor activity can be measured in HEK293 cells expressing individual melanocortin receptors by direct measurement of cAMP levels or by a reporter gene whose activation is dependent on intracellular cAMP levels. For example, HEK293 cells expressing the desired MC receptor are seeded into 96-well microtiter Primaria-coated plates at a density of 50,000 cells per well and allowed to adhere overnight with incubation at 37° C. in 5% CO₂ Test compounds are diluted in assay buffer composed of D-MEM medium and 0.1 mM isobutylmethylxanthine and assessed for agonist and/or antagonist activity over a range of concentrations along with a control agonist α-MSH. At the time of assay, medium is removed from each well and replaced with test compounds or α-MSH for 30 minutes at 37° C. Cells are harvested by addition of an equal volume of 100% cold ethanol and scraped from the well surface. Cell lysates are centrifuged at 8000×g and the supernatant is recovered and dried under vacuum. The supernatants are evaluated for cAMP using an enzyme-linked immunoassay such as Biotrak, Amersham. EC₅₀ values are determined by data analysis using appropriate software such as GraphPad Prizm, and data are plotted as cAMP produced versus log concentration of compound.

As mentioned above, compounds of this invention may function as ligands to one or more MC receptors, and therefore may be useful in the treatment of a variety of conditions or diseases associated therewith. In this manner, the ligands may function by altering or regulating the activity of an MC receptor, thereby providing a treatment for a condition or disease associated with that receptor. Consequently, compounds of this invention may have utility over a broad range of therapeutic applications, and may be used to treat disorders or illnesses, including (but not limited to) eating disorders, cachexia, obesity, diabetes, metabolic disorders, inflammation, pain, skin disorders, skin and hair coloration, male and female sexual dysfunction, erectile dysfunction, dry eye, acne and/or Cushing's disease.

Compounds of the present invention may also be used in combination therapy with agents that modify sexual arousal, penile erections, or libido such as sildenafil, yohimbine, apomorphine or other agents. Combination therapy with agents that modify food intake, appetite or metabolism are also included within the scope of this invention. Such agents include, but are not limited to, other MC receptor ligands, ligands of the leptin, NPY, melanin concentrating hormone, serotonin or B₃ adrenergic receptors.

In another embodiment, the present invention includes pharmaceutical compositions containing one or more compounds of this invention. For the purposes of administration, the compounds of the present invention may be formulated as pharmaceutical compositions. Pharmaceutical compositions of the present invention comprise pharmaceutically effective amount of a compound of structure (I) and a pharmaceutically acceptable carrier and/or diluent. Thus, the compound is present in the composition in an amount which is effective to treat a particular disorder of interest, and preferably with acceptable toxicity to the patient. Typically, the pharmaceutical composition may include a compound of this invention in an amount ranging from 0.1 mg to 250 mg per dosage depending upon the route of administration, and more typically from 1 mg to 60 mg. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets that contain, in addition to a compound of this invention, dispersing and surface active agents, binders, and lubricants. One skilled in this art may further formulate the compound in an appropriate manner, and in accordance with accepted practices, such as those disclosed in Remington's Pharmaceutical Sciences, Gennaro, Ed., Mack Publishing Co., Easton, Pa. 1990.

In another embodiment, the present invention provides a method for treating a condition associated with the activity of an MC receptor. Such methods include administration of a compound of the present invention to a warm-blooded animal in an amount sufficient to treat the condition. In this context, “treat” includes prophylactic administration. Such methods include systemic administration of compound of this invention, preferably in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions that may contain buffers, antioxidants, bacteriostats, and other additives commonly employed in such solutions.

The following examples are provided for purposes of illustration, not limitation.

### EXAMPLES

Aqueous Work Up

The reaction mixture was concentrated under a stream of nitrogen, taken up in dichloromethane, washed with aqueous sodium bicarbonate, and again concentrated. Final compounds were dissolved in methanol and filtered prior to preparative HPLC purification.

Analytical Procedures

A—Analytical HPLC-MS (LC-MS)

HP 1100 series: equipped with an auto-sampler, an UV detector (220 nM and 254 nM), a MS detector (electrospray);

HPLC column: YMC ODS AQ, S-5, 5μ, 2.0×50 mm cartridge;

HPLC gradients: 1.5 mL/minute, from 10% acetonitrile in water to 90% acetonitrile in water in 2.5 minutes, maintaining 90% for 1 minute.

B—Prep. HPLC-MS

Gilson HPLC-MS equipped with Gilson 215 auto-sampler/fraction collector, an UV detector and a ThermoFinnigan AQA Single QUAD Mass detector (electrospray);

HPLC column: BHK ODS-O/B, 5μ, 30×75 mm

HPLC gradients: 35 mL/minute, 10% acetonitrile in water to 1 00% acetonitrile in 7 minutes, maintaining 100% acetonitrile for 3 minutes.

C—Analytical HPLC-MS (LC-MS)

HP 1100 series: equipped with an auto-sampler, an UV detector (220 nM and 254 nM), a MS detector (electrospray);

HPLC column: YMC ODS AQ, S-5, 5μ, 2.0×50 mm cartridge;

HPLC gradient: 1.5 mL/minute, from 10% acetonitrile in water to 90% acetonitrile in water in 2.5 minutes, maintaining 90% for 1 minute. Both acetonitrile and water have 0.025% TFA.

D—Analytical HPLC-MS (LC-MS)

HP 1100 series: equipped with an auto-sampler, an UV detector (220 nM and 254 nM), a MS detector (electrospray);

HPLC column: Phenomenex Synergi-Max RP, 2.0×50 mm column;

HPLC gradient: 1.0 mL/minute, from 5% acetonitrile in water to 95% acetonitrile in water in 13.5 minutes, maintaining 95% for 2 minute. Both acetonitrile and water have 0.025% TFA.

E—Analytical HPLC-MS (LC-MS)

HP 1100 series: equipped with an auto-sampler, an UV detector (220 nM and 254 nM), a MS detector (electrospray);

HPLC column: XTerra MS, C₁₈, 5μ, 3.0×250 mm cartridge;

HPLC gradient: 1.0 mL/minute, from 5% acetonitrile in water to 90% acetonitrile in water in 47.50 minutes, maintaining 99% for 8.04 minutes. Both acetonitrile and water have 0.025% TFA.

F—Analytical HPLC-MS (LC/MS)

Gilson 333/334 series: equipped with a Gilson 215 Liquid-Handler, a Gilson UV/VIS-156 UV detector (220 nM and 254 nM) and Finnigan AQA Mass Spec (ElectroSpray);

HPLC column: BHK Alpha, C-18, 5μ, 120A, 4.6×150 mm cartridge (PN: OB511546);

HPLC gradient: 3.6 mL/minute, maintaining 10% acetonitrile in water for 1 minute. Increasing from 10% acetonitrile in water to 90% acetonitrile in water over 12 minutes. Then increasing to 99% in 0.1 minutes and maintaining for 1.5 minutes. Both acetonitrile and water have 0.05% TFA.

G—Analytical HPLC-MS (SFC-MS)

HP 1100 series: equipped with an auto-sampler, an UV detector (220 nM and 254 nM), a MS detector (electrospray) and FCM 1200 CO₂ pump module;

HPLC column: Berger Pyridine, PYR 60A, 6μ, 4.6×150 mm column;

HPLC gradient: 4.0 mL/minute, 120 bar; from 10% methanol in supercritical CO₂ to 60% methanol in supercritical CO₂ in 1.67 minutes, maintaining 60% for 1 minute. Methanol has 1.5% water. Backpressure regulated at 140 bar.

H—Analytical HPLC (HPLC)

Shimadzu SIL-10A series: equipped with an auto-sampler and UV detector (220 nM and 254 nM);

HPLC column: ZORBAX SB-C18, 5μ, 4.6×250 mm cartridge (PN: 880975-902);

HPLC gradient: 2.0 mL/minute, maintaining 5% acetonitrile in water for 4 minutes then to 10% acetonitrile in 0.1 min and 10% acetonitrile in water to 95% acetonitrile in water in 46 minutes, then increasing to 99% in 0.1 minutes and maintaining for 10.8 minutes. Both acetonitrile and water have 0.025% TFA.

I—Analytical HPLC (HPLC)

HP 1100 series: equipped with an auto-sampler and UV detector (220 nM and 254 nM);

HPLC column: Waters Symetry, C-8, 5μ, 4.6×150 mm cartridge (PN: WAT045995);

HPLC gradient: 2.8 mL/minute, maintaining 5% acetonitrile in water for 1 minute. Increasing to 10% acetonitrile in water in 0.1 minutes. Then increasing to 90% acetonitrile in water in 15 minutes. Then increasing to 99% in 0.1 minutes and maintaining for 2.4 minutes. Both acetonitrile and water have 0.05% TFA.

### Example 1

### 4-[4-(trifluoromethyl)-2-(1S-amino-3-methylbutyl)phenyl]-1-[1-isopropyl-3-(4-chlorophenyl)pyrrolidinecarbonyl]piperazine

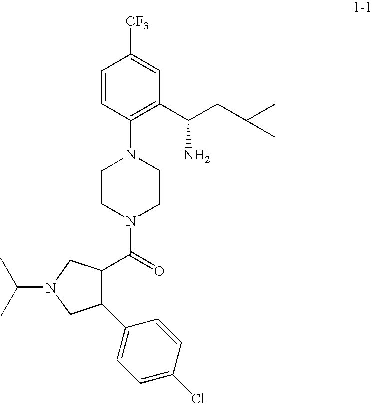

### Step 1A. 2-[4′-(tert-Butoxycarbonyl)-1-piperazinyl]-5-trifluoromethyl-benzaldehyde 1a

To a solution of 2-fluoro-5-trifluoromethylbenzaldehyde (10.0 mL, 68.7 mmol) and 1-BOC-piperazine (15.4 g, 82.4 mmol) in 140 mL of DMF was added K₂CO₃ (47.4 g, 344 mmol). The reaction mixture was heated and stirred at 120° C. for 10 hours. The reaction mixture was cooled to room temperature and diluted with 200 mL of EtOAc. The mixture was filtered, and the filter was washed well with EtOAc (3×50 mL). The filtrate was washed with 5% aqueous HCl (100 mL) and the aqueous layer was extracted with EtOAc (3×25 mL). The combined organic layers were washed with H₂O (2×40 mL) and brine (50 mL). After drying (MgSO₄), and concentration in vacuo, the residue was triturated with hexanes (3×20 mL) to give a brown oil. The brown oil slowly solidified to give the compound 1a as a yellow solid (22.3 g, 92%).

### Step 1B. 2-[4-(tert-Butoxycarbonyl)-1-piperazinyl]-5-trifluoromethyl-benzylidene}-t-butanesulfinamide 1b

To a THF (41 mL) solution of aldehyde 1a (3.29 g, 9.18 mmol) at room temperature was added Ti(OEt)₄ (tech. Grade, Ti˜20%, contains excess ethanol, 9 m]L, 36.7 mmol), and (S)-(−)-2-methyl-2-propanesulfinamide (1.26 g, 10.1 mmol) and the mixture was stirred overnight. The reaction mixture was poured into a saturated aqueous NaCl solution (30 mL) at room temperature with vigorous stirring and the resulting suspension was filtered through Celite®, and the filter cake was washed with EtOAc (500 mL). After phase separation, the aqueous layer was extracted with EtOAc (30 mL) and the combined organic layers were dried over Na₂SO₄ and evaporated to provide a residue which was purified by 5˜10% EtOAc/Hexanes triturating to give 4.20 g of 1b as a light yellow powder (99%).

### Step 1C. 2-[4-(tert-Butoxycarbonyl)-1-piperazinyl]-1-[1S-(S-t-butanesulfinamido)-3-methylbutyl]-5-trifluoromethylbenzene 1c

To a THF (25 mL) solution of sulfinyl aldimine 1b (4.20 g, 9.10 mmol) was added trimethylaluminum (2.0 M in toluene or heptane or hexane, 9.10 mL, 18.2 mmol) at −40° C. and the mixture was stirred for 20 minutes. The mixture was cooled to −78° C. and i-BuLi (1.6 M in heptane from Fluka, 11.4 mL, 18.2 mmol) was added to this mixture by syringe pump at 1.2 m”L/min. After i-BuLi addition, the reaction mixture was stirred for 30 minutes at −78° C., quenched with a 5% aqueous HCl (25 mL) at −78° C., warmed to 10° C. and extracted with EtOAc (3×50 mL). The combined organic layers were washed with brine (30 mL) and dried over Na₂SO₄ then evaporated to provide a crude oil which was purified by 10˜25% EtOAc/Hexanes chromatography to give 4.00 g of compound 1c as a white foam (85% yield).

Starting with the appropriate fluoroaldehydes and alkyllithiums and following the procedures outlined in Steps 1A to 1C, the following compounds were also synthesized:
2-[4-(tert-Butoxycarbonyl)-1-piperazinyl]-1-[1S-(S-t-butanesulfinamido)-3-methylbutyl]-5-chlorobenzene 1c.a
2-[4-(tert-Butoxycarbonyl)-1-piperazinyl]-1-[1S-(S-t-butanesulfinamido)-3-methylbutyl]-3-fluorobenzene 1c.b
2-[4-(tert-Butoxycarbonyl)-1-piperazinyl]-1-[1S-(S-t-butanesulfinamido)-3-methylbutyl]-5-methylbenzene 1c.c
2-[4-(tert-Butoxycarbonyl)-1-piperazinyl]-1-[1S-(S-t-butanesulfinamido)-2-methylpropyl]-3-fluorobenzene 1c.d
2-[4-(tert-Butoxycarbonyl)-1-piperazinyl]-1-[1S-(S-t-butanesulfinamido)-2-methylpropyl]-5-methylbenzene 1c.e.

### Step 1D. 2-{4-[1-(tert-Butoxycarbonyl)-3-(4-chlorophenyl)-1-pyrrolidinecarbonyl]-1-piperazinyl}-1-[1S-(S-t-butanesulfinamido)-3-methylbutyl-5-trifluoromethylbenzene 1d

To a dichloromethane (18 mL) solution of 2-[4-(tert-butoxycarbonyl)-1-piperazinyl]-1-[1S-(S-t-butanesulfinamido)-3-methylbutyl]-5-trifluoromethylbenzene 1c (1.02 g, 2.17 mmol) was added TFA (4.5 mL) at 23° C. and the mixture was stirred for 45 minutes. The reaction mixture was treated with saturated aqueous NaHCO₃ solution (100 mL) and was extracted with EtOAc (2×100 mL). The organic layer was dried over Na₂SO₄ and then was evaporated to provide the piperazine 1c.1 as a white foam which was dissolved in DMF/dichloromethane (1:3, 12 mL). To this solution was added NaHCO₃ (0.365 g, 4.34 mmol), 1-[(tert-butyl)oxycarbonyl]-4-(4-chlorophenyl)pyrrolidine-3-carboxylic acid (0.851 g, 2.61 mmol), HOBt (0.352 g, 2.61 mmol), EDCI (0.500 g, 2.61 mmol) sequentially. The reaction mixture was stirred overnight at room temperature. The mixture was diluted with EtOAc (60 mL), washed with 5% aqueous HCl (15 mL), saturated aqueous NaHCO₃ (15 mL), and brine (15 mL), and then was dried (Na₂SO₄). The solution was concentrated in vacuo to provide a residue which was purified by flash column chromatography (30˜60% EtOAc in Hexanes) to provide the compound 1d. (1.2 g, 87%). MS: 524 (M+H-Boc)

### Step 1E. 2-{4-[3-(4-Chlorophenyl)-1-pyrrolidinecarbonyl]-1-piperazinyl}-1-[1S-(S-t-butanesulfinamido)-3-methylbutyl]-5-trifluoromethylbenzene 1e

To a dichloromethane (4 mL) solution of 2-{4-[1-(tert-Butoxycarbonyl)-3-(4-chlorophenyl)-1-pyrrolidinecarbonyl]-1-piperazinyl}-1-[1S-(S-t-butanesulfinamido)-3-methylbutyl]-5-trifluoromethylbenzene 1d (320 mg, 0.494 mmol) was added TFA (1 mL) at 23° C. and the mixture was stirred for 60 minutes. The reaction mixture was treated with saturated aqueous NaHCO₃ solution (30 mL) and extracted with EtOAc (2×30 mL). The organic layer was dried over Na₂SO₄ and evaporated to provide the free amine 1e as a white foam. MS: 524 (MH⁺)

### Step 1F. 2-{4-[1-Isopropyl-3-(4-chlorophenyl)-1-pyrrolidinecarbonyl]-1-piperazinyl}-1-[1S-amino-3-methylbutyl]-5-trifluoromethylbenzene 1-1

2-{4-[3-(4-Chlorophenyl)-1-pyrrolidinecarbonyl]-1-piperazinyl}-1-[1S-(S-t-butanesulfinamido)-3-methylbutyl]-5-trifluoromethylbenzene 1e (62.7 mg, 0.1 mmol) was dissolved in 1,2-dichloroethane (0.5 mL) along with acetone (7.3 μL, 0.1 mmol) and acetic acid (5.7 μL, 0.1 mmol). The mixture was stirred at room temperature for 1 hour then NaBH(OAc)₃ (29.7 mg, 0.14 mmol) was added. The reaction stirred at room temperature for an additional 8 hours then was quenched with saturated NaHCO₃ solution (2 mL). The organic layer was separated and concentrated under a stream of nitrogen. The residue was dissolved in 2 mL of MeOH and 0.5 mL of 2N HCl in ether was added. The reaction was stirred at room temperature for 1 hour then solvent was removed by evaporating under a stream of nitrogen and the crude product was purified by preparative HPLC. The compound 1-1 was recovered as the TFA salt in 17.3% overall yield from the benzaldehyde. MS: calc. for C₃₀H₄₀ClF₃N₄O: 564.28; Found: 565 (MH⁺); retention time: 7.45 minutes; Method info: APCI positive ion scan 100-1000 Frag V=80; 95% 0.05%TFA/H₂O to 95% ACN/0.05% TFA over 13 min, 15.5 min run, ODS-AQ column.

By the above procedures, the compounds of the following Table 1 were prepared.

TABLE 1

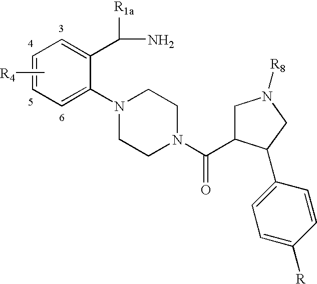

| Cpd | R₄ | R₁ₐ | R₈ | R | MW | (MH⁺) |
|---|---|---|---|---|---|---|
| 1-1 | 4-CF₃ | iBu | iPr | Cl | 565.1 | 565 |
| 1-2 | 4-CF₃ | iBu | Bn | Cl | 613.2 | 613 |
| 1-3 | 4-CF₃ | iBu | NH₂CH₂CH₂— | Cl | 566.1 | 566 |
| 1-4 | 4-CF₃ | iBu | H | Cl | 523.0 | 523 |
| 1-5 | 4-CF₄ | iBu | H | MeO- | 518.6 | |
| 1-6 | 4-CF₃ | iBu | iPr | MeO- | 560.7 | 561 |
| 1-7 | 4-CF₃ | iBu | 2-Pn | MeO- | 588.8 | 589 |
| 1-8 | 4-CF₃ | iBu | iBu | MeO- | 574.7 | 575 |
| 1-9 | 6-F | iPr | iPr | Cl | 501.1 | 501 |
| 1-10 | 6-F | iPr | Bn | Cl | 549.1 | 549 |
| 1-11 | 6-F | iPr | iPr | Cl | 497.1 | 497 |
| 1-12 | 4-Me | iPr | Bn | Cl | 545.2 | 545 |
| 1-13 | H | H | iPr | Cl | 441.0 | 441 |
| 1-14 | 6-F | iPr | H | Cl | 459.0 | 459 |
| 1-15 | 6-F | iPr | cyclobutyl | Cl | 513.1 | 513 |
| 1-16 | 6-F | iPr | cyclopentyl | Cl | 527.1 | 527 |
| 1-17 | 6-F | iPr | Me | Cl | 473.0 | 473 |
| 1-18 | 6-F | iPr | Et | Cl | 487.1 | 487 |
| 1-19 | 6-F | iPr | Pr | Cl | 501.1 | 501 |
| 1-20 | 6-F | iPr | iBu | Cl | 515.1 | 515 |
| 1-21 | 6-F | iPr | HOCH₂CH₂— | Cl | 503.1 | 503 |
| 1-22 | 6-F | iPr | CF₃CH₂CH₂— | Cl | 555.1 | 555 |
| 1-23 | 4-Cl | iBu | cyclopentyl | Cl | 557.6 | 557 |
| 1-24 | 4-Cl | iBu | cyclohexyl | Cl | 571.6 | 571 |
| 1-25 | 4-Me | iPr | H | Cl | 455.0 | |
| 1-26 | 4-Me | iPr | 2-methyl-3- | Cl | 539.2 | 539 |
| | | | tetrahydro-furanyl | | | |
| 1-27 | 4-Me | iPr | MeOCH₂CH(Me)— | Cl | 527.1 | 527 |
| 1-28 | 4-Me | iPr | (MeOCH₂)₂CH— | Cl | 557.2 | 557 |
| 1-29 | 4-Me | iPr | 2-methoxy- | Cl | 567.2 | 567 |
| | | | cyclohexyl | | | |
| 1-30 | 4-Me | iPr | 2,2,5,5- | Cl | 581.2 | 581 |
| | | | tetramethyl- | | | |
| | | | tetrahydro-3- | | | |
| | | | furanyl | | | |
| 1-31 | 4-Me | iPr | cyclohexyl | Cl | 537.2 | 537 |
| 1-32 | 4-Me | iPr | 1-ethyl-4- | Cl | 566.2 | 566 |
| | | | piperidinyl | | | |
| 1-33 | 4-Me | iPr | 1-isopropyl-4- | Cl | 580.3 | 580 |
| | | | piperidinyl | | | |
| 1-34 | 4-Me | iPr | 1-Boc-4- | Cl | 638.3 | 638 |
| | | | piperidinyl | | | |
| 1-35 | 4-Me | iPr | 1-isobutyl-4- | Cl | 594.3 | 594 |
| | | | piperidinyl | | | |
| 1-36 | 4-Me | iPr | 1-acetyl-4- | | | |
| | | | piperidinyl | Cl | 580.2 | 580 |

### Example 2

### 2-{4-[1-acetyl-3-(4-chlorophenyl)-1-pyrrolidinecarbonyl]-1-piperazinyl}-1-[1S-amino-3-methylbutyl]-5-trifluoromethylbenzene

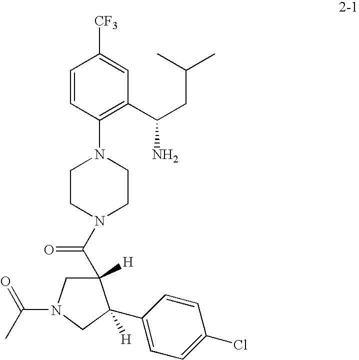

### Step 2A. 2-{4-[1-Acetyl-3-(4-chlorophenyl)-1-pyrrolidinecarbonyl]-1-piperazinyl}-1-[1S-amino-3-methylbutyl]-5-trifluoromethylbenzene 2-1

2-{4-[3-(4-Chlorophenyl)-1-pyrrolidinecarbonyl]-1-piperazinyl}-1-[1S-(S-t-butanesulfinamido)-3-methylbutyl]-5-trifluoromethylbenzene (1e, 62.7 mg, 0.1 mmol) was dissolved in THF (0.5 mL) along with triethylamine (13.9 uL, 0.1 mmol). To the reaction mixture, acetyl chloride (7.1 mg, 0.1 mmol) was added and the reaction stirred at room temperature for 8 hours. Solvent was then removed by evaporating under a stream of nitrogen. The residue was dissolved in 1 mL of dichloromethane and was washed with saturated NaHCO₃ solution (2 mL). The organic layer was evaporated to dryness and diluted with 2 mL of MeOH. To the reaction mixture, 2N HCl (0.5 mL) was added and the reaction was stirred at room temperature for 1 hour. Solvent was removed by evaporating under a stream of nitrogen and the crude product was purified by preparative HPLC. The compound 2-1 was recovered as the TFA salt in 29.8% overall yield from the 2-fluoro-5-trifluoromethylbenzaldehyde of Step 1A. MS: calc. for C₂₉H₃₆ClF₃N₄O₂: 564.25; Found: 565 (MH⁺); retention time: 9.275 minutes; Method info: APCI positive ion scan 100-1000 Frag V=80; 95% 0.05% TFA/H₂O to 95% ACN/0.05%TFA over 13 min, 15.5 min run, ODS-AQ column.

By the above procedures, the compounds of the following Table 2 were prepared.

TABLE 2

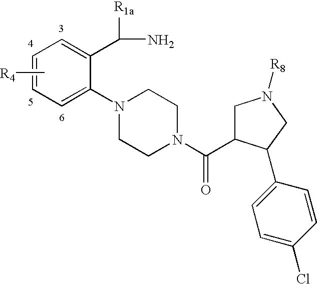

| Cpd | R₄ | R₁ₐ | R₈ | MW | (MH⁺) |
|---|---|---|---|---|---|
| 2-1 | 4-CF₃ | iBu | tBuOC(O)— | 623.2 | 623 |
| 2-2 | 4-CF₃ | iBu | MeC(O)— | 565.1 | 565 |
| 2-3 | 4-CF₃ | iBu | PhC(O)— | 627.1 | 627 |
| 2-4 | 6-F | iPr | MeC(O)— | 501.0 | 501 |
| 2-5 | 6-F | iPr | tBuC(O)— | 543.1 | 543 |
| 2-6 | 6-F | iPr | PrC(O)— | 529.1 | 529 |
| 2-7 | 6-F | iPr | PhC(O)— | 563.1 | 563 |
| 2-8 | 6-F | iPr | iPrC(O)— | 529.1 | 529 |
| 2-9 | 6-F | iPr | CyclohexylC(O)— | 569.2 | 569 |
| 2-10 | 6-F | iPr | 3-pentylC(O)— | 557.2 | 557 |
| 2-11 | 4-Cl | iBu | MeC(O)— | 531.5 | 531 |
| 2-12 | 4-Cl | iBu | EtC(O)— | 545.6 | 545 |
| 2-13 | 4-Cl | iBu | PrC(O)— | 559.6 | 559 |
| 2-14 | 4-Cl | iBu | Cyclobutyl-C(O)— | 571.6 | 571 |
| 2-15 | 4-Me | iPr | MeC(O)— | 497.1 | 497 |
| 2-16 | 4-Me | iPr | Cyclobutyl-C(O)— | 537.1 | 537 |
| 2-17 | 4-Me | iPr | PhCH₂CH₂C(O)— | 587.2 | 587 |
| 2-18 | 4-Me | iPr | PrC(O)— | 525.1 | 525 |
| 2-19 | 4-Me | iPr | Ph(CH₂)₃C(O)— | 601.2 | 601 |
| 2-20 | 4-Me | iPr | Ph(CH₂)₄C(O)— | 615.3 | 615 |
| 2-21 | 4-CF₃ | iBu | MeSO₂— | 601.0 | |

### Example 3

### 2-{4-[1-(1-aminoacetyl)-3-(4-chlorophenyl)-1-pyrrolidinecarbonyl]-1-piperazinyl}-1-[1S-amino-3-methylbutyl]-5-trifluoromethylbenzene

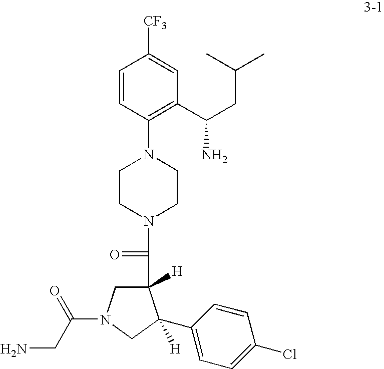

### Step 3A. 2-{4-[1-(1-aminoacetyl)-3-(4-chlorophenyl)-1-pyrrolidinecarbonyl]-1-piperazinyl}-1-[1S-amino-3-methylbutyl]-5-trifluoromethylbenzene

2-{4-[3-(4-chlorophenyl)-1-pyrrolidinecarbonyl]-1-piperazinyl}-1-[1S-(S-t-butanesulfinamido)-3-methylbutyl]-5-trifluoromethylbenzene (1e, 62.7 mg, 0.1 mmol) was dissolved in dichloromethane (0.5 mL) along with HOBt (13.5 mg, 0.1 mmol) and Boc-glycine (17.5 mg, 0.1 mmol). The reaction mixture was allowed to stir at room temperature for 10 minutes then EDC (19.2 mg, 0.1 mmol) was added. The reaction was stirred at room temperature for an additional 8 hours and was washed with saturated NaHCO₃ solution (2 mL). The organic layer was separated and evaporated to dryness under a stream of nitrogen. The residue was dissolved in 2 mL of (1:1) TFA/DCM and stirred at room temperature for 1 hour. Solvent was then removed by evaporating under a stream of nitrogen and the residue was purified by preparative HPLC. The compound 3-1 was recovered as the TFA salt in 54% overall yield from the 2-fluoro-5-trifluoromethylbenzaldehyde of step 1A. MS: calc. for C₂₉H₃₇ClF₃N₅O₂: 579.26; Found: 580 (MH+); retention time: 7.43 minutes; Method info: APCI positive ion scan 100-1000 Frag V=80; 95% 0.05% TFA/H₂O to 95% ACN/0.05% TFA over 13 min, 15.5 min run, ODS-AQ column.

By the above procedures, the compounds of the following Table 3 were prepared.

TABLE 3

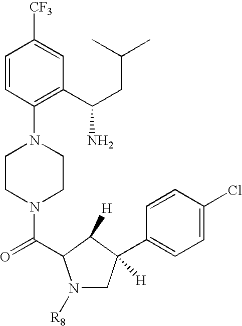

| Cpd | R₈ | M.W. | (MH⁺) |
|---|---|---|---|
| 3-1 | NH₂CH₂C(O)— | 580.09 | 581 |
| 3-2 | PhC(O)— | 627.15 | 628 |

### Example 4

### 2-{4-[1-phenyl-3-(4-chlorophenyl)-1-pyrrolidinecarbonyl]-1-piperazinyl}-1-[1S-amino-3-methylbutyl]-5-trifluoromethylbenzene

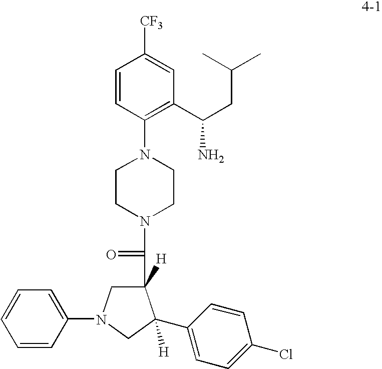

### Step 4A. 2-{4-[1-phenyl-3-(4-chlorophenyl)-1-pyrrolidinecarbonyl]-1-piperazinyl}-1-[1S-amino-3-methylbutyl]-5-trifluoromethylbenzene 4-1

2-{4-[3-(4-Chlorophenyl)-1-pyrrolidinecarbonyl]-1-piperazinyl}1-[1S-(S-t-butanesulfinamido)-3-methylbutyl]-5-trifluoromethylbenzene (1e, 62.7 mg, 0.1 mmol) was placed in a capped reaction vial along with CsCO₃ (45.6 mg, 0.14 mmol), Pd(OAc)₂ (2.7 mg, 0.004mmol), (+)-BINAP (3.74 mg, 0.006 mmol), bromobenzene (9 uL, 0.085 mmol), and 1,4-dioxane (0.4 mL). The reaction was allowed to stir under nitrogen atmosphere at 100° C. for 24 hours then another portion of CsCO₃ (45.6 mg, 0.14 mmol), Pd(OAc)₂ (2.7 mg, 0.004 mmol), and (+)-BINAP (3.74 mg, 0.006 mmol) was added. The reaction was continued heating at 100° C. undernitrogen atmosphere for an additional 24 hours. The mixture was then cooled to room temperature, diluted with ether (2 mL), and filtered. The organic layer was concentrated under a stream of nitrogen and the residue was dissolved in 2 mL of MeOH and 0.5 mL of 2N HCl in ether was added. The reaction was stirred at room temperature for 1 hour then solvent was removed by evaporating under a stream of nitrogen and the crude product was purified by preparative HPLC. The compound 4-1 was recovered as the TFA salt in 7.4% overall yield from the 2-fluoro-5-trifluoromethylbenzaldehyde of Step 1A. MS: calc. for C₃₃H₃₈ClF₃N₄O: 598.27; Found: 599 (MH⁺); retention time: 12.25 minutes; Method info: APCI positive ion scan 100-1000 Frag V=80; 95% 0.05% TFA/H₂O to 95% ACN/0.05% TFA over 13 min, 15.5 min run, ODS-AQ column.

By the above procedures, the compound of the following Table 4 was prepared.

TABLE 4

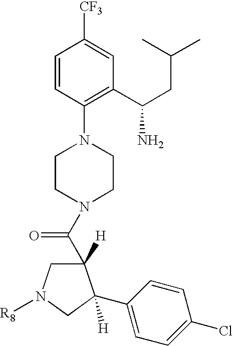

| Cpd | R₈ | M.W. | (MH⁺) |
|---|---|---|---|
| 4-1 | Ph | 599.14 | 600 |

### Example 5

### 4-[4-(trifluoromethyl)-2-(1S-glycineamido-3-methylbutyl)phenyl]-1-[1-[1-isopropyl-3-(4-chlorophenyl)pyrrolidinecarbonyl]piperazine

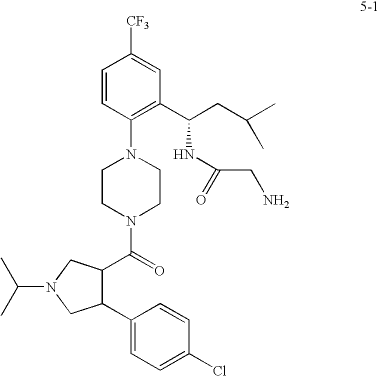

### Step SA: Compound 5-1

Pyrrolidine 1-1 (62.7 mg, 0.1 mmol) was dissolved in dichloromethane (0.5 mL) along with HOBt (13.5 mg, 0.1 mmol) and Boc-glycine (17.5 mg, 0.1 mmol). The reaction mixture was allowed to stir at room temperature for 10 minutes then EDC (19.2 mg, 0.1 mmol) was added. The reaction was stirred at room temperature for an additional 8 hours then was washed with saturated NaHCO₃ solution (2 mL). The organic layer was separated and evaporated to dryness under a stream of nitrogen. The residue was dissolved in 2 mL of (1:1) TFA/DCM and stirred at room temperature for 1 hour. Solvent was then removed by evaporating under a stream of nitrogen and the crude product was purified by preparative HPLC. Compound 5-1 was recovered as the TFA salt in 62% overall yield from compound 1-1. MS: calc. for C₃₂H₄₃ClF₃N₅O₂: 621; Found: 622 (MH⁺); retention time: 7.605 minutes; Method info: Electrospray positive (ES+) ionization, MW scan range 150-966 m/z, Detector Voltage 650V, Probe temp. 325C; 21.85 min. run with gradient of 10% Acetonitrile (w/0.035% TFA), 90% H₂O (w/0.05% TFA) to 95% ACN (w/0.035% TFA), 5% H₂O (w/0.05% TFA) over 18.36 min., flow rate 2.5 ml/min.; 4.6×100 mm, ODS-O/B, 5 micron, 120 Angstrom column, run at ambient temperature.

By the above procedures, the compounds of the following Table 5 were prepared.

TABLE 5

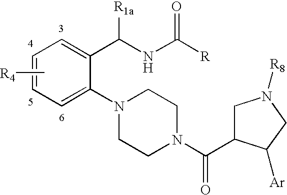

| Cpd | R₄ | R₁ₐ | R | Ar | R₈ | MW | (MH⁺) |
|---|---|---|---|---|---|---|---|
| 5-1 | 4-CF₃ | iBu | —CH₂NH₂ | 4-Cl-Ph- | iPr | 622.2 | 622 |
| 5-2 | 4-CF₃ | iBu | —CH₂NHMe | 4-Cl-Ph- | iPr | 636.2 | 637 |
| 5-3 | 4-CF₃ | iBu | —CH₂NMe₂ | 4-Cl-Ph- | iPr | 650.2 | 650 |
| 5-4 | 4-CF₃ | iBu | (R)-CH(Me)NH₂ | 4-Cl-Ph- | iPr | 636.2 | 636 |
| 5-5 | 4-CF₃ | iBu | (S)-CH(Me)NH₂ | 4-Cl-Ph- | iPr | 636.2 | 636 |
| 5-6 | 4-CF₃ | iBu | —CH₂CH₂NH₂ | 4-Cl-Ph- | iPr | 636.2 | 636 |
| 5-7 | 4-CF₃ | iBu | —CH₂CH₂NMe₂ | 4-Cl-Ph- | iPr | 664.3 | 664 |
| 5-8 | 4-CF₃ | iBu | 3-azetidinyl | 4-Cl-Ph- | iPr | 648.2 | 648 |
| 5-9 | 4-CF₃ | iBu | 2-pyrrolidinyl | 4-Cl-Ph- | iPr | 662.2 | 662 |
| 5-10 | 6-F | iPr | —CH₂NHMe | 4-Cl-Ph- | iPr | 572.2 | 572 |
| 5-11 | 6-F | iPr | (R)-CH(Me)NH₂ | 4-Cl-Ph- | iPr | 572.2 | 572 |
| 5-12 | 6-F | iPr | —CH₂CH₂NH₂ | 4-Cl-Ph- | iPr | 572.2 | 572 |
| 5-13 | 6-F | iPr | —CH₂CH₂NH₂ | 2,4-di-Cl- | iPr | 606.6 | 606 |
| | | | | Ph- | | | |
| 5-14 | 6-F | iPr | —CH₂NHMe | 2,4-di-Cl- | iPr | 606.6 | 606 |
| | | | | Ph | | | |
| 5-15 | 6-F | iPr | —CH₂CH₂NHMe | 2,4-di-Cl- | iPr | 620.6 | 620 |
| | | | | Ph | | | |
| 5-16 | 4-CF₃ | iBu | —CH₂CH₂NMe₂ | 2,4-di-Cl- | iPr | 698.7 | 698 |
| | | | | Ph | | | |
| 5-17 | 4-CF₃ | iBu | —CH₂CH₂NH₂ | 2,4-di-Cl- | iPr | 670.6 | 670 |
| | | | | Ph- | | | |
| 5-18 | 4-CF₃ | iBu | —CH₂NHMe | 2,4-di-Cl- | iPr | 670.6 | 670 |
| | | | | Ph- | | | |
| 5-19 | 4-CF₃ | iBu | —CH₂CH₂NHMe | 2,4-di-Cl- | iPr | 684.7 | 684 |
| | | | | Ph- | | | |
| 5-20 | 4-Me | iPr | —CH₂NMe₂ | 4-Cl-Ph- | iPr | 582.2 | 582 |
| 5-21 | 4-Me | iPr | —CH₂CH₂NMe₂ | 4-Cl-Ph- | iPr | 596.3 | 596 |
| 5-22 | 4-Cl | iBu | —CH₂CH₂NMe₂ | 4-Cl-Ph- | cyclopentyl | 656.7 | 656 |
| 5-23 | 4-Cl | iBu | —CH₂CH₂NMe₂ | 4-Cl-Ph- | Bn | 678.7 | 678 |
| 5-24 | 4-Cl | iBu | —CH₂CH₂NMe₂ | 4-Cl-Ph- | cyclohexyl | 670.8 | 670 |
| 5-25 | 6-F | iPr | —CH₂CH₂NMe₂ | 4-Cl-Ph- | cyclopentyl | 626.3 | 626 |
| 5-26 | 6-F | iPr | —CH₂CH₂NMe₂ | 4-Cl-Ph- | Bn | 648.3 | 648 |
| 5-27 | 6-F | iPr | —CH₂CH₂NMe₂ | 3,4,5-tri-F- | iPr | 619.7 | 620 |
| | | | | Ph | | | |
| 5-28 | 6-F | iPr | —CH₂CH₂NMe₂ | 3-F-Ph- | iPr | 583.8 | 584 |
| 5-29 | 6-F | iPr | —CH₂CH₂NMe₂ | 3-Cl-Ph- | iPr | 600.2 | 600 |
| 5-30 | 6-F | iPr | —CH₂CH₂NMe₂ | 3,4-di-F-Ph- | iPr | 601.8 | 602 |
| 5-31 | 6-F | iPr | —CH₂CH₂NMe₂ | 3-Me-Ph | iPr | 579.8 | 580 |
| 5-32 | 6-F | iPr | —CH₂CH₂NMe₂ | 3-MeO-Ph- | iPr | 595.8 | 596 |
| 5-33 | 6-F | iPr | —CH₂CH₂NMe₂ | 4-Cl-Ph- | Bn | 648.3 | 648 |
| 5-34 | 6-F | iPr | —CH₂CH₂NMe₂ | 4-Cl-Ph- | Bn | 648.3 | 648 |
| 5-35 | 6-F | iPr | —CH₂CH₂NMe₂ | 2-F-4-CF₃- | iPr | 651.8 | 652 |
| | | | | Ph- | | | |
| 5-36 | 6-F | iPr | -CH₂CH₂NMe₂ | 2,5-di-F-Ph- | iPr | 601.8 | 602 |
| 5-37 | 6-F | iPr | -CH₂CH₂NMe₂ | 3-F-4-CF₃- | iPr | 651.8 | 652 |
| | | | | Ph | | | |
| 5-38 | 6-F | iPr | —CH₂CH₂NMe₂ | 4-F-3-Cl-Ph | iPr | 618.2 | 618 |
| 5-39 | 6-F | iPr | —CH₂CH₂NMe₂ | 3,5-di-F-Ph- | iPr | 601.8 | 602 |
| 5-40 | 4-Me | iPr | Me | 4-Cl-Ph- | iPr | 539.2 | 539 |
| 5-41 | 4-Me | iPr | —CH₂CH₂NMe₂ | 4-Cl-Ph- | H | 554.2 | 554 |
| 5-42 | 4-Me | iPr | —CH₂CH₂NH₂ | 4-Cl-Ph- | 4- | 610.2 | 610 |
| | | | | | tetrahydropyranyl | | |
| 5-43 | 4-Me | iPr | —CH₂NH₂ | 4-Cl-Ph- | 4- | 596.2 | 596 |
| | | | | | tetrahydropyranyl | | |
| 5-44 | 4-Me | iPr | —CH₂NHMe | 4-Cl-Ph- | 4- | 610.2 | 610 |
| | | | | | tetrahydropyranyl | | |
| 5-45 | 4-Me | iPr | (S)-CH(Me)NH₂ | 4-Cl-Ph- | 4- | 610.2 | 610 |
| | | | | | tetrahydropyranyl | | |
| 5-46 | 4-Me | iPr | (R)-CH(Me)NH₂ | 4-Cl-Ph- | 4- | 610.2 | 610 |
| | | | | | tetrahydropyranyl | | |
| 5-47 | 6-F | iPr | —CH₂CH₂NMe₂ | 4-Cl-Ph- | cyclohexyl | 640.3 | 640 |
| 5-48 | 4-Me | iPr | —CH₂CH₂NMe₂ | 4-Cl-Ph- | FCH₂CH(Me)— | 614.2 | 614 |
| 5-49 | 4-Me | iPr | —CH₂CH₂NMe₂ | 4-Cl-Ph- | CF₃CH₂CH(Me)— | 650.2 | 650 |

### Example 6

### 4-[2-(1S-{3-piperidyl}amino-ethyl)phenyl]-1-[1-isopropyl-3-(4-chlorophenyl)pyrrolidinecarbonyl]piperazine

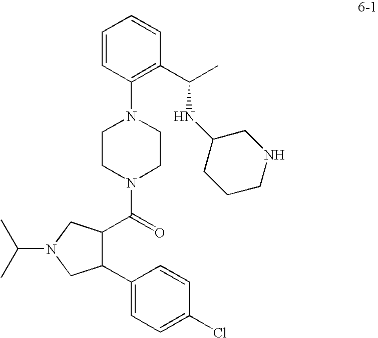

### Step 6A: Compound 6-1

Piperidine 6a (0.93 g, 3.07 mmol, synthesized according to the procedure of Step 1A from 2′-fluoroacetophenone and 1-BOC-piperazine) was dissolved in (1:1) TFA/DCM (14 mL) and was stirred at room temperature for 30 minutes. The reaction mixture was then diluted with dichloromethane (30 mL) and washed with saturated NaHCO₃ solution (3×50 mL) until excess TFA was neutralized. The organic layer was then washed once with saturated NaCl solution (50 mL), dried over anhydrous MgSO₄, filtered, and evaporated to dryness in vacuo. The crude material was then added to a mixture containing 1-[(tert-butyl)oxycarbonyl]-4-(4-chlorophenyl)pyrrolidine-3-carboxylic acid in DMF (13 mL) with HBTU (1.16g, 3.07 mmol) and DIEA (1.1 mL, 6.14 mmol) that had been stirring at room temperature for 1 hour. The reaction was stirred at room temperature for an additional 4 hours. The reaction mixture was diluted with ethyl acetate (50 mL), then was washed with NaHCO₃ solution (3×50 mL) and saturated NaCl solution (50 mL). The organic layer was separated, dried over anhydrous MgSO₄, filtered, and evaporated to dryness in vacuo. The crude coupling product was purified by column chromatography on silica using 40% ethyl acetate/hexanes as the eluent (R_{f}=0.3). Compound 6b was recovered in 81% yield (1.03 g) as an off-white solid.

### Step 6B

Pyrrolidine 6b (1.03 g, 2.49 mmol) was dissolved in (1:1) TFA/DCM (20 mL) and stirred at room temperature for 1 hour. The reaction mixture was then diluted with dichloromethane (50 mL) and washed with saturated NaHCO₃ solution (3×75 mL) until excess TFA was neutralized. The organic layer was then washed once with saturated NaCl solution (75 mL), dried over anhydrous MgSO₄, filtered, and evaporated to dryness in vacuo. The crude product was dissolved in 1,2-dichloroethane (12.5 mL) along with acetone (1.1 mL, 15 mmol), NaBH(OAc)₃ (0.79 g, 3.74 mmol), and AcOH (145 μl, 2.49 mmol). The reaction mixture was allowed to stir at room temperature for 8 hours then diluted with dichloromethane (20 mL) and washed with saturated NaHCO₃ solution (3×50 mL). The organic layer was then washed once with saturated NaCl solution (50 mL), dried over anhydrous MgSO₄, filtered, and evaporated to dryness in vacuo. Compound 6c was recovered in 93% yield (1.03g) as an off-white solid without further purification.

### Step 6C

Compound 6c (45 mg, 0.1 mmol) was dissolved in (1:1) 1,2-dichloroethane (0.5 mL)/THF (0.5 mL) along with (±)-3-amino-1-N-Boc-piperidine (20 mg, 0.1 mmol), NaBH(OAc)₃ (30 mg, 0.14 mmol), and AcOH (17.1 ul, 0.3 mmol). The reaction mixture was stirred at 55° C. for 12 hours then was diluted with dichloromethane (3 mL) and was washed with saturated NaHCO₃ solution (3×5 mL). The organic layer was separated and evaporated to dryness under a stream of nitrogen. The residue was dissolved in 2mL of (1:1) TFA/DCM and stirred at room temperature for 1 hour. Solvent was then removed by evaporating under a stream of nitrogen and the crude product was purified by preparative HPLC. Compound 6-1 was recovered as the TFA salt in 29% overall yield. MS: calc. for C₃]H₄₄ClN₅O: 537; Found: 538 (MH⁺); retention time: 3.39 minutes; Method info: APCI positive ion scan 100-1000 Frag V=80; 95% 0.05% TFA/H₂O to 95% ACN/0.05% TFA over 13 min, 15.5 min run, SynergiMAX-RP column 2×50 mm.

By the above procedures, the compounds of the following Table 6 were prepared.

TABLE 6

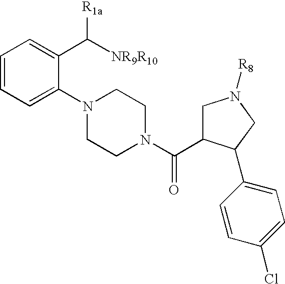

| Cpd | R₁ₐ | NR₉R₁₀ | R₈ | (MH⁺) | MW |
|---|---|---|---|---|---|
| 6-1 | Me | 3-piperidinylNH | iPr | 538 | 538.2 |
| 6-2 | Me | 2-furanylCH₂NH | iPr | 535 | 535.1 |
| 6-3 | Me | 1-piperidinylCH₂CH₂NH | iPr | 566 | 566.2 |
| 6-4 | Me | cyclopropylNH | iPr | 495 | 495.1 |
| 6-5 | Me | 2-methoxycyclohexylNH | iPr | 553 | 553.2 |
| 6-6 | Me | cyclobutylNH | iPr | 509 | 509.1 |
| 6-7 | Me | cyclopentylNH | iPr | 523 | 523.2 |
| 6-8 | Me | iPrNH | iPr | 497 | 497.1 |
| 6-9 | Me | MeOCH₂CH₂NH | iPr | 513 | 513.1 |
| 6-10 | Me | 4-F-PhCH₂CH₂NH | iPr | 577 | 577.2 |
| 6-11 | Me | 2-aminophenylNH | iPr | 546 | 546.2 |
| 6-12 | Me | cyclohexylNH | iPr | 537 | 537.2 |
| 6-13 | H | 2-aminocyclohexylNH | iPr | 538 | 538.2 |
| 6-14 | H | 2-aminophenylNH | iPr | 532 | 532.1 |
| 6-15 | H | (R)-3-amino-1-pyrrolidinyl | iPr | 510 | 510.1 |
| 6-16 | H | (S)-3-amino-1-pyrrolidinyl | iPr | 510 | 510.1 |
| 6-17 | H | 3-methylamino-1-pyrrolidinyl | iPr | 524 | 524.1 |
| 6-18 | H | 2-aminoethylNH | iPr | 484 | 484.1 |
| 6-19 | H | 2-methylaminoethylNH | iPr | 498 | 498.1 |
| 6-20 | H | 3-piperidinylNH | iPr | 524 | 524.1 |
| 6-21 | H | 2-pyrrolidinylCH₂NH | iPr | 524 | 524.1 |
| 6-22 | H | (S)-3-pyrrolidinylNH | iPr | 510 | 510.1 |
| 6-23 | H | 3-PyCH | iPr | 518 | 518.1 |
| 6-24 | H | 4-amino-1-piperidinylNH | iPr | 524 | 524.1 |
| 6-25 | H | 4-piperidinylNH | iPr | 524 | 524.1 |
| 6-26 | H | 3-azetidinylCH₂NH | iPr | 510 | 510.1 |
| 6-27 | H | 3-azetidinylNH | iPr | 496 | 496.1 |
| 6-28 | H | (R)-3-pyrrolidinyl | iPr | 510 | 510.1 |
| 6-29 | H | trans-2-aminocyclohexylNH | iPr | 538 | 538.2 |
| 6-30 | H | MeOCH₂CH₂NMe | iPr | 513 | 513.1 |
| 6-31 | H | Me₂N | iPr | 469 | 469.1 |
| 6-32 | H | Et₂N | iPr | 497 | 497.1 |
| 6-33 | H | pyrrolidinyl | iPr | 495 | 495.1 |
| 6-34 | H | piperidinyl | iPr | 509 | 509.1 |
| 6-35 | H | piperazinyl | iPr | 510 | 510.1 |
| 6-36 | H | 4-methylpiperazinyl | iPr | 524 | 524.1 |
| 6-37 | H | 3-pyrrolidinylCH₂NH | iPr | 524 | 524.1 |
| 6-38 | H | imidazolyl | iPr | 492 | 492.1 |
| 6-39 | H | 2-methylimidazolyl | iPr | 506 | 506.1 |
| 6-40 | H | 5-methylimidazolyl | iPr | 506 | 506.1 |
| 6-41 | H | 1,2,4-triazol-4-yl | iPr | 493 | 493.1 |
| 6-42 | H | 1,2,4-triazol-1-yl | iPr | 493 | 493.1 |
| 6-43 | H | 1,2,3-triazol-1-yl | iPr | 493 | 493.1 |
| 6-44 | H | 1-pyrazolyl | iPr | 492 | 492.1 |
| 6-45 | H | iBu | iPr | 497 | 497.1 |
| 6-46 | H | (iBu)₂N | iPr | 553 | 553.2 |

### Example 7

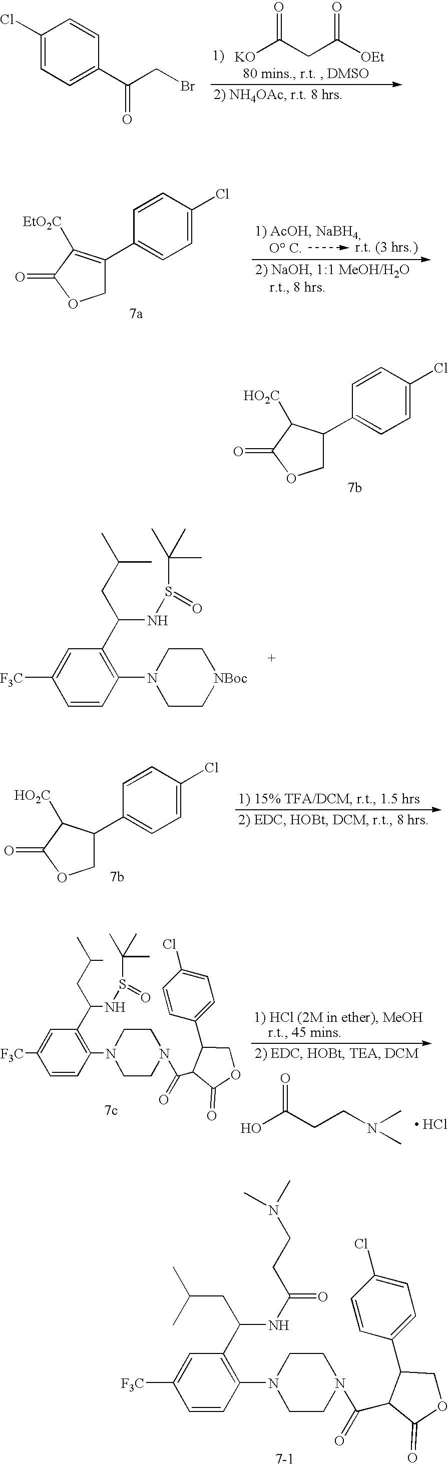

### Step 7A: 4-Chlorophenyl Lactone 7b

4-Chlorophenacylbromide (5 g, 21.4 mmol) was added slowly over 15 minutes under nitrogen atmosphere with stirring to a mixture of malonic acid monoethylester potassium salt (4.4 g, 25.7 mmol) in DMSO (20.6 mL). The reaction mixture was allowed to stir at room temperature for 80 minutes, then ammonium acetate (1.3 g, 16.8 mmol) was added in one portion. After 8 hours at room temperature, the unsaturated lactone 7a was formed (checked by IR and GC). To the reaction mixture, acetic acid (3.6 mL, 63.7 mmol) was added and the reaction mixture was cooled to 0° C. and sodium borohydride (0.63 g, 16.7 mmol) was added over 25 minutes followed by stirring at room temperature for 3 hours. After the reaction was complete, ice water was added to the reaction flask and the crude product was isolated by partitioning between ethyl acetate/water. The organic phase was collected and solvent was evaporated in vacuo. The crude material was then added to a reaction flask containing sodium hydroxide (1.5 g, 37.9 mmol) in 1:1 MeOH/H₂O (66 mL) and stirred at room temperature for 8 hours. After 8 hours, methanol was removed in vacuo and to the residue was added 10% sodium hydroxide solution (20 mL), and the aqueous layer was washed with ethyl acetate (2×25 mL). The water layer was isolated and acidified under ice-cooling to pH=1-2 with concentrated HCl and the white precipitate was collected by filtration. The precipitate was dissolved in ethyl acetate and added to hexanes. The resulting white solid was collected and dried under high vacuum to give 1.4 g of compound 7b (21% yield). ¹H NMR (CDCl₃) 3.88-3.19 (d, 1H, CH), 4.11-4.286 (m, 2H, CH₂), 4.67-4.72 (t, 1H, CH), 7.37 (s, 4H, ArH).

### Step 7B: 4-Trifluoromethylphenyl Lactone 7c

2-[4-(tert-Butoxycarbonyl)-1-piperazinyl]-1-[1S-(S-t-butanesulfinamido)-3-methylbutyl]-5-trifluoromethylbenzene 1c (4.73 g, 9.1 mmol) was dissolved in 15% TFA/DCM (35 mL) and stirred at room temperature for 1.5 hours (reaction was monitored by TLC). The reaction mixture was then diluted with dichloromethane (60 mL) and quenched by slowly adding to a saturated solution of potassium carbonate (150 mL). The organic layer was then isolated and washed with saturated NaHCO₃ solution (2×100 mL) followed by washing with saturated NaCl solution (100 mL). The organic layer was isolated, dried over anhydrous MgSO₄, filtered, and evaporated to dryness in vacuo. 2-[1-piperazinyl]-1-[1S-(S-t-butanesulfinamido)-3-methylbutyl]-5-trifluoromethylbenzene 1c.1 was recovered in quantitative yield and an aliquot was used for the next step without any further purification. The deprotected piperazine intermediate (1.26 g, 3 mmol) was dissolved in DCM (15 mL) along with HOBt (0.41 g, 3 mmol) and Cl-phenyl lactone acid 7b (0.72 g, 3 mmol). The reaction mixture was allowed to stir at room temperature for 10 minutes then EDC (0.58 g, 3 mmol) was added. The reaction was then allowed to stir at room temperature for an additional 8 hours. After 8 hours, the reaction mixture was diluted with dichloromethane (20 mL) then washed with saturated NaHCO₃ (3×50 mL) and saturated NaCl (50 mL). The organic layer was collected, dried over anhydrous MgSO₄, filtered, and evaporated to dryness under vacuum. Compound 7c was recovered in 11% yield (0.21 g, 0.32 mmol) after purification by column chromatography on silica using 50% ethyl acetate/hexanes as the eluent (R_{f}═0.3, two spots corresponding to cis and trans isomers). MS: calc. for C₃₁H₃₉ClF₃N₃O₄S: 641.23; Found: 642 (MH⁺); retention time: 3.246 minutes; Method info: APCI positive ion scan 100-1000 Frag V=80; 95% 0.05% TFA/H₂O to 95% ACN/0.05% TFA over 2 min, 3.4 min run, ODS-AQ column.

### Step 7C: 4-Trifluoromethylphenyl Piperazine 7-1

Trifluoromethylphenyl sulfinamide 7c (0.21 g, 0.32 mmol) was dissolved in MeOH (3.2 mL) and HCl (2M in ether, 208 μL, 0.42 mmol) was added to the reaction vial. The reaction mixture was allowed to stir at room temperature for 45 minutes (monitored by TLC). Nitrogen gas was then bubbled through the reaction mixture to evaporate residual HCl then the remaining solvent was removed in vacuo. The residue was dissolved in dichloromethane (10 mL), washed with saturated NaHCO₃ (3×20 mL) and saturated NaCl (20 mL). The organic layer was collected, dried over anhydrous MgSO₄, filtered, and evaporated to dryness under vacuum. A portion of the deprotected intermediate (53.8 mg, 0.1 mmol) was then dissolved in dichloromethane (0.5 mL) along with 3-dimethylaminopropionic acid hydrochloride (15.3 mg, 0.1 mmol), and triethylamine (14 μL, 0.1 mmol). The reaction mixture was allowed to stir at room temperature for 5 minutes then HOBt (13.5 mg, 0.1 mmol) was added. After another 5 minutes, EDC (19.2 mg, 0.1 mmol) was added to the reaction mixture and stirring was continued at room temperature for an additional 8 hours. The reaction mixture was then diluted with dichloromethane (3 mL) and washed with saturated NaHCO₃ (3×10 mL) followed by saturated NaCl solution(10 mL). The organic layer was collected and evaporated to dryness under a stream of nitrogen. The crude product was purified by preparative HPLC. The compound 7-1 was recovered as the TFA salt in 44% yield. MS: calc. for C₃₂H₄₀ClF₃N₄O₄: 636.2; Found: 637 (MH⁺); retention time: 7.6 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 7 were prepared.

TABLE 7

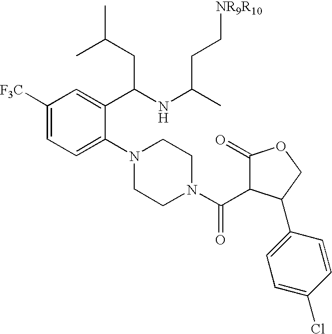

| Cpd | NR₉R₁₀ | MW | (MH⁺) |
|---|---|---|---|
| 7-1 | NMe₂ | 636.1 | 637 |
| 7-2 | NH₂ | 608.1 | 609 |
| 7.3 | 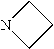 | 648.1 | 649 |

### Example 8

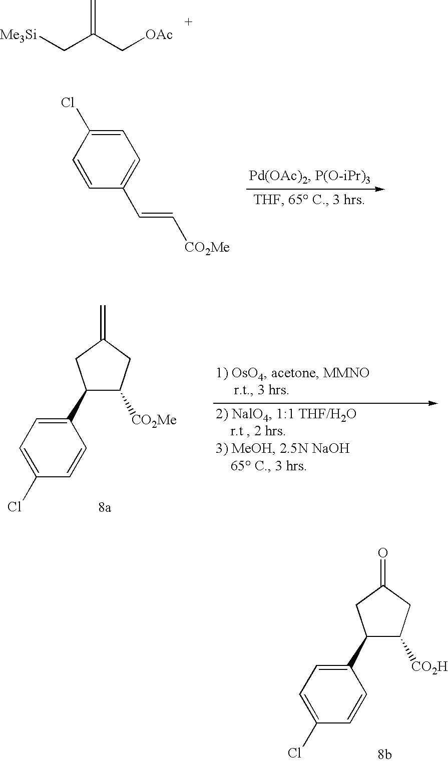

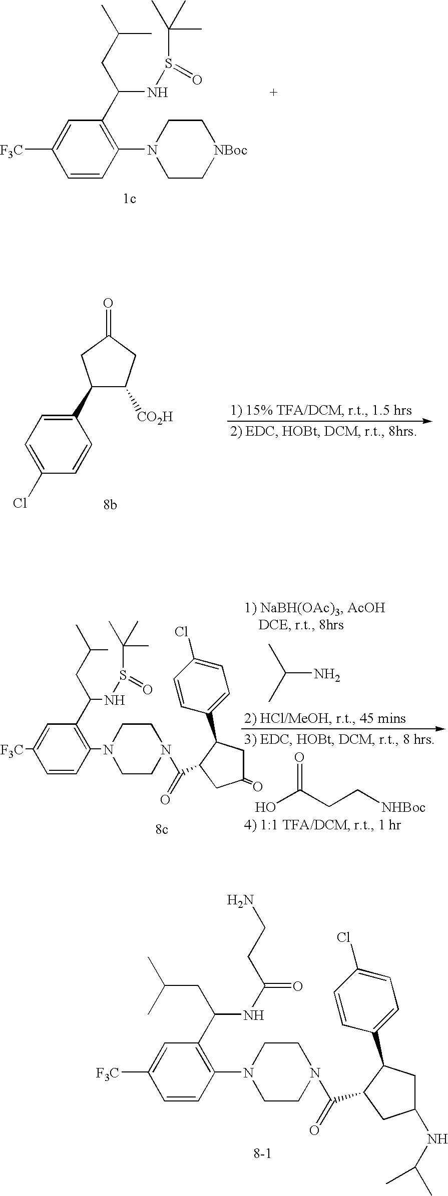

### Step 8A: Cl-Phenylcyclopentyl Ester 8a

To an oven dried flask, methyl 4-chlorocinnamate (4 g, 20.5 mmol) was dissolved in THF (41 mL) along with palladium acetate (276 mg, 1.23 mmol). Air was removed from the reaction flask by vacuum and flushing with nitrogen (repeated three times). The reaction flask was stirred under nitrogen atmosphere and 2-[(trimethylsilyl)methyl]-2-propen-1-yl acetate (5.5 mL, 26.8 mmol) was added followed by triisopropyl phosphite (1.4 mL, 6.2 mmol). The reaction mixture was refluxed for 3 hours under nitrogen atmosphere then cooled to room temperature. The reaction mixture was then transferred to a separatory funnel and partitioned between water (100 mL) and ether (100 mL). The organic layer was washed with water (100 mL), saturated NaCl solution (100 mL), dried over MgSO₄, and filtered. Solvent was removed in vacuo and 8a was recovered in 96% yield (4.95 g, 19.7mmol) after purification by column chromatography on silica using 10% ethyl acetate/hexanes as the eluent (R_{f}═0.3). MS: calc. for C₁₄H₁₅ClO₂: 250.08; Found: GC-MS m/z 250 (MH⁺).

### Step 8B: Cyclopentanone 8b

Cl-Phenylcyclopentyl ester 8a (2 g, 8 mmol) was added to the reaction flask along with acetone (14.4 mL). To the reaction mixture, 4-methylmorpholine N-oxide (1.12 g, 9.6 mmol) dissolved in water (3 mL) was added followed by osmium teteroxide (106 mg, 0.42 mmol). The reaction mixture was stirred at room temperature for 3 hours then was quenched with 10% sodium bisulfite and partitioned between water and ethyl acetate. The organic layer was washed with water, collected, dried over MgSO₄, filtered, and evaporated to dryness in vacuo. The residue was redissolved in 1:1 THF/H₂O (19.2 mL) and sodium periodate (2 g, 9.6 mmol) along with an additional 4.8 mL of THF was added. The reaction mixture was allowed to stir at room temperature for 2 hours at which time starting material had been completely consumed (by TLC). The reaction mixture was added to water and extracted with ethyl acetate. The organic layer was collected, dried over MgSO₄, filtered, and evaporated to dryness in vacuo. The residual oil was used for the next step without purification. The residue was dissolved in methanol (65 mL) and aqueous sodium hydroxide (13.5 mL, 2.5M, 33.8 mmol) was added. The reaction mixture was allowed to stir at 65° C. for 3 hours. The reaction mixture was then cooled to room temperature and partitioned between methylene chloride and water. The organic layer was separated, washed with IN HCl followed by saturated NaCl solution. The organic layer was then dried over MgSO₄, filtered, and evaporated to dryness in vacuo. The crude solid was recrystallized from ethyl acetate/hexanes to give 8b in 68% yield (1.3 g) over 3 steps.

### Step 8C: Cyclopentanone 8c

2-[4-(tert-Butoxycarbonyl)-1-piperazinyl]-1-[1S-(S-t-butanesulfinamido)-3-methylbutyl]-5-trifluoromethylbenzene 1c (2.13 g, 4.1 mmol) was dissolved in 15% TFA/DCM (15.8 mL) and stirred at room temperature for 1.5 hours (reaction was monitored by TLC). The reaction mixture was then diluted with dichloromethane (20 mL) and quenched by slowly adding to a saturated solution of potassium carbonate (60 mL). The organic layer was then isolated and washed with saturated NaHCO₃ solution (2×50 mL) followed by washing with saturated NaCl solution (50 mL). The organic layer was isolated, dried over anhydrous MgSO₄, filtered, and evaporated to dryness in vacuo. The crude deprotected intermediate was recovered in quantitative yield and was used for the next step without any further purification. The deprotected piperazine intermediate (1.7 g, 4.1 mmol) was dissolved in DCM (20 mL) along with HOBt (0.55 g, 4.1 mmol) and Cl-PhenylKeto Acid 8b (0.98 g, 4.1 mmol). The reaction mixture was allowed to stir at room temperature for 10 minutes then EDC (0.79 g, 4.1 mmol) was added. The reaction was then allowed to stir at room temperature for an additional 8 hours. After 8 hours, the reaction mixture was washed with saturated NaHCO₃ (3×60 mL) and saturated NaCl (60 mL). The organic layer was collected, dried over anhydrous MgSO₄, filtered, and evaporated to dryness under vacuum. Compound 8c was recovered in 19% yield (0.49 g, 0.76 mmol) after purification by column chromatography on silica using 75% ethyl acetate/hexanes as the eluent (R_{f}=0.3). MS: calc. for C₃₂H₄₁ClF₃N₃O₃S: 639.25; Found: 640 (MH⁺); retention time: 3.244 minutes; Method info: APCI positive ion scan 100-1000 Frag V=80; 95% 0.05% TFA/H₂O to 95% ACN/0.05% TFA over 2 min, 3.4 min run, ODS-AQ column.

### Step 8D: Isopropylcyclopentyl amine 8-1

Cyclopentanone 8c (128 mg, 0.2 mmol) was dissolved in DCE (1 mL) along with isopropylamine (17 uL, 0.2 mmol), acetic acid (11.5 uL, 0.2 mmol), and sodium triacetoxyborohydride (59.3 mg, 0.28 mmol). The reaction was allowed to stir at room temperature for 8 hours then diluted with dichloromethane and washed with saturated NaHCO₃ solution (3×5 mL) followed by saturated NaCl solution (5 mL). The organic layer was isolated and solvent was removed in vacuo. The residue was dissolved in methanol (2 mL) along with HCl (250 μL, 2M in ether, 0.5 mmol) and stirred at room temperature for 45 minutes. The reaction mixture was then evaporated to dryness under a stream on nitrogen, redissolved in dichloromethane, and washed with saturated NaHCO₃ solution (3×5 mL) followed by saturated NaCl solution (5 mL). The organic layer was evaporated to dryness in vacuo and an aliquot (approximately half, 0.1 mmol) was used without further purification for the next step. The crude aliquot was dissolved in dichloromethane (0.5 mL) along with HOBt (13.5 mg, 0.1 mmol) and Boc-β-alanine (18.9 mg, 0.1 mmol). The reaction mixture was allowed to stir at room temperature for 10 minutes then EDC (19.2 mg, 0.1 mmol) was added. The reaction was then allowed to stir at room temperature for an additional 8 hours. After 8 hours, the reaction mixture was diluted with dichloromethane (3 mL) and washed with saturated NaHCO₃ (2×5 mL). The organic layer was collected and evaporated to dryness under vacuum. The residue was dissolved in 1:1 TFA/DCM (1 mL) and stirred at room temperature for 1 hour. The reaction mixture was then evaporated to dryness under a stream on nitrogen and purified by preparative HPLC. The compound 8-1 was recovered as the TFA salt in 43% yield. MS: calc. for C₃₄H₄₇ClF₃N₅O₂: 649.3; Found: 650 (MH⁺); retention time: 5.704 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 8 were prepared.

TABLE 8

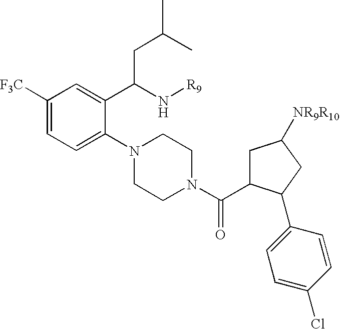

| Cpd | NR₉R₁₀ | R₉ | MW | (MH⁺) |
|---|---|---|---|---|
| 8-1 | NHPr-i | C(O)CH₂CH₂NH₂ | 635.2 | 636 |
| 8-2 | NMe₂ | C(O)CH₂CH₂NH₂ | 649.2 | 650 |
| 8-3 | NHPr-i | H | 578.1 | 579 |
| 8-4 | NMe₂ | H | 564.1 | 565 |
| 8-5 | NHMe | H | 550.1 | 551 |

### Example 9

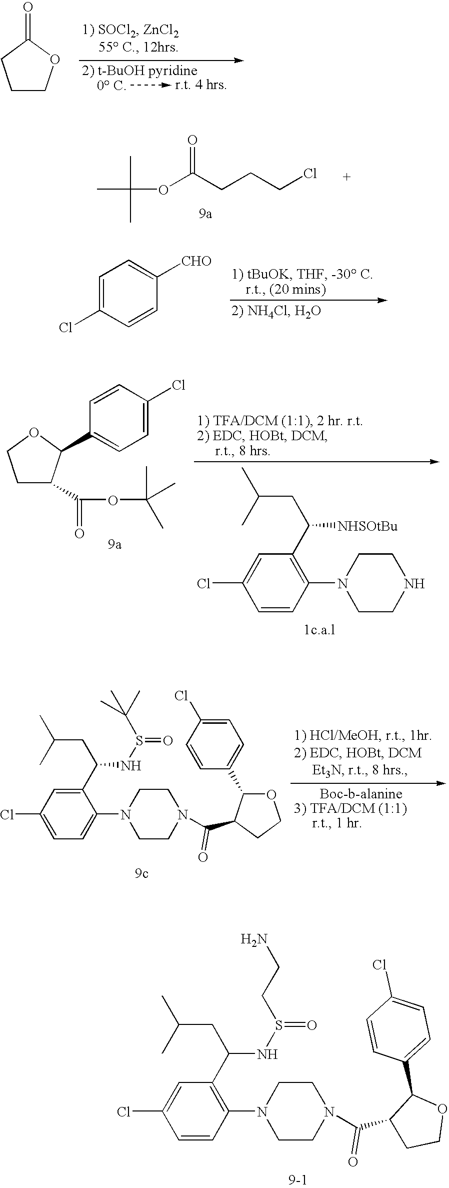

### Step 9A: 4-Chlorobutanoyl Ester 9a

γ-Butyrolactone (7.7 mL, 0.1 mol) was added in one portion to a stirred solution of thionyl chloride (8 mL, 0.11 mol) and anhydrous zinc chloride (0.6 g, 4.4 mmol). The reaction mixture was heated with stirring at 55° C. for 12 hours then purified by fractional distillation at approximately 15-30 mm Hg. The fraction corresponding to a boiling point range of 110-125° C. was collected which provided the intermediate acid chloride (10.4 g, 74 mmol, 74% yield). This intermediate was then added slowly (over 15 minutes) to a cooled (0° C.) solution of pyridine (6 mL, 74 mmol) and t-butanol (8.75 mL, 92 mmol). After the addition, the reaction was stirred at room temperature for 4 hours then partitioned between water and ether. The water layer was acidified with concentrated sulfuric acid and extracted with ether (3×50 mL). The combined organic layers were then washed with 1N HCl solution (3×100 mL), water (100 mL), and saturated NaCl (100 mL). The organic layer was collected, dried over anhydrous Na₂SO₄, filtered, and solvent was removed in vacuo. Compound 9a was recovered as a clear oil in 25% yield (3.28 g, 18.35 mmol) after purification by column chromatography on silica using 100% dichloromethane as the eluent (R_{f}=0.9). ¹H NMR (CDCl₃) 3.59 (t, 2H, CH₂), 2.41 (t, 2H, CH₂), 2.05 (t, 2H, CH₂), 1.45 (s, 9H, CH₃).

### Step 9B: Tetrahydrofuran Acid 9b

A solution of 4-chlorobutanoyl ester 9a (2.8 g, 15.8 mmol) and 4-chlorobenzaldehyde (4.5 g, 31.7 mmol) in THF (16 mL) was cooled to −30° C. and potassium t-butoxide (3.2 g, 28.5 mmol) was added in 3-4 portions. The mixture was allowed to stir for 20 minutes at −30° C. then 10 minutes at room temperature. The reaction mixture was then quenched with aqueous NH₄Cl solution (50 mL) and extracted with dichloromethane (3×60 mL). The organic layer was collected, dried over anhydrous MgSO₄, filtered, and solvent was removed under vacuum. The intermediate tetrahydrofuran t-butyl ester 9b was recovered in 8% yield (338 mg, 1.2 mmol) after purification by column chromatography on silica using 15% ether/petroleum ether as the eluent (R_{f}=0.4). Analysis of proton NMR and comparison to literature references* confirmed the trans isomer as the isolated product. ¹H NMR (CDCl₃) 7.3 (s, 4H, ArH), 4.95 (d, 1H, CH), 4.12-4.17 (m, 1H, CH₂), 3.97-4.05 (m, 1H, CH₂), 2.76-2.84 (m, 1H, CH), 2.22-2.32 (m, 2H, CH₂), 1.44 (s, 9H, CH₃). ref* Judka, M.; Makosza, M. Chem. Eur. J. 2002, 8, No. 18, p4234-4240.

### Step 9C: Tetrahydrofuran Sulfinamide 9c

Tetrahydrofuran t-butyl ester 9b (382 mg, 1.35 mmol) was dissolved in 1:1 TFA/DCM (4 mL) and stirred at room temperature for 2 hours. Solvent and excess TFA was removed in vacuo to give the desired tetrahydrofuran acid in quantitative yield. An aliquot of acid was used for the next step without further purification. The crude tetrahydrofuran acid intermediate (102 mg, 0.45 mmol) was dissolved in DCM (2.25 mL) along with HOBt (61 mg, 0.45 mmol), 2-[1-piperazinyl]-1-[1S-(S-t-butanesulfinamido)-3-methylbutyl]-5-chlorobenzene 1c.a.1 (174 mg, 0.45 mmol, made by the deprotection of 1c.a with TFA/dichloromethane according to Step 1D), and triethylamine (63 μL, 0.45 mmol). The reaction mixture was allowed to stir at room temperature for 10 minutes then EDC (86 mg, 0.45 mmol) was added. The reaction was then allowed to stir at room temperature for an additional 8 hours. After 8 hours, the reaction mixture was washed with saturated NaHCO₃ (3×5 mL) and saturated NaCl (5 mL). The organic layer was collected, dried over anhydrous MgSO₄, filtered, and evaporated to dryness under vacuum. Compound 9c was recovered in 86% yield (232 mg, 0.39 mmol) after purification by column chromatography on silica using 65% ethyl acetate/hexanes as the eluent (R_{f}=0.3). MS: calc. for C₃₀H₄₁Cl₂N₃O₃S: 593.2; Found: 594 (MH⁺); retention time: 2.942 minutes; Method info: APCI positive ion scan 100-1000 Frag V=80; 95% 0.05% TFA/H₂O to 95% ACN/0.05% TFA over 2 min, 3.4 min run, ODS-AQ column.

### Step 9D: 4-Chlorophenyl Tetrahydrofuran 9-1

Tetrahydrofuran sulfinamide 9c (231 mg, 0.39 mmol) was dissolved in MeOH (3.9 mL) and HCl (2M in ether, 254 μL, 0.51 mmol) was added to the reaction vial. The reaction mixture was allowed to stir at room temperature for 1 hour (monitored by TLC). Nitrogen gas was then bubbled through the reaction mixture to evaporate residual HCl then the remaining solvent was removed in vacuo. The residue was dissolved in dichloromethane (10 mL), washed with saturated NaHCO₃ (3×20 mL) and saturated NaCl (20 mL). The organic layer was collected, dried over anhydrous MgSO₄, filtered, and evaporated to dryness under vacuum. A portion of the deprotected intermediate (49 mg, 0.1 mmol) was then dissolved in dichloromethane (0.5 mL) along with HOBt (13.5 mg, 0.1 mmol), Boc-p-alanine (18.9 mg, 0.1 mmol), and triethylamine (14 uL, 0.1 mmol). The reaction mixture was allowed to stir at room temperature for 10 minutes then EDC (19.2 mg, 0.1 mmol) was added. The reaction was then allowed to stir at room temperature for an additional 8 hours. After 8 hours, the reaction mixture was diluted with dichloromethane (3 mL) and washed with saturated NaHCO₃ (2×5 mL). The organic layer was collected and evaporated to dryness under vacuum. The residue was dissolved in 1:1 TFA/DCM (1 mL) and stirred at room temperature for 1 hour. The reaction mixture was then evaporated to dryness under a stream on nitrogen and purified by preparative HPLC. Compound 9-1 was recovered as the TFA salt in 55% yield. MS: calc. for C₂₉H₃₈Cl₂N₄O₃: 560.2; Found: 561 (MH+); retention time: 6.42 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 9 were prepared.

TABLE 9

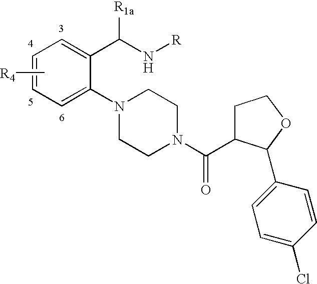

| Cpd | R₄ | R₁ₐ | R | MW | (MH⁺) |
|---|---|---|---|---|---|
| 9-1 | 4-Cl | i-Bu | C(O)CH₂CH₂NH₂ | 560.6 | 561 |
| 9-2 | 4-Cl | i-Bu | C(O)CH₂CH₂NMe₂ | 588.6 | 589 |
| 9-3 | 4-Cl | i-Bu | C(O)CH₂NHMe | 560.6 | 561 |
| 9-4 | 4-CF₃ | i-Bu | C(O)CH₂CH₂NH₂ | 594.1 | 595 |
| 9-5 | 6-F | i-Pr | C(O)CH₂NMe₂ | 530.1 | 531 |
| 9-6 | 6-F | i-Pr | C(O)CH₂CH₂NMe₂ | 558.1 | 559 |
| 9-7 | 4-Cl | i-Bu | H | 489.5 | 490 |
| 9-8 | 4-CF₃ | i-Bu | H | 523 | 524 |
| 9-9 | 6-F | i-Pr | H | 459 | 460 |

### Example 10

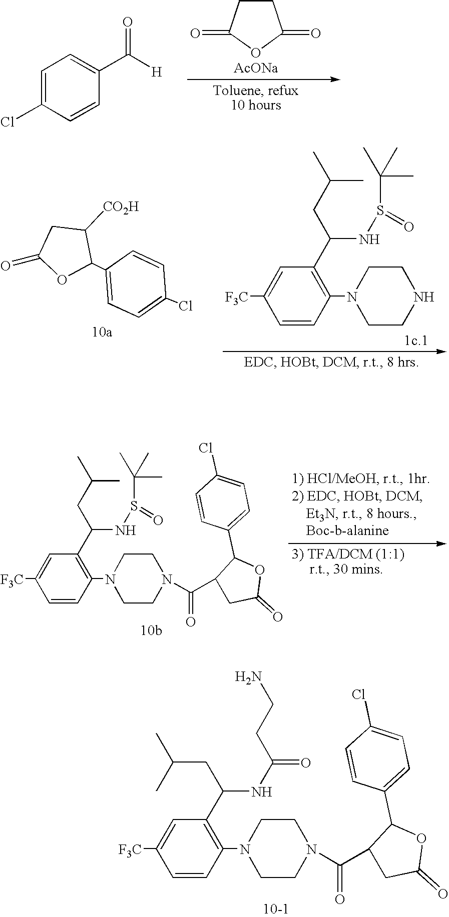

### Step 10A: 4-Chlorophenyl Acid 10a

4-Chlorobenzaldehyde (5 g, 35.6 mmol) was dissolved intoluene (5 mL) along with succinic anhydride (0.71 g, 7.1 mmol) and sodium acetate (1.75 g. 21.3 mmol). The reaction mixture was allowed to reflux for 10 hours under nitrogen atmosphere with constant stirring. After cooling to room temperature, the reaction mixture was diluted with toluene (30 mL) and saturated sodium carbonate solution was added (adjusted to pH ═9). The layers were separated and the water layer was adjusted to pH=2 by addition on concentrated sulfuric acid. The acidic water layer was then extracted with ethyl acetate (3×40 mL). The organic layer was dried over anhydrous Na₂SO₄, filtered, and solvent was removed in vacuo. The crude material was then recrystallized from ethyl acetate/hexanes to give the desired 4-chlorophenylacid 10a in 33% yield (0.57 g, 2.36 mmol). ¹H NMR (CDCl₃) 7.91 (d, 2H, ArH), 7.56 (d, 2H, ArH), 5.57 (d, 1H, CH), 3.40-3.46 (m, H, CH), 2.87-2.91 (m, 2H, CH₂).

### Step 10B: Lactone Sulfinamide 10b

The 4-chlorophenyl acid 10a (71 mg, 0.29 mmol) was dissolved in DCM (1.5 mL) along with HOBt (39 mg, 0.29 mmol), and 2-[1-piperazinyl]-1-[1S-(S-t-butanesulfinamido)-3-methylbutyl]-5-trifluoromethylbenzene 1c.1 (123 mg, 0.29 mmol). The reaction mixture was allowed to stir at room temperature for 10 minutes then EDC (56 mg, 0.29 mmol) was added. The reaction was then allowed to stir at room temperature for an additional 8 hours. After 8 hours, the reaction mixture was washed with saturated NaHCO₃ (3×5 mL) and saturated NaCl (5 mL). The organic layer was collected, dried over anhydrous MgSO₄, filtered, and evaporated to dryness under vacuum. Compound 10b was recovered in 43% yield (80.5 mg, 0.125 mmol) after purification by column chromatography on silica using 60% ethyl acetate/hexanes as the eluent (R_{f}=0.3). MS: calc. for C₃₁H₃₉ClF₃N₃O₄S: 641.2; Found: 642 (MH⁺); retention time: 2.894 minutes; Method info: APCI positive ion scan 100-1000 Frag V=80; 95% 0.05% TFA/H₂O to 95% ACN/0.05% TFA over 2 min, 3.4 min run, ODS-AQ column.

### Step 10C: 4-Chlorophenyl Lactone 10-1

Lactone sulfinamide 10b (81 mg, 0.13 mmol) was dissolved in MeOH (1.25 mL) and HCl (2M in ether, 81.3 uL, 0.16 mmol) was added to the reaction vial. The reaction mixture was allowed to stir until all of the starting material had been consumed (monitored by TLC). Nitrogen gas was then bubbled through the reaction mixture to evaporate residual HCl then the remaining solvent was removed in vacuo. The residue was dissolved in dichloromethane (5 mL), washed with saturated NaHCO₃ (3×5 mL), and saturated NaCl (5 mL). The organic layer was collected, dried over anhydrous MgSO₄, filtered, and evaporated to dryness under vacuum. The crude deprotected amine was recovered in 63% yield and used for the next step without further purification. The deprotected intermediate (43 mg, 0.08 mmol) was then dissolved in dichloromethane (1 mL) along with HOBt (10 mg, 0.08 mmol), and Boc-β-alanine (15 mg, 0.08 mmol). The reaction mixture was allowed to stir at room temperature for 10 minutes then EDC (15 mg, 0.08 mmol) was added. The reaction was then allowed to stir at room temperature for an additional 8 hours. After 8 hours, the reaction mixture was diluted with dichloromethare (3 mL) and washed with saturated NaHCO₃ (2×5 mL). The organic layer was collected and evaporated to dryness under vacuum. The residue was dissolved in 1:1 TFA/DCM (1 mL) and stirred at room temperature for 30 minutes. The reaction mixture was then evaporated to dryness under a stream on nitrogen and purified by preparative HPLC. Compound 10-1 was recovered as the TFA salt in 35% yield. MS: calc. for C₃₀H₃₆ClF₃N₄O₄: 608.2; Found: 609 (MH⁺); retention time: 5.88 minutes; Method info: APCI positive ion scan 100-1000 Frag V=80; 95% 0.025% TFA/H₂O to 95% ACN/0.025% TFA over 13 min, 15.5 min run, ODS-AQ column.

### Example 11

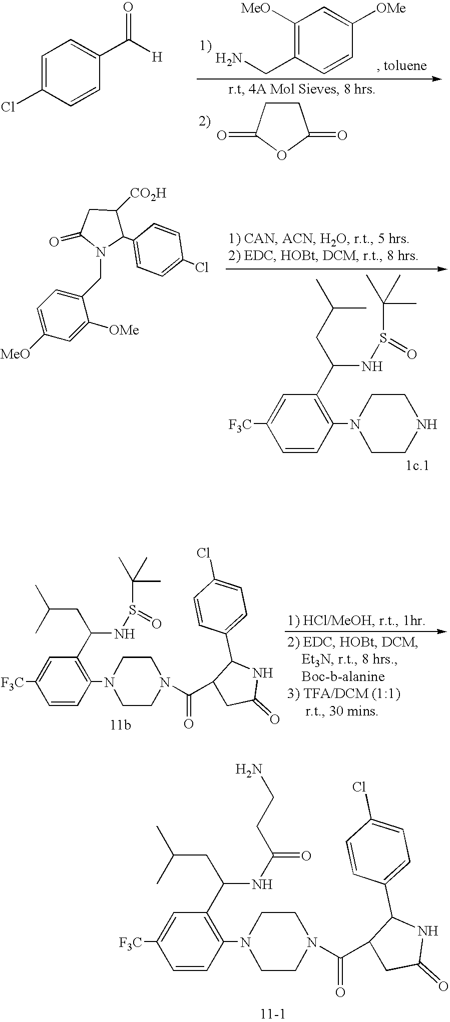

### Step 11A: PMB-Protected Lactam 11a

4-Chlorobenzaldehyde (10 g, 71 mmol) was dissolved intoluene (36 mL) along with 2,4-dimethoxybenzylamine (12.1 mL, 80.4 mmol) and 4A molecular sieves (14.5 g). The reaction mixture was allowed to stir at room temperature for 8 hours under nitrogen atmosphere then solvent was removed in vacuo. The crude imine intermediate was used for the next step without any further purification. The crude imine (20 g, 71 mmol) was dissolved in o-xylene (72 mL) along with succinic anhydride (7.1 g, 71 mmol) and refluxed under nitrogen atmosphere for 4 hours. After cooling to room temperature, the solid was filtered off and then dissolved in 7:10 methanol/dichloromethane (100 mL). The solution was treated with decolorizing carbon, filtered through Celite®, and the solution was concentrated to about 40 mL. The resulting solid was filtered off and washed with 1:2 methylene chloride/ether mixture to give the compound 11a in 56% yield (15.6 g, 40.1 mmol). The material was used in the next step without any further purification.

### Step 11B: Lactam Sulfinamide 11b

A solution of PMB-protected lactam 11a (1 g, 2.6 mmol) in acetonitrile (25 mL) was treated with a solution of ceric ammonium nitrate (4.2 g, 7.7 mmol) in water (38 mL) over 5 minutes. The reaction was allowed to stir at room temperature under nitrogen atmosphere for 5 hours. The reaction mixture was extracted with ethyl acetate (3×50 mL) and the organic phases were washed with 5% sodium bicarbonate (100 mL). The aqueous layer was backwashed with ethyl acetate (100 mL) and combined with the organic extracts. The organic layer was then washed with 10% sodium sulfate (150 mL), 5% sodium bicarbonate (150 mL), and saturated NaCl solution (150 mL). The organic solution was treated with decolorizing carbon, filtered through Celite®, and evaporated to dryness in vacuo to give the crude deprotected lactam intermediate in 67% yield (0.41 g, 1.7 mmol). This material was used for the next step without further purification. The crude deprotected lactam intermediate (240 mg, 1 mmol) was then dissolved in dichloromethane (5 mL) along with HOBt (135 mg, 1 mmol), and 2-[1-piperazinyl]-1-[1S-(S-t-butanesulfinamido)-3-methylbutyl]-5-trifluoromethylbenze 1c.1 (420 mg, 1 mmol). The reaction mixture was allowed to stir at room temperature for 10 minutes then EDC (192 mg, 1 mmol) was added. The reaction was then allowed to stir at room temperature for an additional 8 hours. After 8 hours, the reaction mixture was diluted with dichloromethane (5 mL), washed with saturated NaHCO₃ (2×10 mL), and saturated NaCl solution (30 mL). The organic layer was collected and evaporated to dryness under vacuum. Compound 11b was recovered in 34% yield (220 mg, 0.34 mmol) after purification by column chromatography on silica using 10% methanol/methylene chloride as the eluent (R_{f}=0.4). MS: calc. for C₃₁H₄₀ClF₃N₄O₃S: 640.25; Found: 641 (MH⁺); retention time: 2.747 minutes; Method info: APCI positive ion scan 100-1000 Frag V=80; 95% 0.05% TFA/H₂O to 95% ACN/0.05% TFA over 2 min, 3.4 min run, ODS-AQ column.

### Step 11C: 4-Chlorophenyl Lactam 11-1

Lactam sulfinamide 11b (220 mg, 0.34 mmol) was dissolved in MeOH (3.4 mL) and HCl (2M in ether, 222 μL, 0.44 mmol) was added to the reaction vial. The reaction mixture was allowed to stir at room temperature for 1 hour (monitored by TLC). Nitrogen gas was then bubbled through the reaction mixture to evaporate residual HCl then the remaining solvent was removed in vacuo. The residue was dissolved in dichloromethane (5 mL), washed with saturated NaHCO₃ (3×8 mL) and saturated NaCl (8 mL). The organic layer was collected, dried over anhydrous MgSO₄, filtered, and evaporated to dryness under vacuum. The crude deprotected amine was recovered in 98% yield and an aliquot was used for the next step without further purification. The deprotected intermediate (54 mg, 0.1 mmol) was then dissolved in dichloromethane (0.5 mL) along with HOBt (13.5 mg, 0.1 mmol), and Boc-β-alanine (18.9 mg, 0.1 mmol). The reaction mixture was allowed to stir at room temperature for 10 minutes then EDC (19 mg, 0.1 mmol) was added. The reaction was then allowed to stir at room temperature for an additional 8 hours. After 8 hours, the reaction mixture was diluted with dichloromethane (3 mL) and washed with saturated NaHCO₃ (2×5 mL). The organic layer was collected and evaporated to dryness under vacuum. The residue was dissolved in 1:1 TFA/DCM (1 mL) and stirred at room temperature for 30 minutes. The reaction mixture was then evaporated to dryness under a stream on nitrogen and purified by preparative HPLC. Compound 11-1 was recovered as the TFA salt in 12% yield. MS: calc. for C₃₀H₃₇ClF₃N₅O₃: 607.2; Found: 608 (MH⁺); retention time: 5.55 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 11 were prepared.

TABLE 11

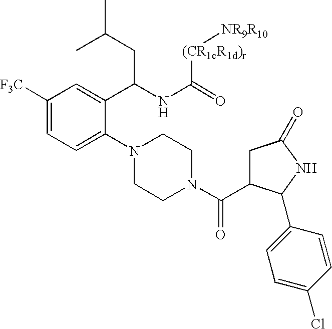

| Cpd | (CR_{1c}R_{1d})ᵣNR₉R₁₀ | MW | (MH⁺) |
|---|---|---|---|
| 11-1 | CH₂CH₂NH₂ | 607.1 | 608 |
| 11-2 | CH₂CH₂NMe₂ | 635.1 | 636 |
| 11-3 | CH₂NHMe | 607.1 | 608 |

### Example 12

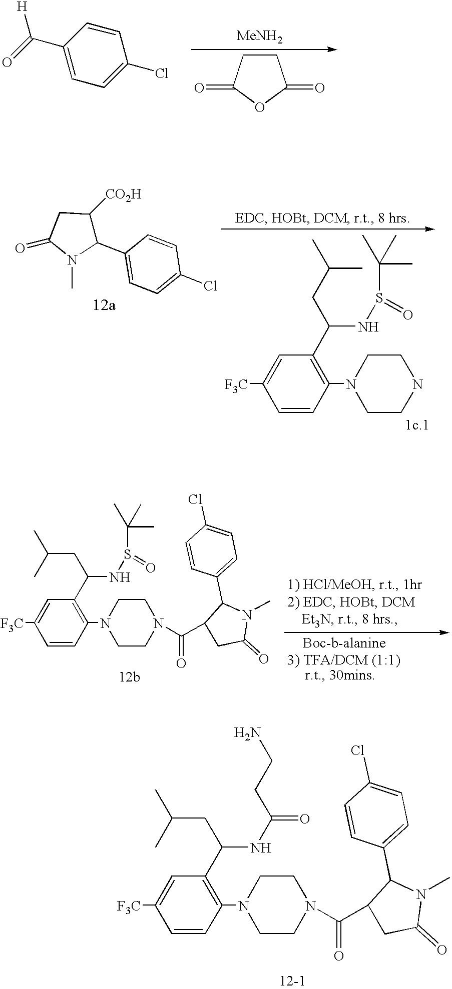

### Step 12A: 4-Chlorophenyl Lactam 12a

4-Chlorobenzaldehyde (3 g, 21 mmol) was dissolved in toluene (30 mL) along with methylamine (32 mL, 2M in THF, 64 mmol) and 4A molecular sieves (14.5 g). The reaction mixture was allowed to stir at room temperature for 8 hours under nitrogen atmosphere then solvent was removed in vacuo. The crude imine intermediate was used for the next step without any further purification. The crude imine (3.3 g, 21.34 mmol) was dissolved in o-xylene (22 mL) along with succinic anhydride (2.1 g, 21 mmol) and refluxed under nitrogen atmosphere for 4 hours. After cooling to room temperature, the solid was filtered off and then dissolved in 7:10 methanol/dichloromethane (50 mL). The solution was treated with decolorizing carbon, filtered through Celite®, and solution was concentrated to about 20 mL. The resulting solid was filtered off and washed with 1:2 methylene chloride/ether mixture to give the crude product which was recrystallized from ethyl acetate/hexanes to provide the 4-chlorophenyl lactam 12a in 41% yield (2.2 g, 8.8 mmol). MS: calc. for C₁₂H₁₂ClNO₃: 253.1; Found: GC-MS m/z 253 (MH⁺).

### Step 12B: Lactam Sulfinamide 12b

4-Chlorophenyl lactam 12a (761 mg, 3 mmol) was dissolved in dichloromethane (15 mL) along with HOBt (405 mg, 3 mmol) and 2-[I -piperazinyl]-1-[1S-(S-t-butanesulfinamido)-3-methylbutyl]-5-trifluoromethylbenzene 1c.1 (1.3 g, 3 mmol). The reaction mixture was allowed to stir at room temperature for 10 minutes then EDC (575 mg, 3 mmol) was added. The reaction was then allowed to stir at room temperature for an additional 8 hours. After 8 hours, the reaction mixture was diluted with dichloromethane (10 mL), washed with saturated NaHCO₃ (2×30 mL), and saturated NaCl solution (30 mL). The organic layer was collected and evaporated to dryness under vacuum. Compound 12b was recovered in 49% yield (0.97 g, 1.47 mmol) after purification by column chromatography on silica using 90% methanol/methylene chloride as the eluent (R_{f}=0.3). MS: calc. for C₃₂H₄₂ClF₃N₄O₃S: 654.3; Found: 655 (MH+); retention time: 3.06 minutes; Method info: APCI positive ion scan 100-1000 Frag V=80; 95% 0.05% TFA/H₂O to 95% ACN/0.05% TFA over 2 min, 3.4 min run, ODS-AQ column.

### Step 12C: 4-Chlorophenyl Lactam 12-1

Lactam sulfinamide 12b (0.96 g, 1.5 mmol) was dissolved in MeOH (14.6 mL) and HCl (2M in ether, 952 μL, 1.9 mmol) was added to the reaction vial. The reaction mixture was allowed to stir at room temperature for 1 hour. Nitrogen gas was then bubbled through the reaction mixture to evaporate residual HCl and the remaining solvent was removed in vacuo. The residue was dissolved in dichloromethane (10 mL), washed with saturated NaHCO₃ (3×20 mL) and saturated NaCl (20 mL). The organic layer was collected, dried over anhydrous MgSO₄, filtered, and evaporated to dryness under vacuum. An aliquot of the crude deprotected amine was used for the next step without further purification. The deprotected intermediate (55 mg, 0.1 mmol) was then dissolved in dichloromethane (0.5 mL) along with HOBt (13.5 mg, 0.1 mmol), and Boc-p-alanine (18.9 mg, 0.1 mmol). The reaction mixture was allowed to stir at room temperature for 10 minutes then EDC (19 mg, 0.1 mmol) was added. The reaction was then allowed to stir at room temperature for an additional 8 hours. After 8 hours, the reaction mixture was diluted with dichloromethane (3 mL) and washed with saturated NaHCO₃ (2×5 mL). The organic layer was collected and evaporated to dryness under vacuum. The residue was dissolved in 1:1 TFA/DCM (1 mL) and stirred at room temperature for 30 minutes. The reaction mixture was then evaporated to dryness under a stream on nitrogen and purified by preparative HPLC. Compound 12-1 was recovered as the TFA salt in 49% yield. MS: calc. for C₃₁H₃₉ClF₃N₅O₃: 621.3; Found: 622 (MH⁺); retention time: 6.52 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 12 were prepared.

TABLE 12

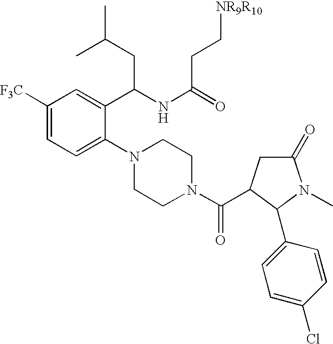

| Cpd | NR₉R₁₀ | MW | (MH⁺) |
|---|---|---|---|
| 12-1 | NH₂ | 621.1 | 622 |
| 12-2 | NMe₂ | 649.2 | 650 |
| 12-3 | 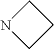 | 661.2 | 662 |

### Example 13

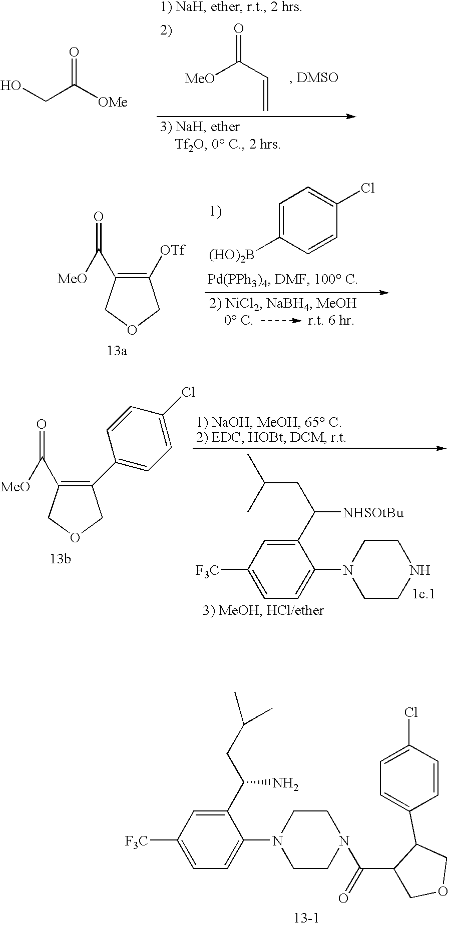

### Step 13A: 2,5-Dihydofuran Ester 13a

Sodium hydride (4 g, 60% w/w in oil dispersion, 100 mmol) was added to a flame-dried flask along with ether (100 mL). To the reaction flask under nitrogen atmosphere, methyl glycolate (7.7 mL, 100 mmol) was added slowly with constant stirring. The reaction mixture was allowed to stir at room temperature for 2 hours under nitrogen atmosphere then solvent was removed in vacuo. To the residue, methyl acrylate (10.8 mL, 120 mmol) in DMSO (50 mL) was added in one portion while the reaction flask was kept immersed in an ice bath. The reaction mixture was allowed to stir at 0° C. for 15 minutes then at room temperature for 1 hour. The reaction mixture was then filtered through Celite®, poured into ice-cold aqueous sulfuric acid solution (150 mL, 2N), and extracted with ether (2×200 mL). The organic layer was washed with saturated NaCl solution (500 mL), dried over anhydrous Na₂SO₄, filtered, and solvent was removed in vacuo. The intermediate ketoester was recovered in 26% yield (3.7 g, 25.7 mmol) after purification by column chromatography on silica using 25% ethyl acetate/hexanes as the eluent (R_{f}=0.3). The ketoester intermediate (3.7 g, 25.7 mmol) was added slowly to a solution of sodium hydride (1.4 g, 60% w/w in oil dispersion, 34 mmol) in ether (80 mL) at 0° C. with constant stirring under nitrogen atmosphere. After 30 minutes, trifluoromethanesulfonic anhydride (5.3 mL, 31.4 mmol) was added dropwise over 5 minutes. The reaction mixture was allowed to stir at 0° C. for an additional 1.5 hours then the reaction was poured into water (80 mL) and the layers were separated. The aqueous phase was washed with dichloromethane (2×60 mL) and the organic phases were combined. The organic layer was dried over anhydrous Na₂SO₄, filtered, and solvent was removed in vacuo. The 2,5-dihydrofuran ester 13a was recovered in 23% yield (1.6 g, 5.8 mmol) after purification by column chromatography on silica using 25% ethyl acetate/hexanes as the eluent (R_{f}=0.45). MS: calc. for C₇H₇F₃O₆S: 257.9; Found: GC-MS m/z 275 (MH⁺).

### Step 13B: 4-Chlorophenyltetrahydrofuran 13b

To an oven-dried flask, 2,5-dihydofuran ester 13a (1.2 g, 4.3 mmol) was dissolved in DMF (24 mL) along with 4-chlorophenylboronic acid (0.9 g, 5.6 mmol), triethylamine (1.82 mL, 12.9 mmol), and palladium (0) tetrakistriphenylphosphine (0.15 g, 0.1 mmol). The reaction mixture was stirred under nitrogen atmosphere at 100° C. for 12 hours. After cooling to room temperature, the mixture was partitioned between ethyl acetate (50 mL) and water (50 mL). The organic layer was washed with water (2×50 mL), saturated potassium carbonate (50 mL), and saturated NaCl (50 mL). The organic layer was dried over anhydrous Na₂SO₄, filtered, and solvent was removed in vacuo. The intermediate 4-chlorophenyl-2,5-dihydofuran ester was recovered in 40% yield (0.42 g, 1.76 mmol) after purification by column chromatography on silica using 100% dichloromethane as the eluent (R_{f}=0.6). A portion of this 4-chlorophenyl-2,5-dihydofuran intermediate (0.36 g, 1.5 mmol) was dissolved in methanol (7 mL) along with nickel (II) chloride (0.02 g, 0.15 mmol) and the reaction mixture was cooled to 0° C. To the cold reaction mixture, sodium borohydride (0.11 g, 2.9 mmol) was added in small portions (the reaction turned black during this time due to formation of nickel boride) then the reaction was allowed to stir at room temperature for 6 hours. The reaction was then filtered and the black solid was washed with methanol. The organic phases were combined and solvent was removed in vacuo. The residue was dissolved in ethyl acetate (10 mL) and washed with water (2×10 mL). The organic layer was further washed with IN HCl solution (2×10 mL), saturated NaCl solution (30 mL), dried over anhydrous Na₂SO₄, filtered, and solvent was removed in vacuo. Compound 13b was recovered in 76% yield (0.32 g, 1.34 mmol) and used for the next step without further purification. MS: calc. for C₁₂H₁₃ClO₃: 240.1; Found: GC-MS m/z 238 (MH⁺).

### Step 13C: Amino-4-chlorophenyltetrahydrofuran 13-1

4-Chlorophenyltetrahydrofuran 13b (0.32 g, 1.34 mmol) was dissolved in methanol (12 mL) and sodium hydroxide solution in water (2.5 mL, 2.5N, 6.25 mmol) was added. The reaction mixture was allowed to stir at 65° C. for 3 hours then methanol was removed in vacuo. The aqueous layer was acidified with concentrated HCl solution and extracted with ethyl acetate. The organic phases were dried over anhydrous Na₂SO₄, filtered, and solvent was removed in vacuo. Compound 13b.1 was recovered in 97% yield (0.29 g, 1.3 mmol) and used for the next step without further purification. An aliquot of the crude tetrahydrofuran acid intermediate 13b.1 (22 mg, 0.1 mmol) was then dissolved in dichloromethane (0.5 mL) along with HOBt (13.5 mg, 0.1 mmol), and 2-[1-piperazinyl]-1-[1S-(S-t-butanesulfinamido)-3-methylbutyl]-5-trifluoromethylbenzene 1c.1 (42 mg, 0.1 mmol). The reaction mixture was allowed to stir at room temperature for 10 minutes then EDC (19 mg, 0.1 mmol) was added. The reaction was then allowed to stir at room temperature for an additional 8 hours. After 8 hours, the reaction mixture was diluted with dichloromethane (3 mL) and washed with saturated NaHCO₃ (2×5 mL). The organic layer was collected and evaporated to dryness under vacuum. The residue was dissolved in MeOH (1 mL) and HCl (2M in ether, 65 uL, 0.13 mmol) was added to the reaction vial. The reaction mixture was allowed to stir at room temperature until all of the starting material had been consumed (monitored by TLC). Nitrogen gas was then bubbled through the reaction mixture to evaporate residual HCl then the remaining solvent was removed in vacuo. The residue was purified by preparative HPLC to give compound 13-1 as the TFA salt in 21% yield. MS: calc. for C₂₇H₃₃ClF₃N₃O₂: 523.2; Found: 524 (MH⁺); retention time: 6.45 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 13 were prepared.

TABLE 13

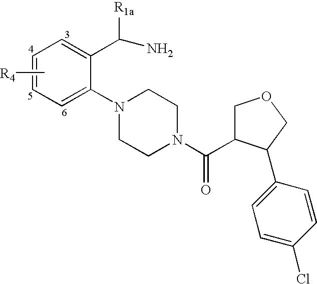

| Cpd | R₄ | R₁ₐ | MW | (MH⁺) |
|---|---|---|---|---|
| 13-1 | 4-CF₃ | i-Bu | 523 | 524 |
| 13-2 | 4-Cl | i-Bu | 489.5 | 490 |
| 13-3 | 6-F | i-Pr | 459 | 460 |

### Example 14

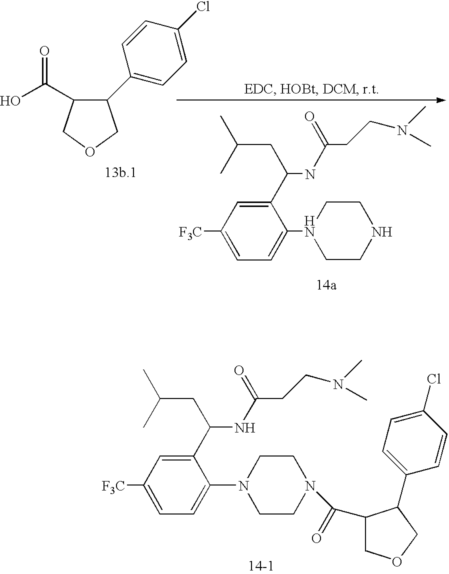

### Step 14A: Amino-4-chlorophenyltetrahydrofuran 14-1

An aliquot of the crude tetrahydrofuran acid intermediate from above 13b.1 (22 mg, 0.1 mmol) was dissolved in dichloromethane (0.5 mL) along with HOBt (13.5 mg, 0.1 mmol), and trifluoromethylphenyl piperazine 14a (42 mg, 0.1 mmol, made from compound 1c by deprotecting the sulfinamide and reaction with 3-dimethylaminopropionic acid according to Step 7C followed by deprotection of the BOC with TFA/dichloromethane as in Step 7B). The reaction mixture was allowed to stir at room temperature for 10 minutes then EDC (19 mg, 0.1 mmol) was added. The reaction was then allowed to stir at room temperature for an additional 8 hours. After 8 hours, the reaction mixture was diluted with dichloromethane (3 mL) and washed with saturated NaHCO₃ (2×5 mL). The organic layer was collected and evaporated to dryness under vacuum. The residue was purified by preparative HPLC to give compound 14-1 as the TFA salt in 14% yield. MS: calc. for C₃₂H₄₂ClF₃N₄O₃: 622.3; Found: 623 (MH⁺); retention time: 6.91 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 14 were prepared.

TABLE 14

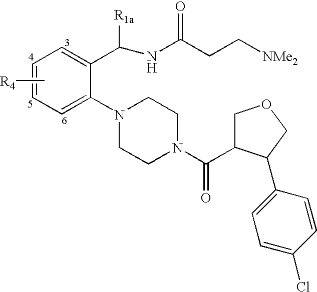

| Cpd | R₄ | R₁ₐ | MW | (MH⁺) |
|---|---|---|---|---|
| 14-1 | 4-CF₃ | i-Bu | 622.2 | 623 |
| 14-2 | 4-Cl | i-Bu | 588.6 | 589 |
| 14-3 | 6-F | i-Pr | 558.1 | 559 |

### Example 15

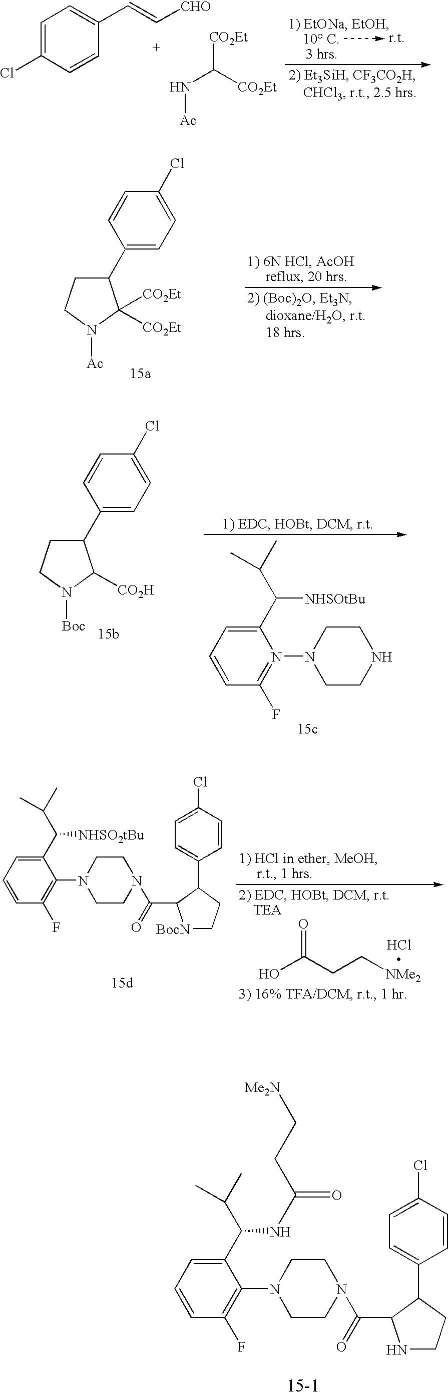

### Step 15A: 4-Chlorophenyl pyrrolidine 15a

A solution of 3-(4-chlorophenyl)-propenal (1.5 g, 9 mmol) in ethanol (4 mL) was added slowly to a mixture of diethyl acetamidomalonate (1.9 g, 8.8 mmol) and sodium ethoxide (0.6 g, 8.82 mmol) in ethanol (5.6 mL) at 10° C. After the addition was complete, the reaction mixture was allowed to stir at room temperature for 3 hours then quenched with glacial acetic acid (0.2 mL). Solvent was then removed under vacuum and the residue was dissolved in dichloromethane (40 mL) then washed with saturated NaHCO₃ (3×50 mL) followed by saturated NaCl solution (50 mL). The organic layer was collected, dried over anhydrous MgSO₄, filtered, and evaporated to dryness under vacuum. The hydroxypyrrolidine intermediate was recovered in 86% yield (2.9 g, 7.6 mmol) after purification by column chromatography on silica using 75% ethyl acetate/hexanes as the eluent (R_{f}=0.3). To a solution of hydroxypyrrolidine intermediate (2.9 g, 7.6 mmol) and triethylsilane (1.8 mL, 11.34 mmol) in chloroform (15 mL) was added trifluoroacetic acid (5.6 mL, 75.6 mmol) dropwise with stirring over 10 minutes. The reaction was allowed to stir at room temperature for 2.5 hours then solvent and TFA was removed in vacuo. The residue was dissolved in ethyl acetate (35 mL) then washed with saturated NaHCO₃ (3×50 mL) followed by saturated NaCl solution (50 mL). The organic layer was collected, dried over anhydrous Na₂SO₄, filtered, and evaporated to dryness under vacuum. The 4-chlorophenyl pyrrolidine 15a was recovered in 85% yield (2.4 g, 6.5 mmol) after purification by column chromatography on silica using 70% ethyl acetate/hexanes as the eluent (R_{f}=0.3). MS: calc. for C₁₈H₂₂CINO₅: 367.1; Found: 368 (MH⁺); retention time: 2.67 minutes; Method info: APCI positive ion scan 100-1000 Frag V=80; 95% 0.05% TFA/H₂O to 95% ACN/0.05% TFA over 2 min, 3.4 min run, ODS-AQ column.

### Step 15B: Boc-Pyrrolidine Acid 15b

4-Chlorophenyl pyrrolidine 15a (2.4 g, 6.5 mmol) was refluxed in 6N HCl (11.2 mL) along with glacial acetic acid (2.8 mL) for 20 hours. The reaction was then extracted with ethyl acetate (2×15 mL). The aqueous phase was concentrated in vacuo then triturated with ether to crystallize the product. This product was combined with the ethyl acetate extracts, dried over anhydrous MgSO₄, filtered, and solvent removed in vacuo. The crude material was recrystallized from ethyl acetate/hexanes to give the amino acid hydrochloride salt (1.3 g, 4.95 mmol) in 76% yield. This solid was dissolved in 1:1 dioxane/H₂O (25 mL) along with triethylamine (3.1 mL, 22 mmol) and Boc-anhydride (2.4 g, 10.9 mmol) was added in small portions with constant stirring. The reaction was allowed to stir at room temperature for 18 hours. Solvent was then removed under vacuum and the residue was dissolved in ethyl acetate. The organic phase was washed with 1N HCl, dried over anhydrous Na₂SO₄, filtered, and solvent was removed in vacuo. The crude material was recrystallized from ethyl acetate/hexanes to give the Boc-pyrrolidine acid 15b (1.6 g, 4.95 mmol) in 100% yield from the amino acid intermediate.

### Step 15C: Boc-Pyrrolidine Sulfinamide 15d

Boc-pyrrolidine acid 15b (651.6 mg, 2 mmol) was dissolved in dichloromethane (10 mL) along with HOBt (270 mg, 2 mmol), and fluorophenyl piperazine 15c (711 mg, 2 mmol, made from the BOC deprotection of compound 1c.d with TFA/methylene chloride as in Step 7B). The reaction mixture was allowed to stir at room temperature for 10 minutes then EDC (383 mg, 2 mmol) was added. The reaction was then allowed to stir at room temperature for an additional 8 hours. After 8 hours, the reaction mixture was diluted with dichloromethane (10 mL), washed with saturated NaHCO₃ (2×30 mL), and saturated NaCl solution (30 mL). The organic layer was collected and evaporated to dryness under vacuum. Compound 15d was recovered in 59% yield (0.8 g, 1.2 mmol) after purification by column chromatography on silica using 75% ethyl acetate/hexanes as the eluent (R_{f}=0.3). MS: calc. for C₃₄H₄₈ClFN₄O₄S: 662.3; Found: 663 (MH⁺); retention time: 2.935 minutes; Method info: APCI positive ion scan 100-1000 Frag V=80; 100% 0.05% TFA/H₂O to 90% ACN/0.05% TFA over 2 min, 2.5 min run, ODS-AQ column.

### Step 15D: 2-Fluorophenyl Pyrrolidine 15-1

Boc-pyrrolidine sulfinamide 15d (0.8 g, 1.2 mmol) was dissolved in MeOH (15.5 mL) and HCl (2M in ether, 774 μL, 1.55 mmol) was added to the reaction vial. The reaction mixture was allowed to stir at room temperature until all of the starting material had been consumed (monitored by TLC). Nitrogen gas was then bubbled through the reaction mixture to evaporate residual HCl then the remaining solvent was removed in vacuo. The residue was dissolved in dichloromethane (10 mL), washed with saturated NaHCO₃ (3×40 mL) and saturated NaCl (40 mL). The organic layer was collected, dried over anhydrous MgSO₄, filtered, and evaporated to dryness under vacuum. An aliquot of the crude deprotected amine was used for the next step without further purification. The deprotected amino intermediate (560 mg, 1 mmol) was then dissolved in dichloromethane (5 mL) along with HOBt (135 mg, 1 mmol), 3-dimethylaminopropionic acid hydrochloride (154 mg, 1 mmol), and triethylamine (420 μL, 1.5 mmol). The reaction mixture was allowed to stir at room temperature for 10 minutes then EDC (192 mg, 1 mmol) was added. The reaction was then allowed to stir at room temperature for an additional 8 hours. After 8 hours, the reaction mixture was diluted with dichloromethane (5 mL) and washed with saturated NaHCO₃ (2×15 mL). The organic layer was collected and evaporated to dryness under vacuum. The residue was dissolved in 1:1 TFA/DCM (5 mL) and stirred at room temperature for 30 minutes. The reaction mixture was then evaporated to dryness under a stream on nitrogen to give the crude 15-1 (0.36 g, 0.64 mmol) in 57% yield over 3 steps. A small portion was purified by preparative HPLC to give compound 15-1 as the TFA salt in 12% yield. MS: calc. for C₃₀H₄₁ClFN₅O₂: 557.3; Found: 558 (MH⁺); retention time: 4.639 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 15 were prepared.

TABLE 15

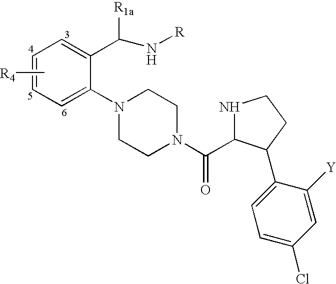

| Cpd | R₄ | R₁ₐ | R | Y | MW | MH⁺ |
|---|---|---|---|---|---|---|
| 15-1 | 6-F | i-Pr | COCH₂CH₂NMe₂ | H | 557.1 | 558 |
| 15-2 | 6-F | i-Pr | COCH₂CH₂NMe₂ | Cl | 591.6 | 592 |
| 15-3 | 4-Cl | i-Bu | COCH₂CH₂NMe₂ | H | 587.6 | 588 |
| 15-4 | 4-Cl | i-Bu | COCH₂CH₂NMe₂ | Cl | 622.1 | 623 |
| 15-5 | 4-CF₃ | i-Bu | COCH₂CH₂NMe₂ | H | 621.2 | 622 |
| 15-6 | 4-CF₃ | i-Bu | COCH₂CH₂NH₂ | H | 593.1 | 594 |
| 15-7 | 6-F | i-Pr | H | H | 458 | 459 |
| 15-8 | 6-F | i-Pr | H | Cl | 492.5 | 493 |
| 15-9 | 4-CF₃ | i-Bu | H | H | 522 | 523 |
| 15-10 | 4-Cl | i-Bu | H | H | 488.5 | 489 |
| 15-11 | 4-Cl | i-Bu | H | Cl | 522.9 | 523 |

### Example 16

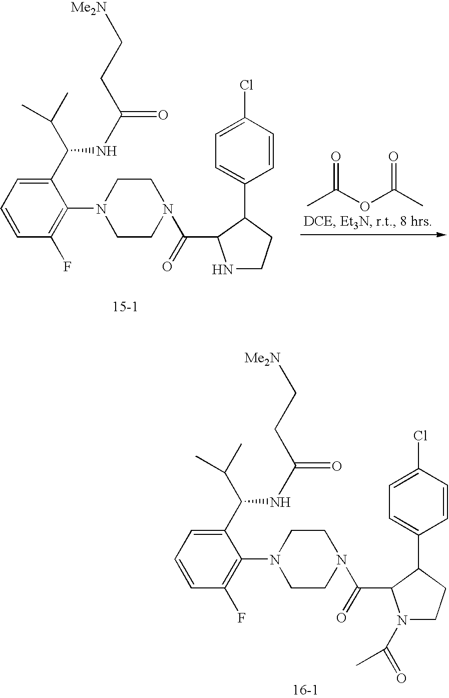

### Step 16A: Dimethylamino Acetyl Pyrrolidine 16-1

2-Fluorophenyl pyrrolidine 15-1 (56 mg, 0.1 mmol) was dissolved in dichloroethane (0.5 mL) along with triethylamine (14 μL, 0.1 mmol) and acetic anhydride (11 μL, 0.1 mmol). The reaction mixture was allowed to stir at room temperature for 8 hours then diluted with dichloromethane (2 mL). The organic layer was washed with saturated NaHCO₃ (3×5 mL), saturated NaCl (5 mL), and solvent was evaporated under a stream of nitrogen. 10 The residue was purified by preparative HPLC to give compound 16-1 as the TFA salt in 15% yield. MS: calc. for C₃₂H₄₃ClFN₅O₃: 599.3; Found: 600 (MH⁺); retention time: 5.513 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 16 were prepared.

TABLE 16

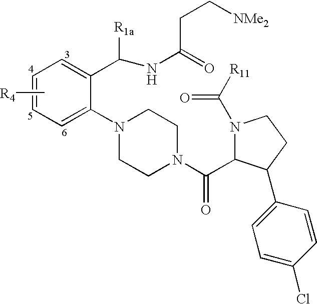

| Cpd | R₄ | R₁ₐ | R₁₁ | MW | (MH⁺) |
|---|---|---|---|---|---|
| 16-1 | 6-F | i-Pr | Me | 599.2 | 600 |
| 16-2 | 6-F | i-Pr | Et | 613.2 | 614 |
| 16-3 | 4-Cl | i-Bu | Me | 629.7 | 630 |
| 16-4 | 4-Cl | i-Bu | Et | 643.7 | 644 |

### Example 17

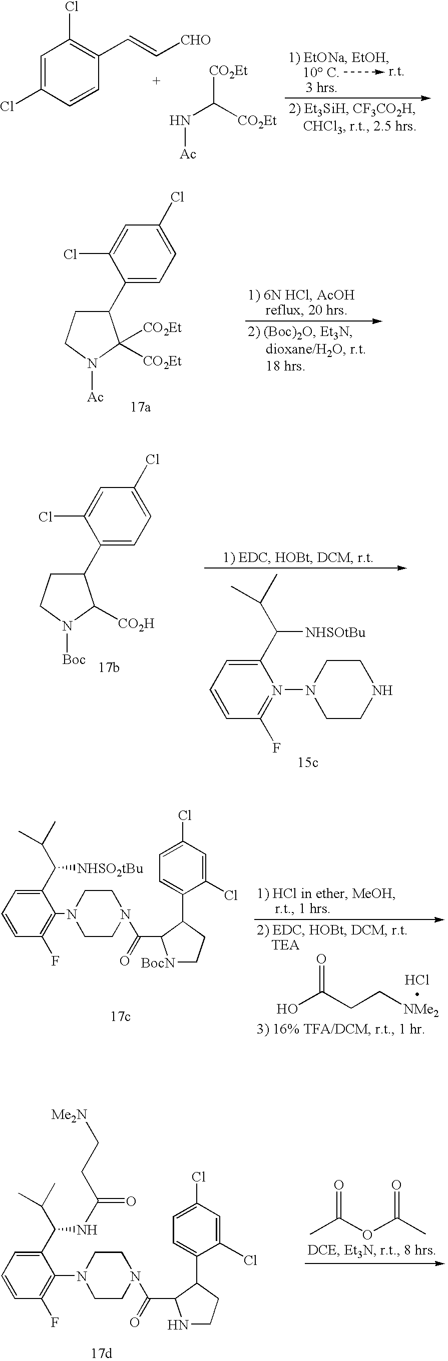

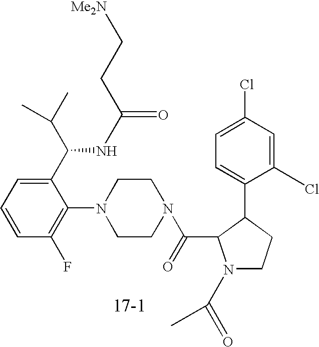

### Step 17A: 2.4-Dichlorophenyl pyrrolidine 17a

A solution of 3-(2,4-chlorophenyl)propenal (1.5 g, 9 mmol) in ethanol (4 mL) was added slowly to a mixture of diethyl acetamidomalonate (1.9 g, 8.8 mmol) and sodium ethoxide (0.6 g, 8.82 mmol) in ethanol (5.6 mL) at 10° C. After the addition was complete, the reaction mixture was allowed to stir at room temperature for 3 hours then quenched with glacial acetic acid (0.2 mL). Solvent was then removed under vacuum and the residue was dissolved in dichloromethane (40 mL) then washed with saturated NaHCO₃ (3×50 mL) followed by saturated NaCl solution (50 mL). The organic layer was collected, dried over anhydrous MgSO₄, filtered, and evaporated to dryness under vacuum. The hydroxypyrrolidine intermediate was recovered in 75% yield (2.8 g, 6.6 mmol) after purification by column chromatography on silica using 75% ethyl acetate/hexanes as the eluent (R_{f}=0.4). To a solution of hydroxypyrrolidine intermediate (2.8 g, 6.6 mmol) and triethylsilane (1.6 mL, 9.9 mmol) in chloroform (13 mL) was added trifluoroacetic acid (4.9 mL, 66 mmol) dropwise with stirring over 10 minutes. The reaction was allowed to stir at room temperature for 2.5 hours then solvent and TFA was removed in vacuo. The residue was dissolved in ethyl acetate (35 mL) then washed with saturated NaHCO₃ (3×50 mL) followed by saturated NaCl solution (50 mL). The organic layer was collected, dried over anhydrous Na₂SO₄, filtered, and evaporated to dryness under vacuum. 2,4-Dichlorophenyl pyrrolidine 17a was recovered in 92% yield (2.4 g, 6.1 mmol) after purification by column chromatography on silica using 70% ethyl acetate/hexanes as the eluent (R_{f}=0.3). MS: calc. for C₁₈H₂₁Cl₂NO₅: 401.1; Found: 402 (MH⁺); retention time: 2.718 minutes; Method info: APCI positive ion scan 100-1000 Frag V=80; 95% 0.05% TFA/H₂O to 95% ACN/0.05% TFA over 2 min, 3.4 min run, ODS-AQ column.

### Step 17B: Boc-Pyrrolidine Acid 17b

2,4-Dichlorophenyl pyrrolidine 17a (2.45 g, 6.1 mmol) was refluxed in 6N HCl (10.5 mL) along with glacial acetic acid (2.6 mL) for 20 hours. The reaction was then extracted with ethyl acetate (2×15 mL). The aqueous phase was concentrated in vacuo then triturated with ether to crystallize the product. This product was combined with the ethyl acetate extracts, dried over anhydrous MgSO₄, filtered, and solvent removed in vacuo. The crude material was recrystallized from ethyl acetate/hexanes to give the amino acid hydrochloride salt (0.85 g, 2.88 mmol) in 47% yield. This solid was dissolved in 1:1 dioxane/H₂O (20 mL) along with triethylamine (1.8 mL, 12.8 mmol) and Boc-anhydride (1.4 g, 6.3 mmol) was added in small portions with constant stirring. The reaction was allowed to stir at room temperature for 18 hours. Solvent was then removed under vacuum and the residue was dissolved in ethyl acetate. The organic phase was washed with 1N HCl, dried over anhydrous Na₂SO₄, filtered, and solvent was removed in vacuo. The crude material was recrystallized from ethyl acetate/hexanes to give the Boc-pyrrolidine acid 17b (0.97 g, 2.7 mmol) in 93% yield from the amino acid intermediate.

### Step 17C: Boc-Pyrrolidine Sulfinamide 17c

Boc-pyrrolidine acid 17b (486 mg, 1.35 mmol) was dissolved in dichloromethane (7 mL) along with HOBt (182 mg, 1.35 mmol), and fluorophenyl piperazine 15c (480 mg, 1.35 mmol). The reaction mixture was allowed to stir at room temperature for 10 minutes then EDC (259 mg, 1.35 mmol) was added. The reaction was then allowed to stir at room temperature for an additional 8 hours. After 8 hours, the reaction mixture was diluted with dichloromethane (10 mL), washed with saturated NaHCO₃ (2×30 mL), and saturated NaCl solution (30 mL). The organic layer was collected and evaporated to dryness under vacuum. Compound 17c was recovered in 57% yield (0.54 g, 1.2 mmol) after purification by column chromatography on silica using 50% ethyl acetate/hexanes as the eluent (R_{f}=0.3) followed by 75% ethyl acetate/hexanes (R_{f}=0.7). MS: calc. for C₃₄H₄₇Cl₂FN₄O₄S: 696.3; Found: 697 (MH⁺); retention time: 3.110 minutes; Method info: APCI positive ion scan 100-1000 Frag V=80; 100% 0.05% TFA/H₂O to 90% ACN/0.05% TFA over 2 min, 2.5 min run, ODS-AQ column.

### Step 17D: 2-Fluorophenyl Pyrrolidine 17d

Boc-pyrrolidine sulfinamide 17c (0.55 g, 0.78 mmol) was dissolved in MeOH (10 mL) and HCl (2M in ether, 507 μL, 1.01 mmol) was added to the reaction vial. The reaction mixture was allowed to stir at room temperature for 1 hour or until all of the starting material had been consumed (monitored by TLC). Nitrogen gas was then bubbled through the reaction mixture to evaporate residual HCl then the remaining solvent was removed in vacuo. The residue was dissolved in dichloromethane (10 mL), washed with saturated NaHCO₃ (3×20 mL) and saturated NaCl (20 mL). The organic layer was collected, dried over anhydrous MgSO₄, filtered, and evaporated to dryness under vacuum. An aliquot of the crude deprotected amine was used for the next step without further purification. The deprotected amino intermediate (415 mg, 0.7 mmol) was then dissolved in dichloromethane (3.5 mL) along with HOBt (95 mg, 0.7 mmol), 3-dimethylaminopropionic acid hydrochloride (108 mg, 0.7 mmol), and triethylamine (420 μL, 1.5 mmol). The reaction mixture was allowed to stir at room temperature for 10 minutes then EDC (134 mg, 0.7 mmol) was added. The reaction was then allowed to stir at room temperature for an additional 8 hours. After 8 hours, the reaction mixture was diluted with dichloromethane (5 mL) and washed with saturated NaHCO₃ (2×15 mL). The organic layer was collected and evaporated to dryness under vacuum. The residue was dissolved in 1:1 TFA/DCM (5 mL) and stirred at room temperature for 30 minutes. The reaction mixture was then evaporated to dryness under a stream on nitrogen to give 17d (0.23 g, 0.39 mmol) in 50% yield over 3 steps. A small portion was purified by preparative HPLC (the remaining portion was used for the next step without any further purification). Compound 17d was recovered as the TFA salt in 19% yield. MS: calc. for C₃₀H₄₀C₁₂FN₅O₂: 591.3; Found: 592 (MH⁺); retention time: 4.743 minutes; Method info: APCI positive ion scan 100-1000 Frag V=80; 95% 0.025% TFA/H₂O to 95% ACN/0.025% TFA over 13 min, 15.5 min run, ODS-AQ column.

### Step 17E: Dimethylamino Acetyl Pyrrolidine 17-1

2-Fluorophenyl pyrrolidine 17d (59 mg, 0.1 mmol) was dissolved in dichloroethane (0.5 mL) along with triethylamine (14 μL, 0.1 mmol) and acetic anhydride (11 μL, 0.1 mmol). The reaction mixture was allowed to stir at room temperature for 8 hours then was diluted with dichloromethane (2 mL). The organic layer was washed with saturated NaHCO₃ (3×5 mL), saturated NaCl (5 mL), and solvent was evaporated under a stream of nitrogen. The residue was purified by preparative HPLC to give compound 17-1 as the TFA salt in 26% yield. MS: calc. for C₃₂H₄₂Cl₂FN₅O₃: 633.3; Found: 634 (MH⁺); retention time: 5.942 minutes; Method info: APCI positive ion scan 100-1000 Frag V=80; 95% 0.025% TFA/H₂O to 95% ACN/0.025% TFA over 13 min, 15.5 min run, ODS-AQ column.

By the above procedures, the compounds of the following Table 17 were prepared.

TABLE 17

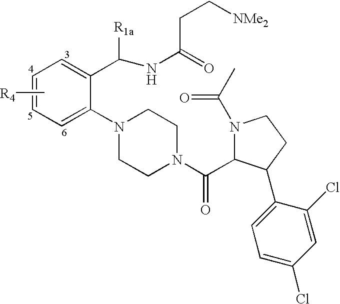

| Cpd | R₄ | R₁ₐ | MW | (MH⁺) |
|---|---|---|---|---|
| 17-1 | 6-F | i-Pr | 633.6 | 634 |
| 17-2 | 4-Cl | i-Bu | 664.1 | 665 |

### Example 18

### {4-[2-((S)-1-AMINO-3-METHYL-BUTYL)-4-TRIFLUOROMETHYL-PHENYL]-PIPERAZIN-1-YL}-[1-SEC-BUTYL-4-(4-METHOXY-PHENYL)-PYRROLIDIN-3-YL]-METHANONE

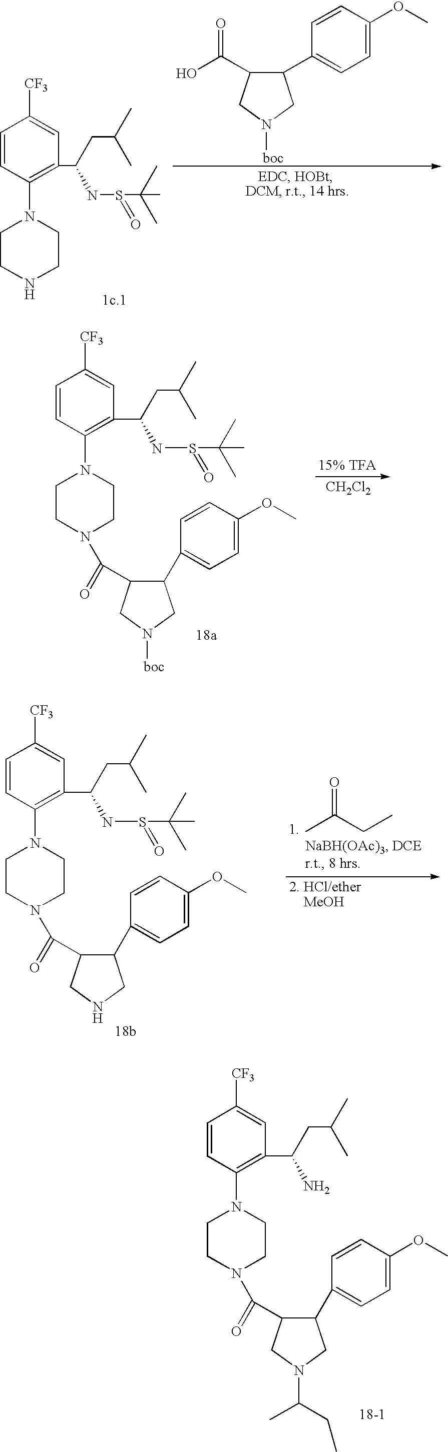

### Step 18A: Compound 18a

To a dichloromethane (4 mL) solution of 2-[1-piperazinyl]-1-[1S-(S-t-butanesulfinamido)-3-methylbutyl]-5-trifluoromethylbenzene 1c (0.643 g, 2.00 mmol) at room temperature, was added 1-[(tert-butyl)oxycarbonyl]-4-(4-methoxyphenyl)pyrrolidine-3-carboxylic acid (0.838 g, 2.00 mmol) and HOBt (0.324 g, 2.40 mmol). The solution stirred for 20 minutes under nitrogen and then EDC (0.458 g, 2.40 mmol) was added. The reaction continued to stir for 14 hours. The mixture was then diluted with dichloromethane (10 mL) and washed with sat. NaHCO₃ (10 mL) solution and then saturated NaCl (10 mL) solution. The organic layer was collected and dried over anhydrous Na₂SO₄. Organic solvent was removed in vacuo to afford the product as a yellow solid. The product was further purified by column chromatography on silica using 1:1 hexane/ethyl acetate as the eluents. Organic solvents were concentrated in vacuo to afford 0.380 g (30% yield) of 18a as a light yellow solid.

### Step 18B: Compound 18b

Boc-protected pyrrolidine 18a (1.11 g, 1.54 mmol) was dissolved in dichloromethane (15 mL), placed under nitrogen, and then treated with TFA (2.50 mL). The reaction stirred at room temperature for 30 minutes. The reaction was then neutralized with saturated NaHCO₃ solution. The organic layer was collected, dried over anhydrous Na₂SO₄, and solvent removed in vacuo to afford 18b as a light yellow solid in quantitative yield.

### Step 18C: Compound 18-1

A 0.10 M solution of the deprotected pyrrolidine 18b (0.062 g, 0.10 mmol) was prepared in dichloroethane and transferred to a 4 dram vial. Methyl ethyl ketone (0.008 mL, 0.10 mmol) and acetic acid (0.060 mL, 0.10 mmol) was added. The vial was capped, allowed to stir at room temperature for 15 minutes, and then treated with NaBH(OAc)₃. The reaction continued to stir for 8 hours. The reaction was then diluted with dichloromethane (1 mL) and washed with saturated NaHCO₃ (1 mL). The organic layer was collected and solvents reduced by a stream of nitrogen. The residue (0.068 g, 0.10 mmol) above was dissolved in MeOH (1 mL) and then treated with 2M HCl in diethyl ether (0.20 mmol). The reaction was capped and allowed to stir for 20 minutes at room temperature. The organic solvents were reduced under a stream of nitrogen and the residue was suspended in methanol (1 mL) and purified by prep HPLC to give 42 mg of compound 18-1 (75% yield). LCMS (tᵣ, 7.030) 561(MH⁺)

### Example 19

### N-[(S)-1-(2-{4-[4-(4-CHLORO-PHENYL)-1-CYCLOHEXYL-PYRROLIDINE-3-CARBONYL]-PIPERAZIN-1-YL}-3-FLUORO-PHENYL)-2-METHYL-PROPYL]-3-DIMETHYLAMINO-PROPIONAMIDE

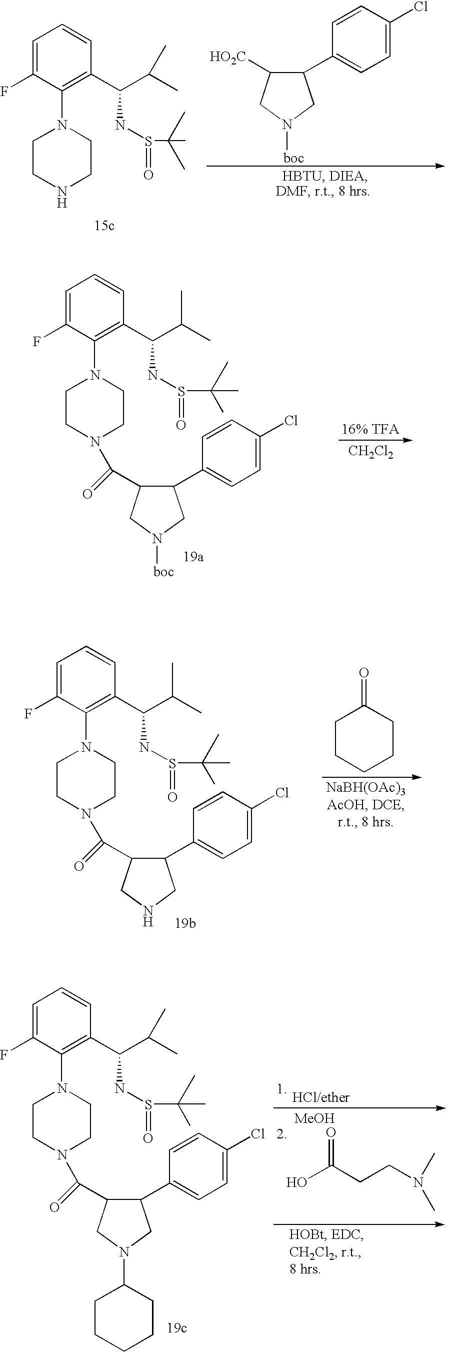

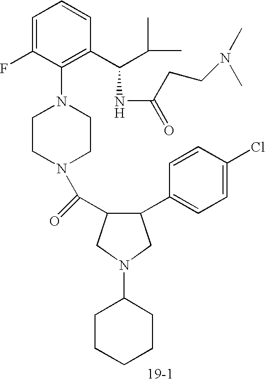

### Step 19A: Compound 19a

To a DMF (6 mL) solution of 1-[(tert-butyl)oxycarbonyl]-4-(4-chlorophenyl)pyrrolidine-3-carboxylic acid (0.448 g, 1.50 mmol) was added HBTU (0.569 g, 1.50 mmol) along with DIEA (0.522 mL, 3.00 mmol) at room temperature. The mixture was placed under nitrogen and allowed to stir for 40 minutes. 2-[1-piperazinyl]-1-[1S-(S-t-butanesulfinamido)-2-methylpropyl]-3-fluorobenzene 15c, which was dissolved in 1 mL DMF, was added and the reaction stirred for 8 hours. The mixture was then diluted with ethyl acetate (12 mL) and washed with saturated NaHCO₃ (2×12 mL) and then with saturated NaCl solution (2×12 mL). The organic layer was collected, dried over anhydrous Na₂SO₄, and solvent removed in vacuo to afford 0.620 g (62% yield) of 19a as a light yellow solid. No further purification was needed.

### Step 19B: Compound 19b

The Boc-protected pyrrolidine 19a (0.786 g, 1.18 mmol), under nitrogen atmosphere, was dissolved in dichloromethane (12 mL), and treated with TFA (1.90 mL). The reaction stirred at room temperature until TLC showed no starting material (approximately 1 hour). The reaction was neutralized with saturated NaHCO₃ and the organic layer separated, dried over anhydrous Na₂SO₄, and solvent removed in vacuo to afford 19b as a light yellow solid in quantitative yield.

### Step 19C: Compound 19c

A 0.10 M solution of the deprotected pyrrolidine 19b (0.056 g, 0.10 mmol) was prepared in dichloroethane and transferred to a 4 dram vial along with cyclohexanone (0.011 mL, 10 mmol) and acetic acid (0.060 mL, 0.10 mmol). The vial was capped, allowed to stir at room temperature for 15 minutes, and then treated with NaBH(OAc)₃. The reaction mixture stirred for an additional 8 hours. The mixture was then diluted with dichloromethane (1 mL) and washed with saturated NaHCO₃ solution (1 mL). The organic layer was collected and solvents reduced with a stream of nitrogen to give 19c.

### Step 19D: Compound 19-1

In a capped vial, the sulfinamide 19c (0.066 g, 0.10 mmol) was dissolved in methanol (1 mL) and then treated with 2M HCl in diethyl ether (0.20 mmol). The reaction was capped and stirred for 20 minutes at room temperature. The mixture was then diluted with dichloromethane (1 mL) and neutralized with saturated NaHCO₃. The organic layer was collected, transferred to a 4 dram vial, and then solvent was reduced by a stream of nitrogen to afford the product as a free base. No further purification was needed. The crude intermediate was then dissolved in dichloromethane (1 mL) along with dimethylaminopropionic acid hydrochloride (0.015 g, 0.10 mmol) and HOBt (0.016 g, 0.12 mmol). The reaction mixture 5 was capped and stirred for 15 minutes at room temperature before adding EDC (0.023 g, 0.12 mmol). The reaction continued to stir for 8 hours. The reaction mixture was then diluted with dichloromethane (1 mL) and washed with saturated NaHCO₃ (1 mL). The organic layer was collected and reduced under a stream of nitrogen and the residue was purified by prep HPLC to give 0.034 g of 19-1 (52% yield). LCMS (tᵣ, 4.560) 656 (MH⁺)

### Example 20

### 1-[3-(4-CHLORO-PHENYL)-4-(4-{3-[1-(2-DIMETHYLAMINO-ETHOXY)-2-METHYL-PROPYL]-5-FLUORO-PYRIDIN-2-YL}-PIPERAZINE-1-CARBONYL)-PYRROLIDIN-1-YL]-2,2-DIMETHYL-PROPAN-1-ONE

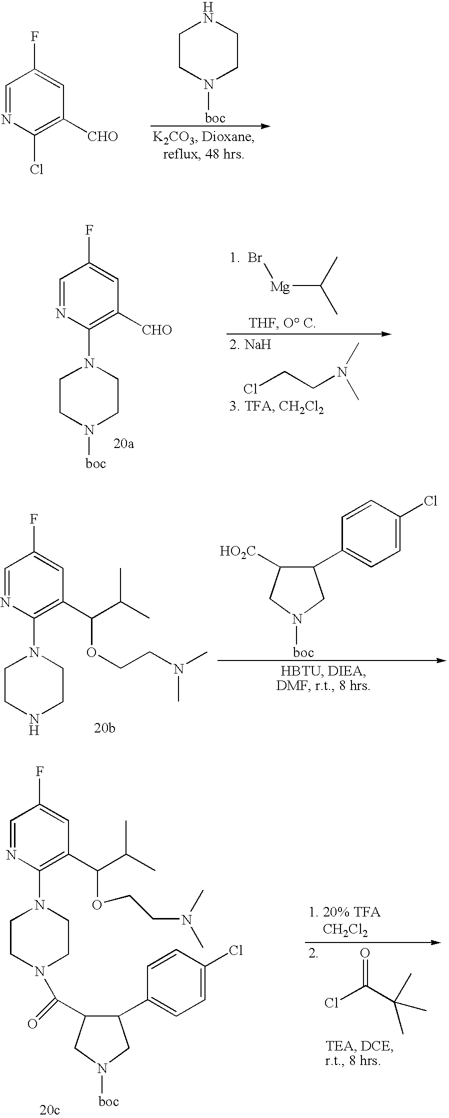

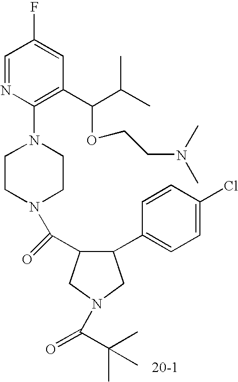

### Step 20A: Compound 20a

In a 250 mL flask, 2-chloro-5-fluoropyridine-3-carboxaldehyde (4.88 g, 31.0 mmol) was dissolved in dioxane (103 mL) along with Boc-piperazine (5.77 g, 31.0 mmol) and potassium carbonate (4.30 g, 31.0 mmol). The reaction was heated to reflux with stirring for 48 hours. The mixture was then diluted with ethyl acetate (100 mL) and washed with saturated NaHCO₃ solution (2×75 mL) and saturated NaCl solution (2×75 mL). The organic layer was collected, dried over anhydrous Na₂SO₄, and then filtered. Solvent was removed in vacuo and the residue was purified by column chromatography on silica using 9:1 hexane/ethyl acetate as the eluent to afford 3.0 g (31% ) of the 20a as a yellow solid.

### Step 20B: Compound 20b

In a 100 mL roundbottom flask, the aldehyde 20a (0.448 g, 1.45 mmol) was dissolved in THF (7 mL), placed under nitrogen, and then cooled to 0° C. Isopropyl Grignard (15% in THF, 11 mL, 1.60 mmol) was added dropwise while maintaining temperature below 0° C. After the addition, the reaction stirred for 20 minutes at 0° C. The reaction was slowly quenched with saturated NH₄Cl solution and then diluted with ethyl acetate (10 mL). The mixture was washed with saturated NaHCO₃ solution (5 mL) and then with saturated NaCl solution (5 mL). The organic layer was extracted, dried over anhydrous Na₂SO₄, filtered, and solvent removed in vacuo to afford an oil in quantitative yield (0.55 g). LCMS (tᵣ, 2.736) MH⁺(354). The oil was dissolved in DMF. NaH (60% in oil) was added and the reaction stirred at room temperature for 1 hour. Then, dimethylamino ethyl chloride was added and the reaction mixture was heated to 60° C. for 14 hours. The reaction mixture was diluted with ethyl acetate (1 mL) and was quenched with H₂O (2 mL). The organic layer was collected and solvent was reduced under a stream of nitrogen. The material was dissolved in dichloromethane (15 mL), placed under nitrogen, and then treated with TFA (3.0 mL). The reaction stirred at room temperature for 30 minutes. The reaction was then neutralized with saturated NaHCO₃ solution and extracted with a 3:1 mixture of chloroform/isopropyl alcohol solution to give 20b.

### Step 20C: Compound 20c

In a 4 dram vial, 1-[(tert-butyl)oxycarbonyl]-4-(4-chlorophenyl)pyrrolidine-3-carboxylic acid (0.033 g, 0.10 mmol) was dissolved in DMF (1 ML) along with HBTU (0.038 g, 0.10 mmol) and DIEA (0.104 ml, 0.20 mmol) at room temperature. The vial was capped and allowed to stir for 15 minutes. The piperazine 20b (0.032 g, 0.10 mmol) was added and the reaction continued to stir for 8 hours at room temperature. The mixture was then diluted with ethyl acetate (1 mL) and washed with saturated NaHCO₃ (2×1 mL) solution and then with saturated NaCl solution (2×1 mL). The organic layer was collected and solvent reduced under a stream of nitrogen to give 20c.

### Step 20D: Compound 20-1

In a 4 dram vial, the Boc-protected pyrrolidine 20c (0.063 g, 0.10 mmol) was treated with 15% TFA in dichloromethane (1 mL). The reaction mix was capped and stirred at room temperature for 30 minutes The reaction mix was diluted with dichloromethane (1 mL) and then neutralized with saturated NaHCO_{3.} The organic layer was collected and solvent was reduced under a stream of nitrogen. Quantitative yield was assumed and no further purification was needed. To a 0.10 M stock solution of the deprotected pyrrolidine 4 (0.059 g, 0.10 mmol) in dichloroethane, was added pivaloyl chloride ( 0.013 mL, 0.10 mmol) and TEA (0.014 mL, 0.10 mmol). The reaction stirred at room temperature for 8 hours then diluted with dichloromethane (1 mL) and washed with saturated NaHCO₃ (1 mL). The organic layer was collected and solvents reduced under a stream of nitrogen. The product was re-suspended in methanol (1 mL) and collected for prep HPLC. LCMS (tᵣ, 5.209) 616 (MH⁺) Yield 0.040 g, 66%

By the above procedures, the compounds of the following Table 20 were prepared.

TABLE 20

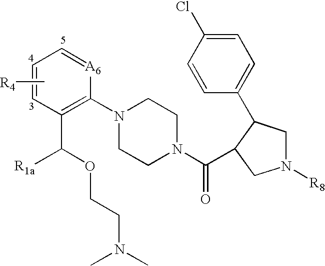

| Cpd | A | R₄ | R₁ₐ | R₈ | MW | (MH⁺) |
|---|---|---|---|---|---|---|
| 20-1 | N | 4-F | iPr | tBuC(O) | 616.2 | |
| 20-2 | N | 4-F | iPr | MeC(O) | 574.1 | |
| 20-3 | N | 4-F | iPr | PhC(O) | 636.2 | |
| 20-4 | N | 4-F | iPr | tBuOC(O) | 632.2 | 632 |
| 20-5 | CR₄ | H | Me | iPr | 527.2 | 527 |
| 20-6 | CR₄ | 6-F | iPr | iPr | 573.2 | 573 |

### Example 21

### [4-(4-CHLORO-PHENYL)-1-ISOPROPYL-PYRROLIDIN-3-YL]-(4-{1-[(4-METHOXY-BENZYLAMINO)-METHYL]-CYCLOPENTYL}-PIPERAZIN-1-YL)-METHANONE

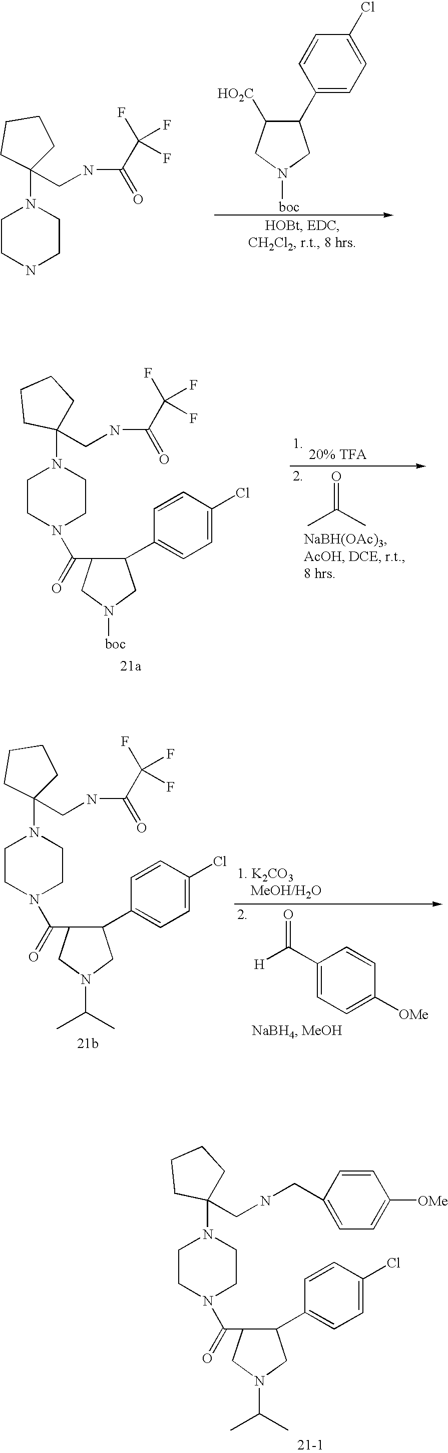

### Step 21A: Compound 21a

1-[1-(Trifluoroacetamidomethyl)cyclohexyl]piperazine (0.340 g, 1.22 mmol), 1-[(tert-butyl)oxycarbonyl]-4-(4-chlorophenyl)pyrrolidine-3-carboxylic acid (0.400 g, 1.22 mmol) and HOBt (0.200 g, 1.47 mmol) were dissolved in dichloromethane (5 mL). The reaction mixture was placed under nitrogen and allowed to stir for 20 minutes. EDC (0.280 g, 1.47 mmol) was added and the mixture continued to stir for 8 hours at room temperature. The reaction mixture was then diluted with dichloromethane (5 mL) and was washed with saturated NaHCO₃ solution (5 mL) and saturated NaCl solution (5 mL). The organic layer was collected, dried over anhydrous NaSO₄, and then solvent removed in vacuo to afford 21a as a yellow solid in quantitative yield. No further purification was needed. LCMS (t_{R}, 2.528) 587 (MH⁺).

### Step 21B: Compound 21b

The Boc-protected pyrrolidine 21a (0.714 g, 1.22 mmol) was dissolved in dichloromethane (12 mL), placed under nitrogen, and then treated with TFA (2.4 mL). The mixture was stirred at room temperature for 1 hour. The mixture was neutralized with saturated NaHCO₃ and the organic layer was separated, dried over anhydrous Na₂SO₄, and the solvent removed in vacuo to give a light yellow solid in quantitative yield. The light yellow solid (0.561 g, 1.15 mmol) was dissolved in dichloroethane along with acetone (0.084 mL) and acetic acid (0.065 mL, 1.15 mmol). The reaction mixture was placed under nitrogen and the mixture stirred for 20 minutes before adding NaBH(OAc)₃ (0.341 g, 1.60 mmol). The mixture continued to stir for 8 hours at room temperature. The reaction mix was diluted with dichloromethane (12 mL) and was washed with saturated NaHCO₃ (12 mL) and saturated NaCl (12 mL). The organic layer was collected and dried over anhydrous Na₂SO₄. Solvent was removed in vacuo to give 21b (0.591 g, 91% ) as a yellow solid.

### Step 21C: Compound 21c

Compound 21b (0.591 g, 1.12 mmol) is dissolved in a 19:1 mixture of MeOH/H₂O (17 mL). Potassium carbonate (3.70 g, 27.3 mmol) was added and the mixture was heated at 65° C. for 8 hours. The mix was diluted with dichloromethane (30 mL) and was washed with water (2×10 mL). The organic layer was collected and solvent was removed in vacuo to give a residue which was dissolved in methanol to make a 0.10 M stock solution. 1 mL of the stock solution was transferred to a 4 dram vial. P-Anisaldehyde was added (0.012 mL, 0.10 mmol) and the vial was capped and allowed to stir at room temperature for 15 minutes before adding sodium triacetoxyborohydride (0.06 g, 0.14 mmol). The reaction was stirred for 1 hour, diluted with dichloromethane (1 mL) and quenched with saturated NaHCO₃. The organic layer was collected and solvent reduced under a stream of nitrogen. Methanol (1 mL) was added and purification by prep HPLC gave 21-1 in 99% yield. LCMS (t_{R}, 4.471) 553 (MH⁺).

By the above procedures, the compounds of the following Table 21 were prepared.

TABLE 21

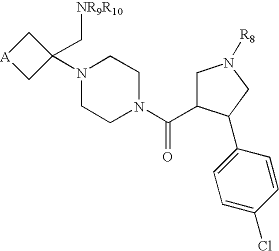

| Cpd | A | R₉ | R₁₀ | R₈ | MW | (MH⁺) |
|---|---|---|---|---|---|---|
| 21-1 | (CH₂)₂ | 4-MeO-Bn | H | iPr | 553.2 | 553 |
| 21-2 | CH₂ | H | H | iPr | 419.0 | 419 |
| 21-3 | CH₂ | cyclopentyl | H | iPr | 487.1 | 487 |
| 21-4 | CH₂ | cyclohexyl | H | iPr | 501.2 | 501 |
| 21-5 | CH₂ | 4-MeO-Bn | H | iPr | 539.2 | 539 |
| 21-6 | CH₂ | 4-PyCH₂ | H | iPr | 510.1 | 510 |
| 21-7 | (CH₂)₂ | H | H | iPr | 433.0 | 433 |
| 21-8 | (CH₂)₂ | cyclopentyl | H | iPr | 501.2 | 501 |
| 21-9 | (CH₂)₂ | cyclohexyl | H | iPr | 515.2 | 515 |
| 21-10 | (CH₂)₂ | 4-F-Bn | H | iPr | 541.2 | 541 |
| 21-11 | (CH₂)₂ | 4-PyCH₂ | H | iPr | 524.1 | 553 |
| 21-12 | (CH₂)₃ | 2-PyCH₂ | H | iPr | 538.2 | 524 |
| 21-13 | (CH₂)₃ | 3-PyCH₂ | H | iPr | 538.2 | 538 |
| 21-14 | (CH₂)₃ | 4-PyCH₂ | H | iPr | 538.2 | 538 |
| 21-15 | (CH₂)₃ | H | H | iPr | 447.1 | 447 |
| 21-16 | (CH₂)₃ | cyclohexylCH₂ | H | iPr | 543.2 | 543 |
| 21-17 | (CH₂)₃ | 1-Me₂- | H | iPr | 540.2 | |
| | | pyrrolylCH₂ | | | | |
| 21-18 | (CH₂)₃ | 2-furanylCH₂ | H | iPr | 527.1 | 527 |
| 21-19 | (CH₂)₃ | Bn | H | iPr | 537.2 | 537 |
| 21-20 | (CH₂)₃ | 2-F-Bn | H | iPr | 555.2 | 555 |
| 21-21 | (CH₂)₃ | 2-Cl-Bn | H | iPr | 571.6 | 571 |
| 21-22 | (CH₂)₃ | 2-MeO-Bn | H | iPr | 567.2 | 567 |
| 21-23 | (CH₂)₃ | 2-CF₃Bn | H | iPr | 605.2 | 605 |
| 21-24 | (CH₂)₃ | 3-MeO-Bn | H | iPr | 567.2 | 567 |
| 21-25 | (CH₂)₃ | 3-MeO-Bn | 3-MeOBn | iPr | 687.4 | 687 |
| 21-26 | (CH₂)₃ | 4-MeO-Bn | H | iPr | 567.2 | 567 |
| 21-27 | (CH₂)₃ | 4-MeO-Bn | 4-MeOBn | iPr | 687.4 | |
| 21-28 | (CH₂)₃ | 2-thienylCH₂ | H | iPr | 543.2 | 543 |
| 21-29 | (CH₂)₃ | 2-thienylCH₂ | 2-thienylCH₂ | iPr | 639.4 | 639 |
| 21-30 | (CH₂)₃ | 3-thienylCH₂ | H | iPr | 543.2 | 543 |
| 21-31 | (CH₂)₃ | 3-thienylCH₂ | 3-thienylCH₂ | iPr | 639.4 | 639 |
| 21-32 | (CH₂)₃ | 2-PyCH(Me) | H | iPr | 552.2 | 552 |
| 21-33 | (CH₂)₃ | 4-CF₃-Bn | H | iPr | 605.2 | 605 |
| 21-34 | (CH₂)₃ | 4-CF₃-Bn | 4-CF₃Bn | iPr | 763.3 | 765 |
| 21-35 | (CH₂)₃ | 3-furanylCH₂ | H | iPr | 527.1 | 527 |
| 21-36 | (CH₂)₃ | 3-furanylCH₂ | 3-furanylCH₂ | iPr | 607.2 | 607 |
| 21-37 | (CH₂)₃ | 2-CN-Bn | H | iPr | 562.2 | |
| 21-38 | (CH₂)₃ | 2-pyrrolylCH₂ | H | iPr | 526.2 | |
| 21-39 | (CH₂)₃ | 2-thiazolylCH₂ | H | iPr | 544.2 | 544 |
| 21-40 | (CH₂)₃ | PhCH₂CH₂ | H | iPr | 551.2 | |
| 21-41 | (CH₂)₃ | MeOCH₂CH(Me) | H | iPr | 519.2 | 519 |
| 21-42 | (CH₂)₃ | 1-Me-2- | H | iPr | 590.3 | 590 |
| | | indolylCH₂ | | | | |
| 21-43 | (CH₂)₃ | 2-CF₃O-Bn | H | iPr | 621.2 | 621 |
| 21-44 | (CH₂)₃ | 2,3-di-Me-4- | H | iPr | 555.2 | 555 |
| | | pyrazolylCH₂ | | | | |
| 21-45 | (CH₂)₃ | 4-F-Bn | H | iPr | 555.2 | 555 |
| 21-46 | (CH₂)₃ | 4-C1-Bn | H | iPr | 571.6 | 571 |
| 21-47 | (CH₂)₃ | 4-AcNHBn | H | iPr | 594.2 | 594 |
| 21-48 | (CH₂)₃ | 2-PyCH₂CH₂ | H | iPr | 552.2 | 55₂ |
| 21-49 | (CH₂)₃ | 3-F-Bn | H | iPr | 555.2 | 555 |
| 21-50 | (CH₂)₃ | 4-NO2-Bn | H | iPr | 582.2 | 582 |
| 21-51 | (CH₂)₃ | 4-Me₂N-Bn | H | iPr | 580.3 | 580 |
| 21-52 | (CH₂)₃ | 4-MeO-Bn | Me | iPr | 581.2 | 581 |
| 21-53 | (CH₂)₃ | 4-MeO-Bn | Et | iPr | 595.3 | 595 |
| 21-54 | (CH₂)₃ | 4-MeO-Bn | iBu | iPr | 623.3 | 623 |
| 21-55 | (CH₂)₃ | 4-PyCH₂ | Me | iPr | 552.2 | 552 |
| 21-56 | (CH₂)₃ | 4-PyCH₂ | Et | iPr | 566.2 | 566 |
| 21-57 | (CH₂)₃ | 4-PyCH₂ | iBu | iPr | 594.3 | 594 |
| 21-58 | (CH₂)₃ | 4-(N- | H | iPr | 554.2 | 554 |
| | | oxide)PyCH₂ | | | | |
| 21-59 | (CH₂)₃ | 2,4-di-MeO-Bn | H | iPr | 597.2 | 597 |
| 21-60 | (CH₂)₃ | 2-F-4-MeO-Bn | H | iPr | 585.2 | |
| 21-61 | (CH₂)₃ | 2- | H | iPr | 527.2 | 527 |
| | | imidazolylCH₂ | | | | |
| 21-62 | (CH₂)₃ | 4- | H | iPr | 527.2 | 527 |
| | | imidazolylCH₂ | | | | |
| 21-63 | (CH₂)₃ | 3-F-4-MeOBn | H | iPr | 585.2 | 585 |
| 21-64 | (CH₂)₃ | 4-MeOC(O)-Bn | H | iPr | 595.2 | 595 |
| 21-65 | (CH₂)₃ | 4-MeS-Bn | H | iPr | 583.3 | |
| 21-66 | (CH₂)₃ | 3,4-CH₂O2Bn | H | iPr | 581.2 | 581 |
| 21-67 | (CH₂)₃ | 2,4-di-F-Bn | H | iPr | 573.2 | 573 |
| 21-68 | (CH₂)₃ | 4-iPrOBn | H | iPr | 595.3 | 595 |
| 21-69 | (CH₂)₃ | 2-F-4-MeO-Bn | H | iPr | 585.2 | 585 |
| 21-70 | (CH₂)₃ | 4-MeO-Bn | H | iPr | 553.2 | |
| 21-71 | (CH₂)₃ | 5- | H | iPr | 539.2 | 539 |
| | | pyrimidylCH₂ | | | | |
| 21-7 | (CH₂)₃ | 1-Me₂ | H | iPr | 541.2 | 541 |
| | | imidazolylCH₂ | | | | |
| 21-73 | (CH₂)₃ | 3-F-4- | H | iPr | 599.2 | |
| | | MeOC₆H₃CH(Me) | | | | |
| 21-74 | (CH₂)₃ | Bn | H | CH(Me)CH₂OMe | 567.2 | 567 |
| 21-75 | (CH₂)₃ | Bn | H | cyclopentyl | 563.2 | 563 |
| 21-76 | (CH₂)₃ | Bn | H | cyclohexyl | 577.3 | 578 |
| 21-77 | (CH₂)₃ | Bn | H | 4-pyranyl | 579.2 | 579 |
| 21-78 | (CH₂)₃ | Bn | H | Bn | 585.2 | 585 |
| 21-79 | (CH₂)₃ | 4-PyCH₂ | H | Bn | 586.2 | 586 |
| 21-80 | (CH₂)₃ | 4-PyCH₂ | H | H | 496.1 | 495 |
| 21-81 | CH₂OCH₂ | H | H | iPr | 449.0 | 449 |
| 21-82 | CH₂OCH₂ | cyclohexyl | H | iPr | 531.2 | 531 |
| 21-83 | CH₂OCH₂ | 4-F-Bn | H | iPr | 557.2 | 557 |
| 21-84 | CH₂OCH₂ | 2,3-di-Me-4- | H | iPr | 557.2 | 557 |
| | | pyrazolylCH₂ | | | | |
| 21-85 | (CH₂)₃ | 4-PyCH₂ | H | C(O)Et | 552.2 | 551 |
| 21-86 | (CH₂)₃ | 4-PyCH₂ | H | C(O)Me | 538.1 | 537 |
| 21-87 | (CH₂)₃ | Bn | H | C(O)iPr | 565.2 | 565 |
| 21-88 | (CH₂)₃ | Bn | H | C(O)Me | 537.1 | 537 |
| 21-89 | (CH₂)₃ | Bn | H | C(O)Et | 551.2 | 551 |
| 21-90 | (CH₂)₃ | Bn | H | C(O)Pr | 565.2 | 565 |
| 21-91 | (CH₂)₃ | Bn | H | C(O)CH₂NHMe | 566.2 | 566 |
| 21-92 | (CH₂)₃ | Bn | H | C(O)CH(Me)NH₂ | 566.2 | 566 |
| 21-93 | (CH₂)₃ | Bn | H | C(O)CH₂CH2NH₂ | 566.2 | 566 |
| 21-94 | (CH₂)₃ | Bn | H | C(O)CH(iPr)NH₂ | 594.2 | 594 |

### Example 22

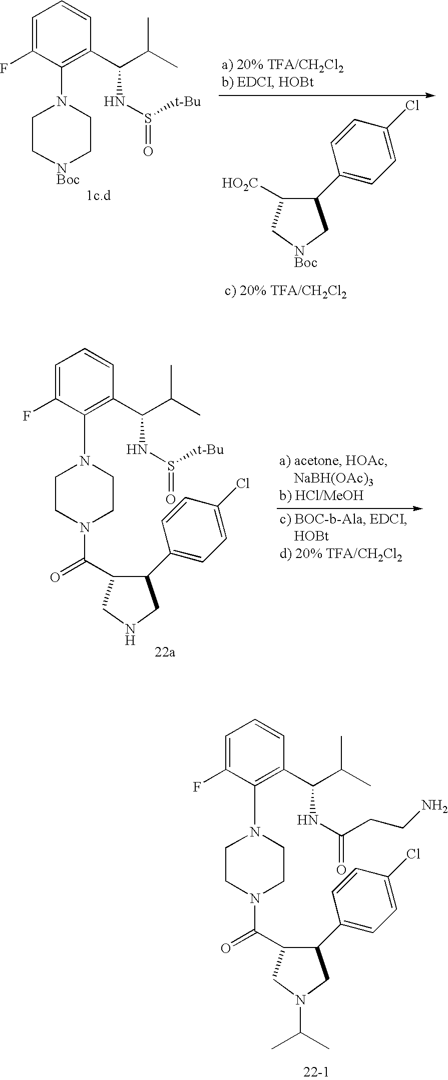

### Step 22A: Synthesis of Pyrrolidine 22a

To a dichloromethane (25 mL) solution of BOC-piperazine 1c.d (1.400 g, 3.072 mmol) was added trifluoroacetic acid (6.0 mL) at room temperature and the mixture was stirred for 50 minutes. The reaction mixture was neutralized with saturated aqueous NaHCO₃ solution and extracted with EtOAc (2×100 mL). The organic layer was dried over Na₂SO₄ and evaporated to provide the piperazine as white foam, which was dissolved in DMF/CH₂Cl₂ (1:3, 30 mL). To this solution was added NaHCO₃ (774 mg, 9.21 mmol), 1-[(tert-butyl)oxycarbonyl]-4-(4-chlorophenyl)pyrrolidine-3-carboxylic acid (mix of trans isomers, 1.000 g, 3.072 mmol), HOBt (498 mg, 3.69 mmol), EDCI (707 mg, 3.69 mmol) sequentially. The reaction mixture was stirred overnight at room temperature. The mixture was diluted with EtOAc (100 mL), washed with 5% aqueous HCl (30 mL), saturated aqueous NaHCO₃ (25 mL), brine (25 mL), and dried (Na₂SO₄). The solution was concentrated in vacuo to provide crude product, which was purified by flash column chromatography (40˜50% EtOAc in Hexanes) to provide pure a white foam (1.772 g, 87% ). This white foam (1.772 g, 2.673 mmol) was dissolved in dichloromethane (25 mL) and treated with trifluoroacetic acid (6.0 mL) at room temperature and the mixture was stirred for 50 minutes. The reaction mixture was neutralized with saturated aqueous NaHCO₃ solution and extracted with EtOAc (2×100 mL). The organic layer was dried over Na₂SO₄ and evaporated to provide the pyrrolidine 22a as light yellow foam (1.460 g, 97% ).

### Step 22B: Synthesis of Substituted Pyrrolidine 22-1

To a dichloromethane (4 mL) solution of pyrrolidine 22a (270 mg, 0.407 mmol) was added acetone (60 μL, 0.814 mmol) and acetic acid (47 μL, 0.814 mmol) at room temperature followed by the addition of sodium triacetoxyborohydride (173 mg, 0.814 mmol). The reaction was monitored by LC/MS. The reaction mixture was diluted with EtOAc (50 mL) and washed with saturated aqueous NaHCO₃ solution (20 mL). The organic solution was dried over Na₂SO₄ and evaporated to provide isopropyl pyrrolidine. A portion of this isopropyl pyrrolidine (100 mg, 0.165 mmol) was dissolved in MeOH (2 mL) and treated with HCl (62 μL 4 N HCl in dioxane, 0.248 mmol). The mixture was stirred for 1 h at room temperature. The excess of HCl and solvent were removed in vacuo. The solid residue was dissolved in DMF/CH₂Cl₂ (1:3, 2 mL). To this solution was added NaHCO₃ (41.6 mg, 0.495 mmol), BOC-β-alanine (37.4 g, 0.198 mmol), HOBt (44.6 mg, 0.330 mmol), and EDCI (63.3 mg, 0.330 mmol), sequentially. The reaction mixture was stirred overnight at room temperature. The mixture was diluted with EtOAc (50 mL), washed with saturated aqueous NaHCO₃ (20 mL), brine (20 mL), and dried (Na₂SO₄). The solution was concentrated in vacuo to provide crude product, which was treated in dichloromethane/TFA (1:1 mixture, 5 mL) for 1 hour. The excess of TFA and solvent were removed in vacuo. The resulting oil was purified by flash column chromatography (10˜17% MeOH in dichloromethane) to provide 22-1 as light yellow foam (a mixture of two diastereomers, 63 mg, 67% ). LCMS 572 (MH⁺), t_{R}=1.597.

### Example 23

### ENANTIOMERS OF 1-[(TERT-BUTYL)OXYCARBONYL]-4-(4-CHLOROPHENYL)PYRROLIDINE-3-CARBOXYLIC ACID

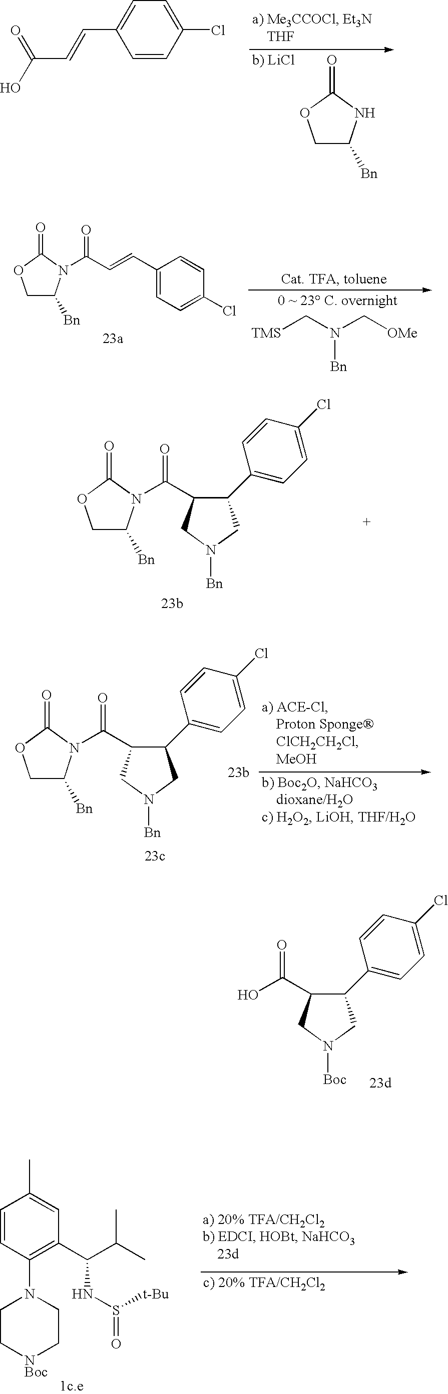

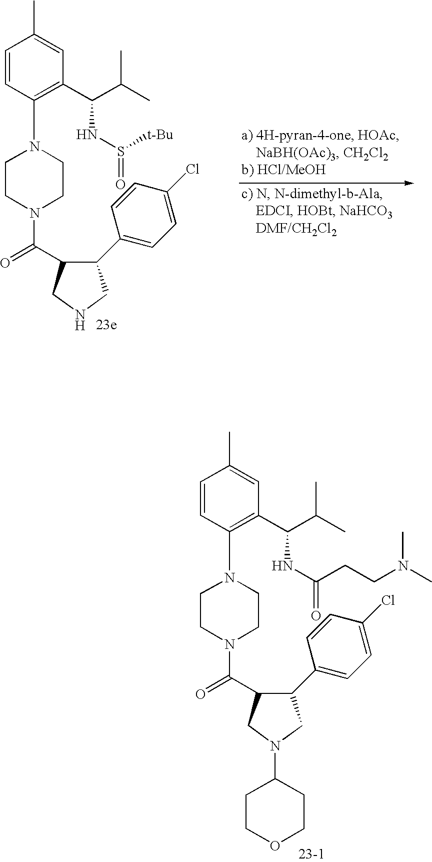

### Step 23A: Compound 23a

To a THF (300 mL) solution of 4-chlorocinnamic acid (10.00 g, 54.76 mmol) was added triethylamine (15.3 mL, 110 mmol) at −20° C. followed by the addition of trimethylacetic chloride (8.1 mL, 66 mmol). White precipitate formed several minutes later. The reaction mixture was stirred for 2 h at −20° C. followed by the addition of lithium chloride (4.66 g, 110 mmol) and (R)-4-benzyl-2-oxazolidinone (11.65 g, 65.72 mmol). The reaction mixture was stirred overnight and the reaction temperature rose naturally to room temperature. The solvent was removed in vacuo. The residue was diluted with EtOAc (200 mL) and washed with saturated aqueous NaHCO₃ solution (100 mL). The organic layer was dried over Na₂SO₄ and evaporated to provide a white solid which was recrystallized in EtOAc/Hexanes to give 23a as fluffy white needles (17.4 g, 93% ).

### Step 23B: Compound 23b

To a toluene (100 mL) suspension solution of 23a (6.900 g, 20.19 mmol) was added N-Benzyl-N-(methoxymethyl)-N-trimethylsilylmethylamine (8.1 mL, 31 mmol) followed by the dropwise addition of a toluene (2 mL) solution of TFA (0.30 mL, 4.0 mmol) at 0° C. The reaction mixture was stirred overnight and the reaction temperature rose to room temperature. The reaction mixture was washed with saturated aqueous NaHCO₃ (20 mL) and brine (20 mL). The solvent was evaporated in vacuo. A mixture of two diastereomers was separated by flash column chromatography (4:0.5:5.5-6.5:1.5:2 of dichloromethane, EtOAc and hexanes) to give less polar diastereomer 23b as a white solid (3.853 g, 40% ) and polar diastereomer 23c (4.436 g 46% , structure was confirmed by x-ray crystallography).

### Step 23C: Compound 23d

To a 1,2-dichioroethane (110 mL) solution of 23b (5.243 g, 11.04 mmol) and Proton Sponge® (1.183 g, 5.520 mmol) in a 250 mL round bottom flask was added 1-chloroethyl chloroformate (ACE-Cl, 2.4 mL, 22 mmol) drop wise at 0° C. The ice bath was removed and the reaction mixture was refluxed until no 23b was detected (about 1 h). Two thirds of 1,2-dichloroethane was removed in vacuo. 100 mL of MeOH was added into the reaction flask and the reaction mixture was refluxed for a half hour. The reaction solvents were removed in vacuo to give a white solid residue. The solid residue was dissolved in 100 mL of water/dioxane (1:1). The solution was treated with NaHCO₃ (20 mL) and brine (1.855 g, 22.08 mmol) and di-tert-butyl dicarbonate (3.614 g, 16.56 mmol) and stirred for overnight. The solvents were evaporated in vacuo. The crude product was purified by flash plug column chromatography (30% EtOAc in hexanes) to give Boc-pyrrolidine as small needles (5.14 g, 97% ). To a water/THF (100 mL) solution of the Boc-pyrrolidine (5.325 g, 10.98 mmol) in a 250 mL round bottom flask was added an aqueous H₂O₂/LiOH solution drop wise at 0° C. The aqueous H₂O₂/LiOH solution was prepared by adding H₂O₂ (3.1 mL, 55 mmol) to an aqueous solution (10 mL) of LiOH.H₂O (1.152 g, 27.45 mmol). The reaction mixture was stirred for 2 h at 0° C. followed by adding of aqueous Na₂SO₃ solution (6.920 g, 54.90 mmol in 50 mL water) and stirring for 2 h at 0° C. The reaction solvent THF was removed in vacuo. The remaining aqueous mixture was extracted with CH₂Cl₂ (4×50 mL). The combined CH₂Cl₂ solution was washed with 10% aqueous Na₂CO₃ solution (4×50 mL). The combined aqueous mixture was extracted with EtOAc (4×100 mL). The EtOAc solution was dried over Na₂SO₄, and evaporated in vacuo to give pyrrolidine acid 23d as white powder (3.43 g, 96% ).

### Step 23D: Compound 23e

To a dichloromethane (4.0 mL) solution of BOC-piperazine 1c.e (200 mg, 0.443 mmol) was added trifluoroacetic acid (1.0 mL) at room temperature and the mixture was stirred for 50 minutes. Saturated aqueous NaHCO₃ solution was added and the mix was extracted with EtOAc (2×25 mL). The organic layer was dried over Na₂SO₄ and evaporated to provide the piperazine as white foam, which was dissolved in DMF/methylene chloride (1:2, 4.5 mL). To this solution was added NaHCO₃ (111.6 mg, 1.329 mmol), (S,R)-1-[(tert-butyl)oxycarbonyl]-4-(4-chlorophenyl)pyrrolidine-3-carboxylic acid 23d (144.3 mg, 0.4429 mmol), HOBt (119.7 mg, 0.8857 mmol), EDCI (169.8 mg, 0.8857 mmol) sequentially. The reaction mixture was stirred overnight at room temperature. The mixture was diluted with EtOAc (40 mL), washed with 5% aqueous HCl (15 mL), saturated aqueous NaHCO₃ (25 mL), brine (25 mL), and dried (Na₂SO₄). The solution was concentrated in vacuo to provide material which was purified by flash column chromatography (40˜60% EtOAc in Hexanes) to provide BOC-pyrrolidine as white foam (257 mg, 88% ). This white foam (148.6 mg, 0.2254 mmol) was dissolved in dichloromethane (2.0 mL) and treated with trifluoroacetic acid (0.5 mL) at room temperature and the mixture was stirred for 30 minutes. The reaction mixture was basified with saturated aqueous NaHCO₃ solution and extracted with EtOAc (2×20 mL). The organic layer was dried over Na₂SO₄ and evaporated to provide pyrrolidine 23e as a light yellow foam (123.5 mg, 98% ) which was used for next step reaction without purification.

### Step 23E: Compound 23-1

To a dichloromethane (2.0 mL) solution of pyrrolidine 23e (123.5 mg, 0.225 mmol) was added tetrahydro-4H-pyran-4-one (41.6 μL, 0.451 mmol) and acetic acid (25.8 μL, 0.451 mmol) at room temperature followed by the addition of sodium triacetoxyborohydride (95.5 mg, 0.451 mmol). The reaction was monitored by LC/MS. The reaction mixture was diluted with EtOAc (25 mL) and washed with saturated aqueous NaHCO₃ solution (15 mL). The organic solution was dried over Na₂SO₄ and evaporated to provide 4H-pyranyl pyrrolidine compound. 4H-pyran-4-yl pyrrolidine compound (61.6 mg, 0.0956 mmol) was dissolved in MeOH (3.0 mL) and treated with HCl (35.9 μL 4 N HCl in dioxane, 0.144 mmol). The mixture was stirred for 40 minutes at room temperature. The excess of HCl and solvent were removed in vacuo. One third of this solid residue (0.0751 mmol) was dissolved in DMF/CH₂Cl₂ (1:3, 2.0 mL). To this solution was added NaHCO₃ (10.7 mg, 0.128mmol), 3-(dimethylamino)-propionic acid (9.8 mg, 0.064 mmol), HOBt (8.6 mg, 0.064 mmol), and EDCI (12.2 mg, 0.0638 mmol), sequentially. The reaction mixture was stirred overnight at room temperature. The mixture was diluted with EtOAc (25 mL), washed with saturated aqueous NaHCO₃ (10 mL), brine (10 mL), and dried (Na₂SO₄). The solution was concentrated in vacuo to provide crude product which was purified by flash column chromatography (10˜17% MeOH in dichloromethane) to provide compound 23-1 as light yellow foam (13 mg, 64% ). LCMS 638 (MH⁺), t_{R}=5.113

By the above procedures, the compounds of the following Table 23 were prepared.

TABLE 23

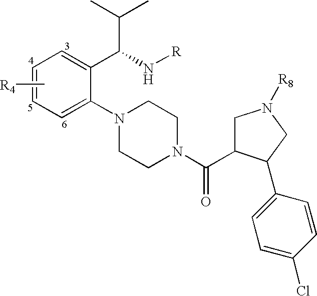

| Cpd | Stereo | R₄ | R | R₈ | MW | (MH⁺) |
|---|---|---|---|---|---|---|
| 23-1 | S,R; R,S | 4-Me | C(O)CH₂ | tetrahydro-4- | 638.3 | 638 |
| | | | CH₂NMe₂ | pyranyl | | |
| 23-2 | R,S; S,R | 4-Me | C(O)CH₂ | tetrahydro-4- | 638.3 | 638 |
| | | | CH₂NMe₂ | pyranyl | | |
| 23-3 | S,R; R,S | 6-F | H | Bn | 549.1 | 549 |
| 23-4 | R,S; S,R | 6-F | H | Bn | 549.1 | 549 |
| 23-5 | S,R; R,S | 4-Me | H | tetrahydro-4- | 539.2 | 539 |
| | | | | pyranyl | | |
| 23-6 | R,S; S,R | 4-Me | H | tetrahydro-4- | 539.2 | 539 |
| | | | | pyranyl | | |

### Example 24

### {4-[2-((S)-1-AMINO-2-METHYL-PROPYL)-6-FLUORO-PHENYL]-PIPERAZIN-1-YL}-[(3R,4S)-4-(4-CHLORO-PHENYL)-1-CYCLOHEXYL-PYRROLIDIN-3-YL]-METHANONE

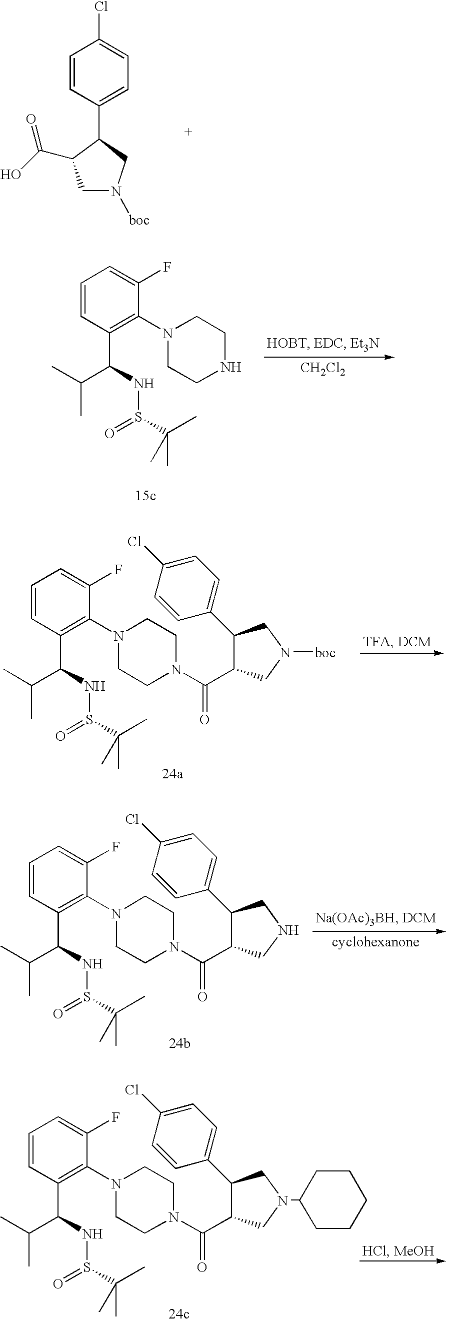

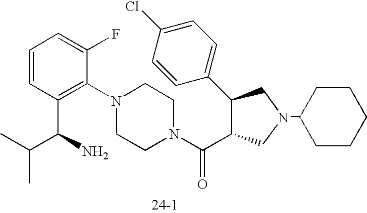

### Step 24A: Compound 24a

To a stirred solution of 4-(4-chlorophenyl)pyrrolidine-1,3-dicarboxylic acid 1-tert-butyl ester (640 mg, 1.97 mmol) and triethylamine (1.1 mL, 8.00 mmol) in CH₂Cl₂ (10 mL), HOBT (405 mg, 3.00 mmol) was added under an inert atmosphere of N₂. After 20 min., EDC (500 mg, 2.60 mmol) was added and the resulting mixture was stirred for another 30 min. A solution of compound 15c (2.1 mmol) was dissolved in CH₂Cl₂ (2 mL) and was added. The resulting solution was allowed to stir overnight. The reaction was quenched with saturated aqueous NaHCO₃ (50 mL) and extracted with CH₂Cl₂. The organics were separated, washed with saturated aqueous NaHCO₃ (50 mL), aqueous HCl (0.1 M, 50 mL) and brine. After drying (MgSO₄) and evaporation, compound 24a was obtained as a tan foam which was used in the next step without further purification.

### Step 24B: Compound 24b

3-(4-Chlorophenyl)-4-(4-{2-fluoro-6-[(S)-2-methyl-1-((S)-2-methylpropane-2-sulfinylamino)propyl]phenyl}piperazine-1-carbonyl)-pyrrolidine-1-carboxylic acid tert-butyl ester 24a (1.32 g, 2.00 mmol) was dissolved in CH₂Cl₂ (20 mL) and treated with TFA (4 mL) for 1 h at room temperature. The reaction mixture was carefully poured onto saturated aqueous NaHCO₃ (200 mL) and extracted with CH₂Cl₂. The organic layers were combined and dried over anhydrous MgSO₄, filtered and concentrated in vacuo to give 24b as a yellow foam.

### Step 24C: Compound 24c

A solution containing 2-methyl-propane-2-sulfinic acid [(S)-1-(2-{4-[4-(4-chloro-phenyl)-pyrrolidine-3-carbonyl]-piperazin-1-yl}-3-fluoro-phenyl)-2-methyl-propyl]-amide 24b (27 mg, 48 μmol) and CH₂Cl₂ (1 mL) was treated with cyclohexanone (26 mg, 265 μmol). The mixture was shaken at room temperature for 1 h and then treated with Na(OAc)₃BH (57 mg, 269 μmol). The resulting heterogeneous mixture was shaken overnight. The reaction was quenched with saturated aqueous NaHCO₃ (3 mL) and extracted with CH₂Cl₂ (10 mL). The organic layer was separated, dried over anhydrous MgSO₄, filtered and evaporated to give 24c which was used in the next step without any further purification.

### Step 24D: Compound 24-1

The crude compound 24c above was dissolved in MeOH (2 mL) and treated with HCl (300 μL of a 2 N solution in Et₂O). After 1 h, the volatiles were removed under a flow of N₂. The crude compound was dissolved in MeOH (1 mL) and purified by preparative HPLC/MS, to give the compound 24-1 as the TFA salt (7 mg, 9 μmol, 19% yield over the last two steps). LRMS m/z 541 (MH⁺).

### Example 25

### {4-[2-((S)-1-AMINO-2-METHYL-PROPYL)-6-FLUORO-PHENYL]-PIPERAZIN-1-YL}-[(3R,4S)-4-(4-CHLORO-PHENYL)-1-CYCLOPROPANECARBONYL-PYRROLIDIN-3-YL]-METHANONE

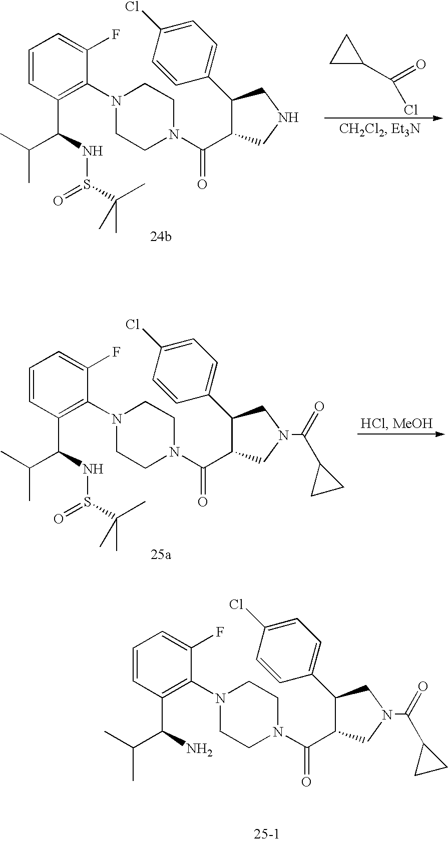

### Step 25A: Compound 25a

A solution containing 2-methyl-propane-2-sulfinic acid [(S)-1-(2-{4-[4-(4-chloro-phenyl)-pyrrolidine-3-carbonyl]-piperazin-1-yl}-3-fluoro-phenyl)-2-methyl-propyl]-amide 24b (27 mg, 48 μmol), CH₂Cl₂ (1 mL) and triethylamine (38 μL, 267 μmol) was treated with cyclopropanecarbonyl chloride (28 mg, 269 μmol). The resulting mixture was shaken at room temperature overnight. The reaction was concentrated under a flow of N₂ and the compound 25a was used in the next step without any further purification.

### Step 25B: Compound 25b

The crude compound 25a above was dissolved in MeOH (2 mL) and treated with HCl (300 μL of a 2 N solution in Et₂O). After 1 h, the volatiles were removed under a flow of N₂. The crude compound was dissolved in MeOH (1 mL) and purified by preparative HPLC/MS, to give the compound 25-1 as the TFA salt (4 mg, 6.2 μmol, 13% over the last two steps). LRMS m/z 527 (MH⁺).

### Example 26

### {4-[2-((S)-1-AMINO-2-METHYL-PROPYL)-4-METHYL-PHENYL]-PIPERAZIN-1-YL}-[(3R,4S)-4-(4-CHLORO-PHENYL)-1-(TETRAHYDRO-PYRAN-4-YL)-PYRROLIDIN-3-YL]-METHANONE

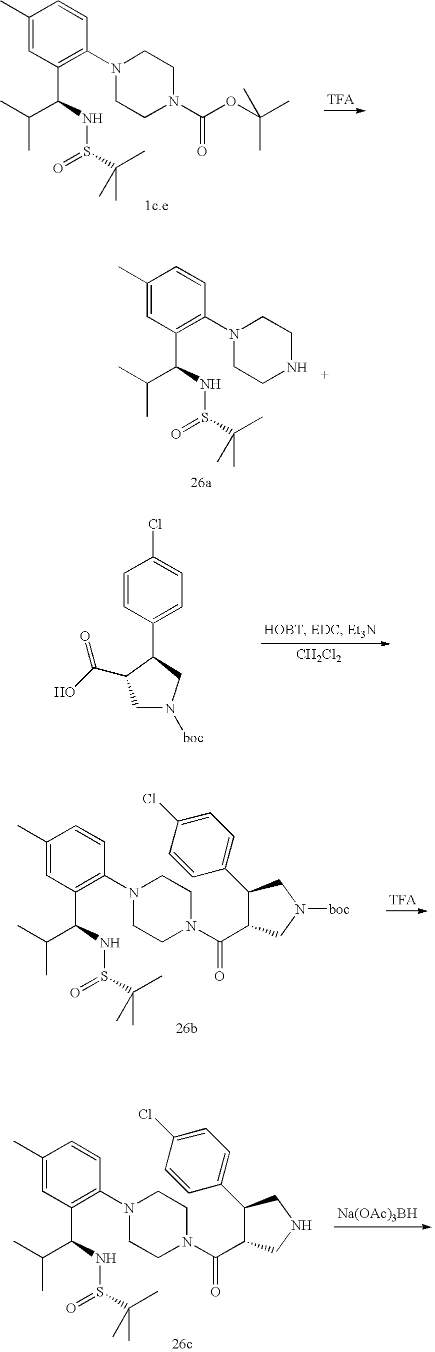

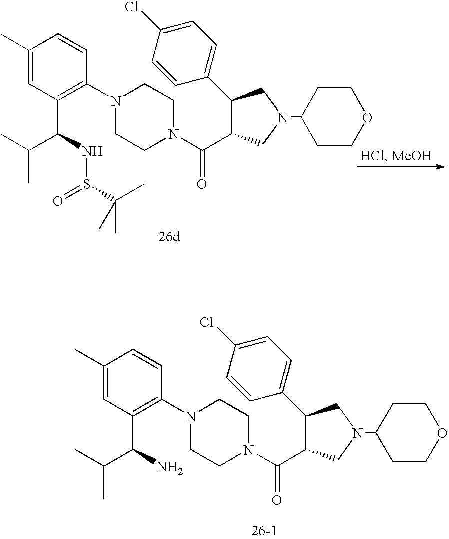

### Step 26A: Compound 26a

A stirring solution of 2-[4-(tert-butoxycarbonyl)-1-piperazinyl]-1-[1S-(S-t-butanesulfinamido)-2-methylpropyl]-5-methylbenzene 1c.e (2.71 g, 6.00 mmol) in CH₂Cl₂ (60 mL) was treated with TFA (12 mL) at room temperature for 40 min. The reaction mixture was carefully poured onto 0.1 N aqueous NaOH (200 mL) and extracted with CH₂Cl₂. The organics were dried over anhydrous MgSO₄, filtered and concentrated in vacuo to give the 26a as a yellow foam, which was used without further purification in the next step.

### Step 26B: Compound 26b

To a stirred solution of 4-(4-chlorophenyl)-pyrrolidine-1,3-dicarboxylic acid 1-tert-butyl ester (1.95 g, 6.00 mmol) and triethylamine (3.4 mL, 24.00 mmol) in CH₂Cl₂ (30 mL), HOBT (1.22 g, 9.00 mmol) was added under an atmosphere of N₂. After 30 min., the amine 26a, obtained in the previous step, was dissolved in CH₂Cl₂ (5 mL) and added to the mixture, followed by EDC (1.50 g, 2.60 mmol). The resulting solution was allowed to stir overnight. The reaction was quenched with 0.1 N HCl (100 mL) and extracted with CH₂Cl₂. The organics were separated, washed with saturated aqueous NaHCO₃ (50 mL) and brine. Drying (MgSO₄) and evaporation yielded a tan foam which was purified by column chromatography on silica gel, eluting with a 1:1 v/v mixture of hexanes and ethyl acetate to give 26b as a white foam,. Yield=2.36 g (3.59 mmol, 60% ).

### Step 26C: Compound 26c

3-(4-Chlorophenyl)-4-(4-{4-methyl-2-[(S)-2-methyl-1-((S)-2-methylpropane-2-sulfinylamino)propyl]phenyl}piperazine-1-carbonyl)-pyrrolidine-1-carboxylic acid tert-butyl ester 26b (1.97 g, 3.00 mmol) was dissolved in CH₂Cl₂ (30 mL) and treated with TFA (6 mL) for 1 h at room temperature. The reaction mixture was carefully poured onto aqueous 1N NaOH (200 mL) and extracted with CH₂Cl₂. The organics were dried over anhydrous MgSO₄, filtered and concentrated in vacuo to give 26c as a yellow foam, which was used without further purification in the next step.

### Step 26D: Compound 26d

A solution containing 2-methyl-propane-2-sulfinic acid [(S)-1-(2-{4-[4-(4-chloro-phenyl)-pyrrolidine-3-carbonyl]-piperazin-1-yl}-5-methyl-phenyl)-2-methylpropyl]-amide 26c (60 mg, 108 μmol) and 1,2-dichloroethane (1 mL) was treated with tetrahydro-4H-pyran-2-one (22 mg, 220 μmol). The mixture was shaken at room temperature for 1 h and then treated with Na(OAc)₃BH (46 mg, 217 μmol). The resulting heterogeneous mixture was shaken overnight. The reaction was quenched with saturated aqueous NaHCO₃ (3 mL) and extracted with CH₂Cl₂ (10 mL). The organic layer was separated, dried over anhydrous MgSO₄, filtered and evaporated to give 26d which was used in the next step without any further purification.

### Step 26E: Compound 26-1

The compound 26d from Step 26D was dissolved in MeOH (1 mL) and treated with HCl (65 μL of a 2 N solution in Et₂O). After 1 h, the volatiles were removed under a flow of N₂. The crude compound was dissolved in MeOH (1 mL) and purified by preparative HPLC/MS, to give the compound 26-1 as the TFA salt (30 mg, 39 μmol, 36% over the last two steps). LRMS m/z 539 (MH⁺).

### Example 27

### [(3R,4S)-4-(4-CHLORO-PHENYL)-1-(TETRAHYDRO-PYRAN-4-YL)-PYRROLIDIN-3-YL]-{4-[2-((S)-1-DIMETHYLAMINO-2-METHYL-PROPYL)-4-METHYL-PHENYL]-PIPERAZIN-1-YL}-METHANONE

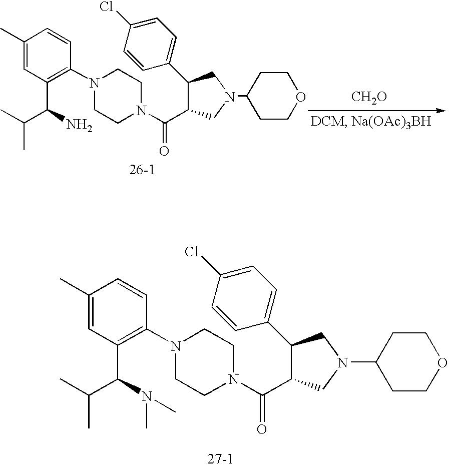

### Step 27A: Compound 27-1

{4-[2-((S)-1-Amino-2-methyl-propyl)-4-methyl-phenyl]-piperazin-1-yl}-[4-(4-chlorophenyl)-1-(tetrahydro-pyran-4-yl)-pyrrolidin-3-yl]-methanone 27-1 (10 mg, 13 μmol) was dissolved in CH₂Cl₂ (1 mL) and treated with aqueous formaldehyde (3 drops). Na(OAc)₃BH (30 mg, 142 μmol) was added and the mixture was stirred at room temperature for 2 h. The volatiles were removed under a N₂ stream and the residue was dissolved in MeOH (1 mL) and purified by preparative HPLC/MS to give compound 27-1. Yield=5.3 mg (6.7 μmol, 51% ). LRMS m/z 567.1 (MH⁺).

By the above procedures, the compounds of the following Table 27 were prepared.

TABLE 27

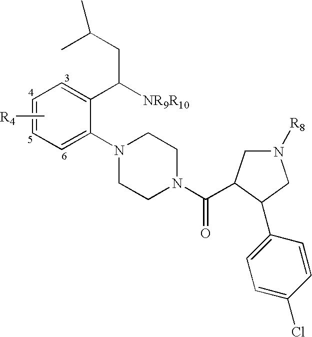

| Cpd | R₄ | NR₉R₁₀ | R₈ | (MH⁺) | MW |
|---|---|---|---|---|---|
| 27-1 | 4-Me | Me₂N | tetrahydro-4-pyranyl | 567 | 567.2 |
| 27-2 | 4-CF₃ | MeNH | iPr | 551 | 551.1 |
| 27-3 | 6-F | 3-piperidinylNH | iPr | 584 | 584.2 |
| 27-4 | 4-Me | Me₂N | 2-methyltetrahydro-3-furanyl | 567 | 567.2 |
| 27-5 | 4-Me | Me₂N | MeOCH₂CH(Me) | 555 | 555.2 |
| 27-6 | 4-Me | Me₂N | (MeOCH₂)₂CH | 585 | 585.2 |
| 27-7 | 4-Me | Me₂N | 2-methoxycyclohexyl | 595 | 595.3 |
| 27-8 | 4-Me | Me₂N | 2,2,5,5-tetramethyl-3- | 609 | 609.3 |
| | | | terahydrofuranyl | | |
| 27-9 | 4-Me | Me₂N | cyclohexyl | 565 | 565.2 |
| 27-10 | 4-Me | Me₂N | 1-ethyl-4-piperidinyl | 594 | 594.3 |
| 27-11 | 4-Me | Me₂N | 1-isobutyl-4-piperidinyl | 622 | 622.3 |
| 27-12 | 4-Me | Me₂N | 1-isopropy-4-piperidinyl | 608 | 608.3 |
| 27-13 | 4-Me | Me₂N | 1-acetyl-4-piperidinyl | 608 | 608.3 |
| 27-14 | 4-Me | Me₂N | iPr | 525 | 525.2 |

### Example 28

### N-[(S)-1-(2-{4-[(3R,4S)-4-(4-CHLORO-PHENYL)-1-(TETRAHYDRO-PYRAN-4-YL)-PYRROLIDINE-3-CARBONYL]-PIPERAZIN-1-YL}-5-METHYL-PHENYL)-2-METHYL-PROPYL]-3-DIMETHYLAMINO-PROPIONAMIDE

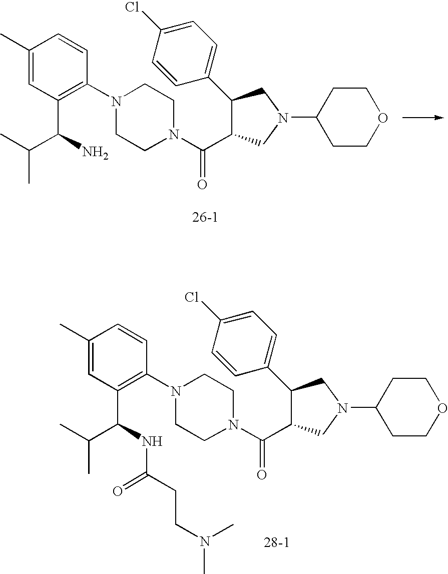

### Step 28A: Compound 28-1

{4-[2-((S)-1-Amino-2-methyl-propyl)-4-methyl-phenyl]-piperazin-1-yl}-[4-(4-chloro-phenyl)-1-(tetrahydro-pyran-4-yl)-pyrrolidin-3-yl]-methanone 26-1 (50 mg, 93 μmol) was dissolved in CH₂Cl₂ (1 mL) and treated with Hüinigs base (35 μL, 200 μmol), HOBT (19 mg, 140 μmol) and N,N-dimethyl-β-alanine hydrochloride (17 mg, 110 μmol). The resulting mixture was stirred at room temperature for 30 min. and then treated with EDC (27 mg, 140 μmol). The reaction was stirred overnight and then concentrated in vacuo. The crude residue was dissolved in MeOH (1 mL) and purified by preparative HPLC/MS to give 28-1. Yield=11.30 mg (17.7 μmol, 19% ). LRMS m/z 638 (MH⁺).

### Example 29

### 1-[(3R,4S)-3-{4-[2-((S)-1-AMINO-2-METHYL-PROPYL)-4-METHYL-PHENYL]-PIPERAZINE-1-CARBONYL}-4-(4-CHLORO-PHENYL)-PYRROLIDIN-1-YL]-PROPAN-1-ONE

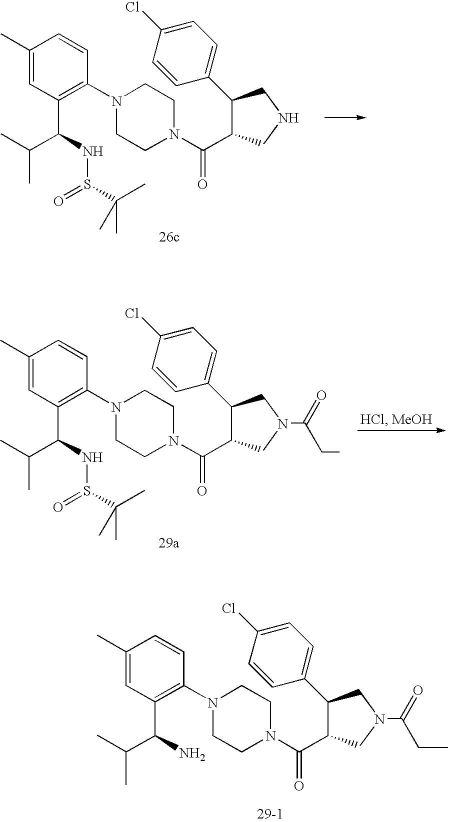

### Step 29A: Compound 29a

A solution containing 2-methyl-propane-2-sulfinic acid [(S)-1-(2-{4-[4-(4-chloro-phenyl)-pyrrolidine-3-carbonyl]-piperazin-1-yl}-5-methyl-phenyl)-2-methyl-propyl]-amide 26c (60 mg, 108 μmol), CH₂Cl₂ (1 mL) and Hünigs base (38 μL, 216 μmol) was treated with propionyl chloride (11 mg, 120 μmol). The resulting mixture was shaken at room temperature overnight. The reaction was concentrated under a flow of N₂ to give compound 29a which was used in the next step without further purification.

### Step 29B: Compound 29-1

The crude compound 29a above was dissolved in MeOH (1 mL) and treated with HCl (50 μL of a 4 M solution in dioxane). After 1 h, the volatiles were removed under a flow of N₂. The residue was dissolved in MeOH (1 mL) and purified by preparative HPLC/MS to give compound 29-1 (4 mg, 7.8 μmol, 7% over the last two steps). LRMS m/z 511 (MH⁺).

### Example 30

### N-[(S)-1-(2-{4-[(4S,5R)-5-(4-CHLORO-PHENYL)-2,2-DIMETHYL-[1,3]DIOXOLANE-4-CARBONYL]-PIPERAZIN-1-YL}-3-FLUORO-PHENYL)-2-METHYL-PROPYL]-3-DIMETHYLAMINO-PROPIONAMIDE

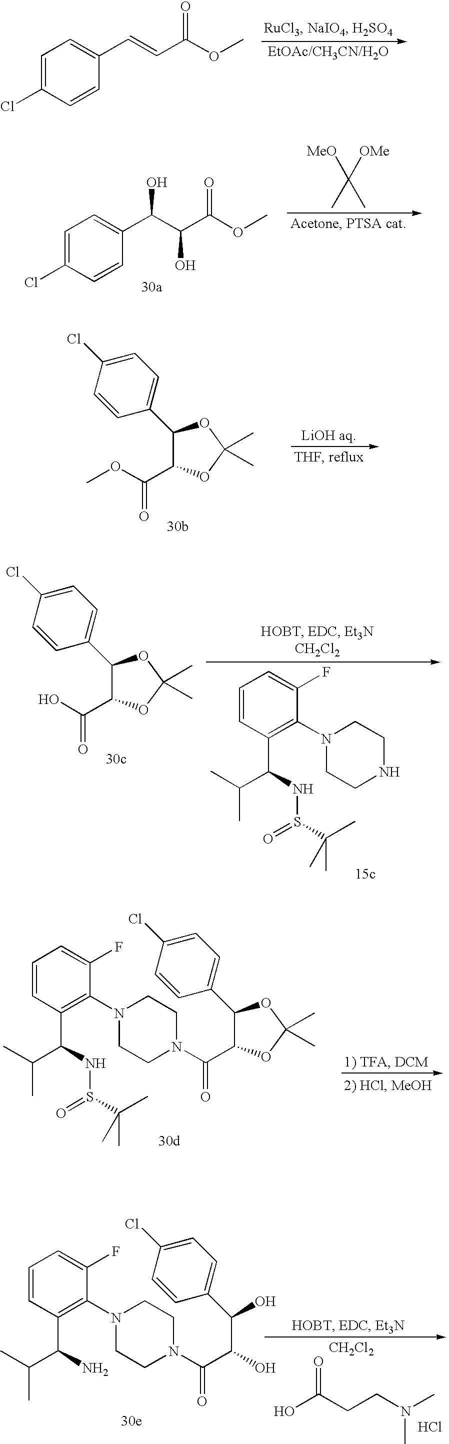

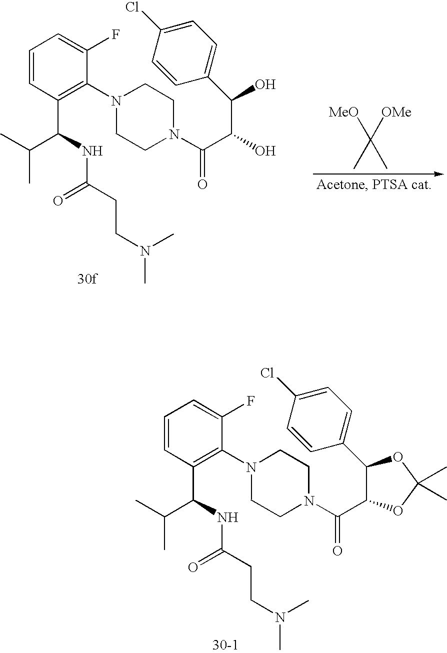

### Step 30A: Compound 30a

To a stirring suspension of sodium periodate (642 mg, 3.0 mmol) in H₂O (1.5 mL) was added 2 N H₂SO₄ (400 μL, 0.4 mmol). After ˜10 min. almost all solids had dissolved. The reaction was cooled to 0° C. (ice/water bath) and RuCl₃ (100 μL of a 0.1 M aqueous solution, 0.01 mmol) was added. After 10 min., EtOAc (6 mL), MeCN (6 mL) and (E)-3-(4-chlorophenyl)acrylic acid methyl ester (393 mg, 2.0 mmol) were sequentially added. The mixture was allowed to slowly reach room temperature over 4 h. Water and ethyl acetate were added. The organic layer was separated, washed with brine, dried and concentrated. The resulting oil was triturated with hexanes to give 30a as crystals which grew over 2 days. Yield=140 mg (0.61 mmol, 30% ).

### Step 30B: Compound 30b

3-(4-Chlorophenyl)-2,3-dihydroxy-propionic acid methyl ester 30a (135 mg, 0.59 mmol) was dissolved in acetone (1.2 mL) and treated with 2,2-dimethoxypropane (0.45 mL) and a catalytic amount of p-toluenesulfonic acid monohydrate (3 mg). The resulting mixture was stirred at room temperature for 20 h. The volatiles were removed in vacuo and the resulting crude material was used without further purification in the next step.

### Step 30C: Compound 30c

LiOH (3 mL of a 1 N aqueous solution) was added to a solution containing 5-(4-chlorophenyl)-2,2-dimethyl-[1,3]dioxolane-4-carboxylic acid methyl ester 30b (158 mg, 0.59 mmol) in THF (3 mL). The resulting mixture was stirred under reflux for 1.5 h. After cooling to room temperature, the mixture was diluted with EtOAc and washed with 0.2 N HCl and brine. The organics were dried over anhydrous MgSO₄, filtered and evaporated in vacuo to yield 30c as a yellow oil (180 mg).

### Step 30D: Compound 30d

HOBT (117 mg, 0.87 mmol) was added to a stirring mixture containing 5-(4-chloro-phenyl)-2,2-dimethyl-[1,3]dioxolane-4-carboxylic acid 30c (150 mg, 0.58 mmol) and triethylamine (330 μL, 2.32 mmol) in CH₂Cl₂ (3 mL). After 20 min., EDC (145 mg, 0.75 mmol) was added under N₂, and the resulting solution was stirred for another 30 min. 2-Methyl-propane-2-sulfinic acid [(S)-1-(3-fluoro-2-piperazin-1-yl-phenyl)-2-methyl-propyl]-amide 15c (206 mg, 0.58 mmol) in CH₂Cl₂ (2 mL) was introduced and the resulting mixture was stirred at room temperature for 20 h. The reaction was quenched with 0.1 N HCl (100 mL) and extracted with CH₂Cl₂. The organics were separated, washed with saturated aqueous NaHCO₃ (50 mL) and brine. Drying (MgSO₄) and evaporation gave 30d as a white foam, which was used in the next step without further purification.

### Step 30E: Compound 30e

2-Methyl-propane-2-sulfinic acid [(S)-1-(2-{4-[5-(4-chloro-phenyl)-2,2-dimethyl-[1,3]dioxolane-4-carbonyl]-piperazin-1-yl}-3-fluoro-phenyl)-2-methyl-propyl]-amide 30d (347 mg, 0.58 mmol) was dissolved in CH₂Cl₂ (3 mL) and treated with TFA (3 mL). The resulting mixture was stirred at room temperature for 1 h and then concentrated under reduced pressure. The residue was taken up in EtOAc (10 mL) and washed with saturated aqueous NaHCO₃ (30 mL). The organic layer was separated, dried over anhydrous MgSO₄, filtered and evaporated. The crude material was dissolved in MeOH (3 mL) and treated with HCl (500 μL of a 2 N solution in Et₂O) for 1.5 h. Concentration under vacuum, followed by purification by 2 preparative TLC plates (thickness—500 μm), eluting with a 400:50:2 v/v mixture of CHCl₃: MeOH: NH₄OH respectively, gave compound 30e as a colorless film (44 mg, 98 μmol, 17% ). LRMS m/z 450.1 (MH⁺).

### Step 30F: Compound 30f

HOBT (16 mg, 0.12 mmol) was added to a stirring mixture containing 1-{4-[2-((S)-1-amino-2-methyl-propyl)-6-fluoro-phenyl]-piperazin-1-yl}-3-(4-chloro-phenyl)-2,3-dihydroxy-propan-1-one 30e (35 mg, 78 μmol), dimethyl-β-alanine hydrochloride (13 mg, 80 μmol) and triethylamine (44 μL, 0.31 mmol) in CH₂Cl₂ (1 mL). After 30 min., EDC (23 mg, 0.12 mmol) was added under N₂, and the resulting solution was stirred for 48 h. Constant monitoring by LCMS led to the addition of extra dimethyl-β-alanine hydrochloride, HOBT and EDC. At the end of the reaction, three dimethyl-β-alanine units had been incorporated onto the molecule, presumably forming the desired amide, plus two esters. The reaction was worked up and the residue was treated with THF/LiOH aq. for 2 h. at room temperature. LCMS now shows the desired compound. The reaction was worked up and purified by preparative TLC plate (thickness—500 μm), eluting with a 400:50:2 v/v mixture of CHCl₃: MeOH: NH₄OH, respectively. Compound 30f was obtained as a colorless film (20 mg, 36 μmol, 46% ). LRMS m/z 549 (MH⁺).

### Step 30G: Compound 30-1

N-[(S)-1-(2-{4-[3-(4-Chloro-phenyl)-2,3-dihydroxy-propionyl]-piperazin-1-yl}-3-fluoro-phenyl)-2-methyl-propyl]-3-dimethylamino-propionamide 30f (10 mg, 18 μmol) was dissolved in acetone (1 mL) and treated with 1,2-dimethoxypropane (200 μL) and a catalytic amount of p-toluenesulfonic acid monohydrate (3 mg). The resulting mixture was stirred at room temperature overnight. The volatiles were removed in vacuo and the resulting material was purified by preparative TLC plate (thickness—500 μm), eluting with a 400:50:2 v/v mixture of CHCl₃: MeOH: NH₄OH, respectively to give compound 30-1. Yield=3.5 mg (6 μmol, 33% ). LRMS m/z 589 (MH⁺).

### Example 31

### {4-[2-((S)-1-AMINO-3-METHYL-BUTYL)-4-TRIFLUOROMETHYL-PHENYL]-PIPERAZIN-1-YL}-[(3R,4S)-1-BENZYL-4-(2,4-DICHLORO-PHENYL)-PYRROLIDIN-3-YL]-METHANONE

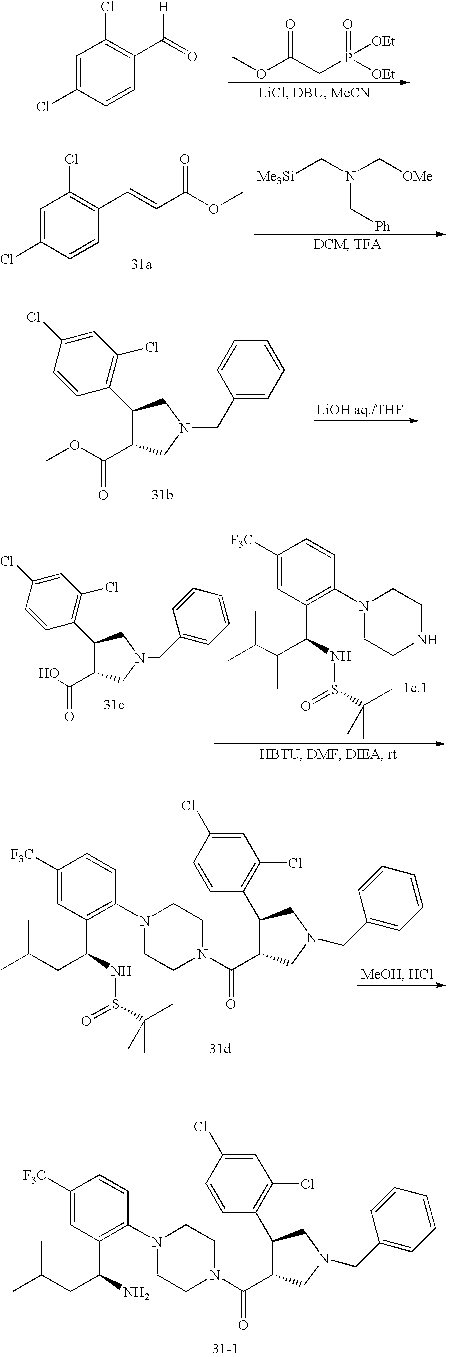

### Step31A: Compound 31a

To a stirring suspension of LiCl (2.54 g, 60.0 mmol) in MeCN (415 mL), methyl diethylphosphonoacetate (11.0 mL, 60.0 mmol), DBU (9.0 mL, 60.0 mmol) and 2,4-dichlorobenzaldehyde (8.75 g, 50.0 mmol) were added sequentially. The initial suspension turned into a solution and then to a milky suspension in ˜30 min. The mixture was stirred at room temperature for 18 h. then was diluted with Et₂O (300 mL), washed with 0.1 N HCl and brine. The organics were dried over anhydrous MgSO₄, filtered and evaporated under reduced pressure to yield an oily residue. This was dissolved in hot MeOH (250 mL), and crystallized to give 31a as a white solid. Yield=8.18 g (35.4 mmol, 71% ).

### Step 31B: Compound 31b

TFA (156 μL, 2.1 mmol) was added dropwise to a stirring solution containing (E)-3-(2,4-dichlorophenyl)-acrylic acid methyl ester 31a (4.85 g, 21.0 mmol) and benzyl-methoxymethyl-trimethylsilanylmethyl-amine (5.37 mL,21.0 mmol) in CH₂Cl₂ (84 mL). The mixture was stirred at room temperature for 18 h. LCMS indicated clean conversion to product. The reaction was placed in a separation funnel, washed twice with saturated aqueous NaHCO₃ (200 mL), dried over anhydrous MgSO₄, filtered and evaporated under reduced pressure to yield an oily residue. Purification was achieved by column chromatography, eluting with a 9:1 v/v mixture of hexanes and EtOAc, respectively. Compound 31b was isolated as an oil (4.49 g, 12.3 mmol, 59% ).

### Step 31C: Compound 31c

LiOH (25 mL of a 1 N aqueous solution) was added to a solution containing 1-benzyl-4-(2,4-dichloro-phenyl)-pyrrolidine-3-carboxylic acid methyl ester (31b) (1.82 g, 5.0 mmol) in THF (25 mL). The resulting mixture was stirred under reflux for 1 h, and the reaction progress was monitored by both TLC (3:1 hexanes/EtOAc) and LCMS. After cooling to room temperature, the volatiles were removed in vacuo to yield a white suspension, which was filtered and air-dried to yield 31c as a white solid (1.28 g, 3.6 mmol, 72% ).

### Step 31D: Compound 31d

HBTU (50 mg, 0.13 mmol) was added to a stirring suspension of 1-benzyl-4-(2,4-dichlorophenyl)-pyrrolidine-3-carboxylic acid 31c (35 mg, 0.10 mmol) and Hünigs base (35 μL, 0.20 mmol) in DMF (1 mL). A tan solution resulted, which was kept under N₂ for 20 min. 2-Methyl-propane-2-sulfinic acid [(S)-3-methyl-1-(2-piperazin-1-yl-5-trifluoromethyl-phenyl)-butyl]-amide 1c.1 (42 mg, 0.10 mmol) in DMF (0.5 mL) was introduced via syringe, and the resulting mixture allowed to stir at room temperature for 2 h. The reaction was deemed complete by LCMS after 2 h. The reaction mixture was diluted with ethyl acetate, washed with NaHCO₃ solution and brine, dried and evaporated to give 31d, which was used in the next step without further purification.

### Step 31E: Compound 31-1

2-Methyl-propane-2-sulfinic acid [(S)-1-(2-{4-[1-benzyl-4-(2,4-dichloro-phenyl)-pyrrolidine-3-carbonyl]-piperazin-1-yl}-5-trifluoromethyl-phenyl)-3-methyl-butyl]-amide 31d (75 mg,0.10 mmol) was dissolved in MeOH (1 mL) and treated with HCl (80μL of a 2 N solution in Et₂O, 0.15 mmol) for 1 h at room temperature. The volatiles were removed in vacuo and the residue was purified by preparative TLC plate (thickness—500 μm), eluting with a 400:50:2 v/v mixture of CHCl₃: MeOH: NH₄OH, respectively. Compound 31-1 was isolated as a colorless film. Yield=34 mg (54 μmol, 54% ). LRMS m/z 647 (MH⁺).

### Example 32

### N-[(S)-1-(2-{4-[(3R,4S)-4-(2,4-DICHLORO-PHENYL)-1-ISOPROPYL-PYRROLIDINE-3-CARBONYL]-PIPERAZIN-1-YL}-3-FLUORO-PHENYL)-2-METHYL-PROPYL]-3-DIMETHYLAMINO-PROPIONAMIDE

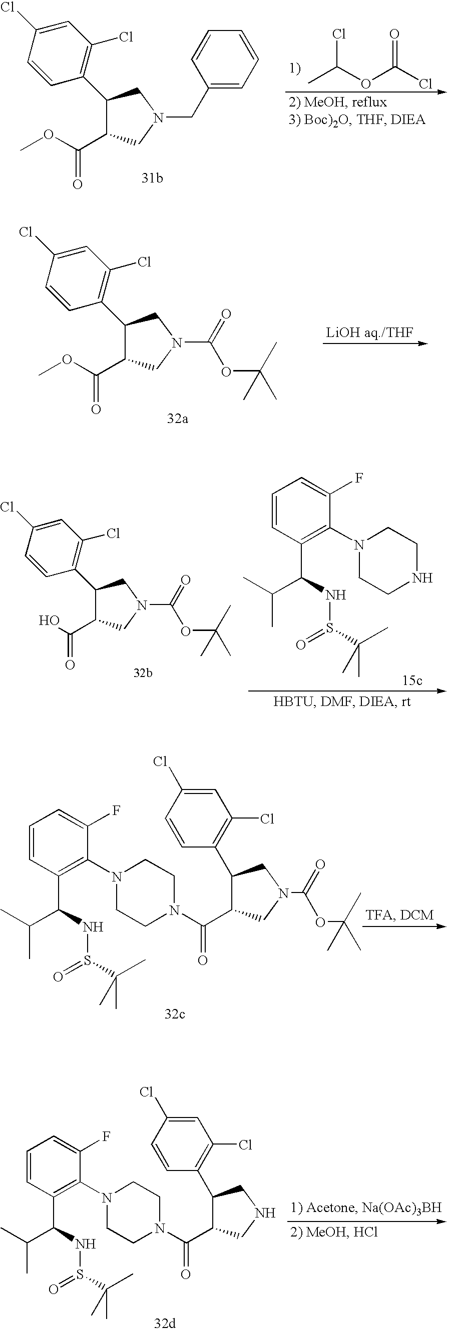

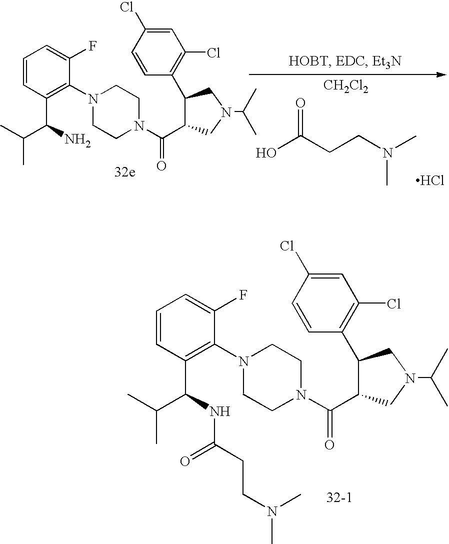

### Step 32A: Compound 32a

To a 0° C. solution of 1-benzyl-4-(2,4-dichlorophenyl)-pyrrolidine-3-carboxylic acid methyl ester 31b (1.09 g, 3.0 mmol) in 1,2-dichloroethane (15 mL), 1-chloroethyl chloroformate (515 mg, 3.6 mmol) was added dropwise under N₂. After 15 min. at 0° C., the mixture was slowly warmed to room temperature, and then to reflux. Reflux was maintained for 3 h, after which time LCMS indicated the formation of product. The reaction was cooled to room temperature, the volatiles were removed in vacuo and MeOH (30 mL) was introduced. The mixture was refluxed for an additional 2 h. The solvent removed under reduced pressure. The crude residue was taken up in THF (30 mL), treated with Hünigs base (1.0 mL, 6.0 mmol) and Boc anhydride (720 mg, 3.3 mmol). The resulting mixture was stirred at room temperature for 5 h. Following workup and concentration, the residue was purified by column chromatography on silica gel, eluting with a gradient of 9:1 to 4:1 v/v mixture of hexanes and EtOAc, to give 32a (805 mg, 2.2 mmol, 73% ).

### Step 32B: Compound 32b

LiOH (10 mL of a 1 N aqueous solution) was added to a solution containing 4-(2,4-dichlorophenyl)-pyrrolidine-1,3-dicarboxylic acid 1-tert-butyl ester 3-methyl ester 32a (805 mg, 2.15 mmol) in THF (10 mL). The resulting mixture was stirred under reflux for 1 h. After cooling to room temperature, the reaction was acidified to pH ˜1 with 0.1 N HCl and extracted with EtOAc. The organics were washed with brine, dried over anhydrous MgSO₄, filtered and evaporated under reduced pressure to yield 32b as a white solid, which was used in the next step as is. Yield=758 mg (2.11 mmol, 98% ).

### Step 32C: Compound 32c

HBTU (493 mg, 1.3 mmol) was added to a stirring solution of 4-(2,4-dichloro-phenyl)-pyrrolidine-1,3-dicarboxylic acid 1-tert-butyl ester 32b (360 mg, 1.0 mmol) and Hünigs base (350 μL, 2.0 mmol) in DMF (10 mL). A tan solution resulted, which was kept under N₂ for 20 min. 2-Methyl-propane-2-sulfinic acid [(S)-1-(3-fluoro-2-piperazin-1-yl-phenyl)-2-methyl-propyl]-amide 15c (355 mg, 1.0 mmol) in DMF (5 mL) was introduced via syringe, and the resulting mixture allowed to stirr at room temperature for 16 h. Work up gave a residue that was purified by column chromatography on silica gel, eluting with a 1:1 v/v mixture of hexanes and EtOAc to give 32c. Yield=515 mg (0.74 mmol, 74% ).

### Step 32D: Compound 32d

TFA (1.5 mL) was added to a stirring solution of 3-(2,4-dichloro-phenyl)-4-(4-{2-fluoro-6-[(S)-2-methyl-1-((S)-2-methyl-propane-2-sulfinylamino)-propyl]-phenyl}-piperazine-1-carbonyl)-pyrrolidine-1-carboxylic acid tert-butyl ester 32c (515 mg, 0.74 mmol) in CH₂Cl₂ (7.5 mL). After 1 h., the reaction was carefully poured onto saturated aqueous NaHCO₃ (100 mL). The organic layer was separated, washed with saturated aqueous NaHCO₃ (50 mL) and brine (50 mL), dried over anhydrous MgSO₄ and filtered. Evaporation gave 32d as a beige foam, which was used in the next step without further purification.

### Step 32E: Compound 32e

2-Methyl-propane-2-sulfinic acid [(S)-1-(2-{4-[4-(2,4-dichloro-phenyl)-pyrrolidine-3-carbonyl]-piperazin-1-yl}-3-fluoro-phenyl)-2-methyl-propyl]-amide 32d obtained in the previous step (290 mg, 0.49 mmol) was dissolved in CH₂Cl₂ (2.5 mL) and treated with acetone (2.5 mL) and Na(OAc)₃BH (412 mg, 1.94 mmol). After 18 h. at room temperature, LCMS indicated the reaction was complete. Methylene chloride was added and the mixture was washed with sat. NaHCO₃ and brine. The organic layer was dried and evaporated to a residue which was dissolved in MeOH (5 mL) and treated with HCl (370 μL of a 2 N solution in Et₂O) for 1 h. The volatiles were removed in vacuo and the crude amine 32e was used without any further purification in the next step.

### Step 32F: Compound 32-1

HOBT (22 mg, 160 μmol) was added to a stirring mixture containing {4-[2-((S)-1-amino-2-methyl-propyl)-6-fluoro-phenyl]-piperazin-1-yl}-[4-(2,4-dichloro-phenyl)-1-isopropyl-pyrrolidin-3-yl]-methanone hydrochloride 32e (58 mg, 107 μmol), dimethyl-β-alanine hydrochloride (17 mg, 110 μmol) and Hünigs base (75 μL, 428 μmol) in CH₂Cl₂ (1.1 mL). After 30 min., EDC (31 mg, 160 μmol) was added under N₂, and the resulting solution was stirred overnight. The reaction was concentrated under N₂, and the residue was purified by preparative HPLC/MS to give 32-1. Yield=37.6 mg (43.5 μmol, 41% ). LRMS m/z 634 (MH⁺).

### Example 33

### (3R,4S)-4-(4-DIMETHYLAMINO-PHENYL)-1-ISOPROPYL-PYRROLIDINE-3-CARBOXYLIC ACID METHYL ESTER

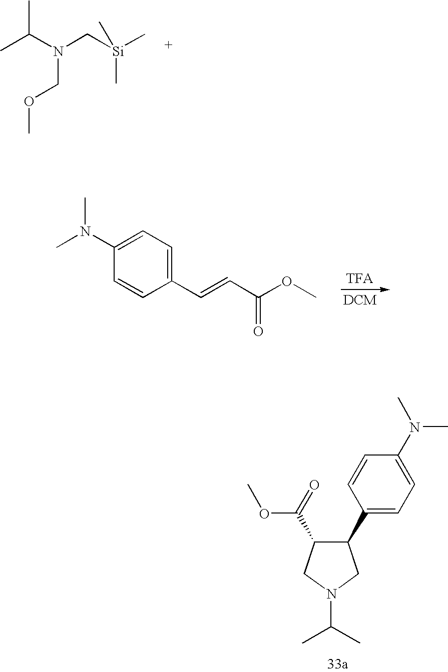

### Step 33A: Synthesis of trans-1-isopropyl-3-carboxymethyl-4-(4′dimethylaminophenyl)-pyrrolidine 33a

A mixture of 2 mmol (411 mg) of methyl 4-dimethylaminocinnamate and 200 μL trifluoroacetic acid in 2 mL of dichloromethane was cooled to 0° C. and with vigorous stirring, 758 mg (4 mmol) of isopropylmethoxymethyltrimethylsilylmethylamine in 2 mL of dichloromethane was added dropwise. The mixture was stirred for 4 hours at room temperature. The reaction mixture was washed with water and the organic layer was dried and evaporated to give a residue which was purified on silica (dichloromethane/methanol 19:1) to give 33a (320 mg, 55% ). The isopropylmethoxymethyltrimethylsilylmethylamine was synthesized as follows: isopropylamine (29.56 g, 0.5 mole) and trimethylchloromethylsilane (30.67 g, 0.25 mole) were heated for 16 hours to 60° C. in a sealed flask. Excess reagents were removed in vacuo to give isopropyltrimethylsilylmethylamine (>95% pure, 26.7 g, 73.5% ). To 37% fornmaldehyde in water (12.5 g, 0.154 mole), cooled to 0° C., isopropyltrimethylsilylmethylamine (16 g, 0.11 mole) was added dropwise and stirred 10 additional minutes at room temperature. Methanol (12.5 mL) was added and the mixture saturated with solid potassium carbonate. After stirring for one hour, the organic layer was separated, saturated with solid potassium carbonate and stirred for 48 hours. The reaction mixture was filtered and excess of solvents removed in vacuo. Isopropylmethoxymethyl-trimethylsilylmethylamine (>95% pure, 13 g, 62.4% ) was recovered.

Compound 14a (0.1 mmol, 35 mg) was dissolved in 0.5 mL of dioxane and 0.2 mmol of trimethylaluminum solution in toluene (0.1 mL) was added dropwise. The mixture was stirred for 30 minutes at room temperature and then compound 33a (0.1 mmol, 29 mg) in 0.2 mL of dioxane was added dropwise. The mixture was stirred for 30 minutes at room temperature and for 2 hours at 80° C. The mixture was cooled, quenched with 2 M hydrochloric acid, extracted with ethyl acetate, dried, concentrated in vacuo and purified by HPLC to give 34-1 (25.3 mg, 42% ).

### Example 34

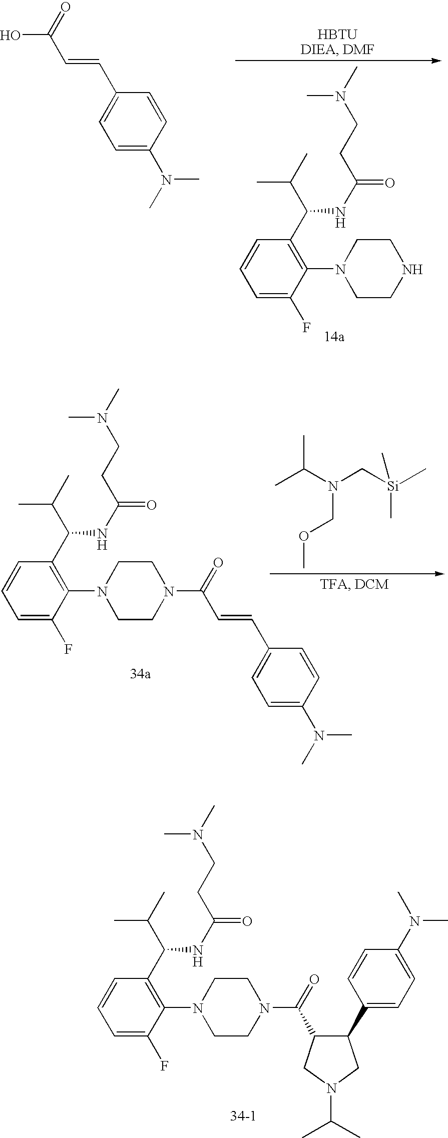

### Step 34A: Compound 34a

4-Dimethylaminocinnamic acid (96 mg, 0.5 mmol), HBTU (209 mg, 0.55 mmol), DIEA 0.2 mL and DMF (1 mL) were stirred for 15 minutes. Compound 14a (175 mg, 0.5 mmol) in 0.5 mL DMF was added dropwise and the mixture was stirred for 4 hours. The mixture was quenched with water, extracted with ethyl acetate, dried over anh. MgSO₄ and the solvents removed in vacuo. Purification on silica (hexane/ethylacetate 1:1) gave compound 34a (191 mg, 73% ).

### Step 34B: Compound 34-1

Compound 34a (52.4 mg, 0.1 mmol) and 0.15 mL of trifluoroacetic acid in 0.5 mL of dichloromethane were stirred at 0° C. for 10 minutes. Isopropylmethoxymethyltrimethylsilylmethylamine (38 mg, 0.2 mmol) in 200 μL dichloromethane was added dropwise and the mixture was stirred for 4 hours. The mixture was washed with 1 M hydrochloric acid, solvents were removed in vacuo to give a residue which was purified by HPLC to give 34-1 (23 mg 38% ).

### Example 35

### (4S,5R)-5-{4-[2-((S)-1-AMINO-3-METHYL-BUTYL)-4-METHYL-PHENYL]-PIPERAZINE-1-CARBONYL}-4-(4-CHLORO-PHENYL)-OXAZOLIDIN-2-ONE

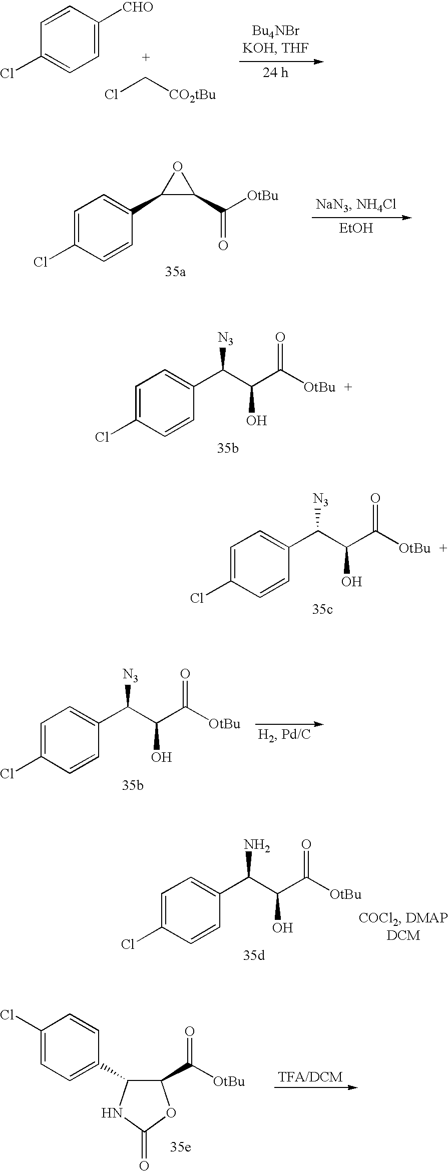

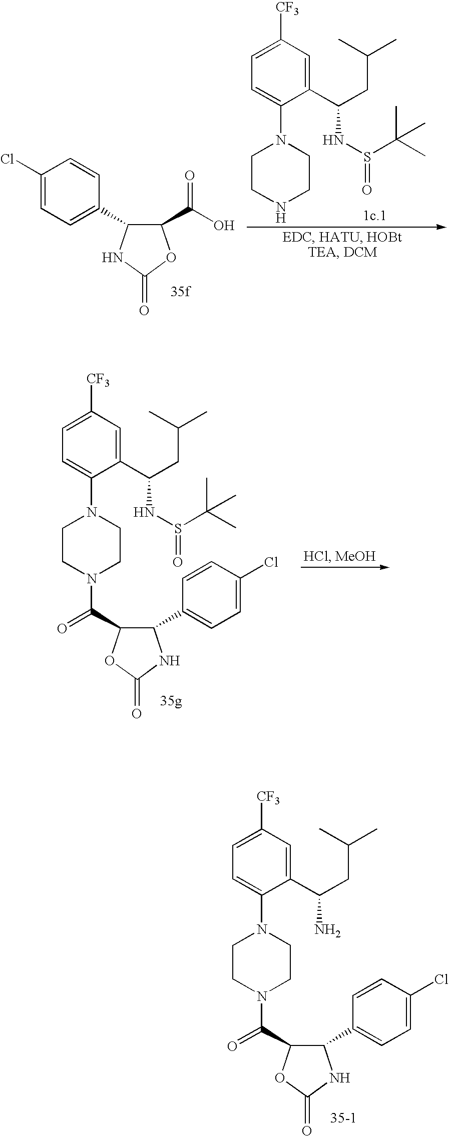

### Step 35A: Compound 35a

To the solution of 4-chlorobenzldehyde (5.00 g, 35.6 mmol) and t-butyl chloroacetate (0.11 mL, 42.7 mmol) in THF (107 mL) was added powered KOH (2.4 g, 42.7 mmol). Another 2.4 g of KOH was added after 5 h. The reaction was complete after 24 h. 100 mL H₂O was added and the mixture was extracted with EtOAc twice. The organic solution was dried over MgSO₄, filtered and concentrated. The product crystallized upon standing. It was further purified by column chromatography (Hex:EtOAc 9:1) to obtain 35a as white crystalline solid (4.82 g, 18.9 mmol) in 53% yield

### Step 35B: Compound 35b

To the solution of 35a (2.4 g, 9.42 mmol) in 52 mL EtOH was added NaN₃ (0.92 g, 14.13 mmol) and NH₄Cl (7.76 g, 14.14 mmol). The mixture was heated to reflux for 24 h. Another equivalent of NaN₃ (612 mg, 9.42 mmol) and NH4Cl (504 mg, 9.42 mmol) was added, and the reflux continued for 4 h. The reaction mixture was cooled, quenched with 100 mL H₂O and then 100 mL EtOAc was added. The aqueous layer was extracted with EtOAc again. Combined organic layers were washed with brine, dried over MgSO₄, filtered and concentrated. Purification by flash column chromatography afforded 1.914 g of 35b and 0.390 g minor product 35c. Total yield: 82%

### Step 35C: Compound 35d

To the solution of 35b (900 mg, 3.02 mmol) in 9 mL EtOAc was added 10% Pd/C (270 mg). The air in the reaction flask was removed and flushed with H₂ from a balloon. The procedure was repeated several times and the reaction was stirred at room temperature for 2 h. The reaction mixture was filtered through a pad of Celite® and concentrated to afford a white solid 35d (738 mg, 2.7 mmol) in 90% yield, including ca. 25% des-Cl by-product.

### Step 35D: Compound 35e

To a solution of 35d (810 mg, 2.99 mmol) and DMAP (732 mg, 5.98 mmol) in 30 mL CH₂Cl₂ was added COCl₂ (approx. 20% in toluene, 4.49 mmol) at 0° C. The solution turned yellow. The mixture warmed up to room temperature gradually and stirred for 16 h. The reaction mixture was quenched by adding saturated aqueous NaHCO₃, then was diluted with CH₂Cl₂. The organic layer was washed with 10% HCl(aq), dried over MgSO₄, filtered and concentratedto give 35e (1.4 g).

### Step 35E: Compound 35f

Compound 35e (1.4 g) was treated with TFA/DCM (8 mL each) at room temperature for 2 h and was concentrated to obtain 1.23 g of the acid 35f as white foam.

### Step 35F: Compound ³⁵ g

To the solution of 35f (530 mg, 2.19 mmol) and piperazine 1c.1 (727 mg, 1.74 mmol) in 7.3 mL CH₂Cl₂ was added EDC (HCl salt, 418 mg, 2.18 mmol), HOBt (294 mg, 2.18 mmol) and Et₃N (0.40 mL, 2.90 mmol). The reaction was stirred for 16 h. Another equivalent of EDC, HOBt and Et₃N was added. After 6 h, one equivalent of HATU was added. Reaction was stirred for another 20 h, and worked up by adding saturated NaHCO₃. The product was extracted by CH₂Cl₂ twice, dried over Na₂SO₄, filtered and concentrated. The residue was purified by flash column chromatography (2% MeOU/CH₂Cl₂) to give 35 g as a white foam (345 mg, 0.54 mmol).

### Step 35G: Compound 35-1

The sulfanimde 35 g (340 mg, 0.53 mmol) in 6 mL MeOH was treated with HCl (4.0 M in 1,4-dioxane, 0.27 mL) for 1 h. The solvent was removed in vacuo to give a yellow foam (360 mg). 20 mg of the foam was purified by HPLC to yield 35-1 as the TFA salt (10.3 mg, 0.016 mmol). LCMS 539 (MH⁺)

By the above procedures, the compounds of the following Table 35 were prepared.

TABLE 35

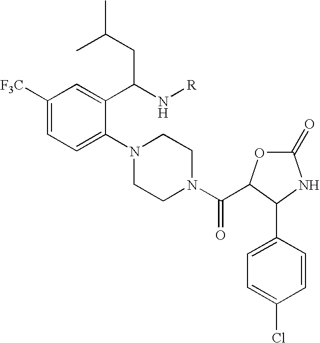

| Cpd | R | MW | MS |
|---|---|---|---|
| 35-1 | H | 539.0 | 539 |
| 35-2 | C(O)CH₂CH₂NH₂ | 609.1 | 610 |
| 35-3 | C(O)CH₂CH₂NHMe | 623.1 | 624 |
| 35-4 | C(O)CH₂CH₂NMe₂ | 637.1 | 638 |
| 35-5 | C(O)CH₂NHMe | 609.1 | 610 |
| 35-6 | C(O)CH(Me)NH₂ | 609.1 | 610 |

### Example 36

### {4-[2-((S)-1-AMINO-3-METHYL-BUTYL)-4-TRIFLUOROMETHYL-PHENYL]-PIPERAZIN-1-YL}-[(3R,4R)-4-(4-CHLORO-PHENYL)-TETRAHYDRO-THIOPHEN-3-YL]-METHANONE

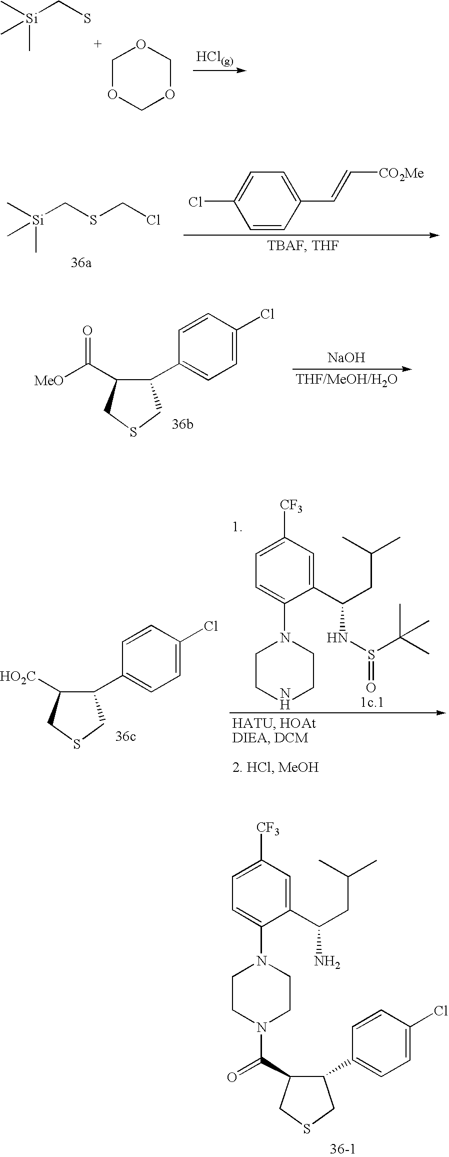

### Step 36A: Compound 36a

HCl was bubbled into a mixture of trimethylsilylmethyl sulfide (4.98 g, 41.4 mmol) and trioxane (1.28 g, 14.2 mmol) at −10° C. for 80 min. The reaction was maintained at 0° C. for 16 h and the aqueous layer was removed. CaCl₂ was added to the remaining oil and the mixture was stirred for 2 h. The crude oil was distilled under reduced pressure (˜10 mm Hg, b.p. 60° C.) to afford 36a as a colorless oil (3.70 g, 22.9 mmol) in 53% yield.

### Step 36B: Compound 36b

To a solution of 36a (1.00 g, 5.9 mmol) and cis-methyl 4-chlorocinnamate (900 mg, 4.6 mmol) in THF (23 mL) was added TBAF (1.0 M in THF, 6.9 mmol). Reaction was almost complete after 1 h by GC/MS, and was stirred for another 16 h. The reaction was quenched with H₂O, extracted with EtOAc, washed with 10% HCl twice and brine, dried over MgSO₄, filtered and concentrated to give 36b (1.192 g clear oil, 4.64 mmol) in quantitative yield.

### Step 36C: Compound 36c

Compound 36b (700 mg, 2.75 mmol) was dissolved in H₂O/THF/MeOH (14 mL, 14 mL, 10 mL) and NaOH (50% , 0.2 mL) was added to the solution. The reaction mixture was stirred for 2 h at room temperature and then concentrated at reduced pressure. The remaining solution was diluted with H₂O and extracted with Et₂O. The aqueous solution was acidified with 10% HCl then extracted with EtOAc twice to afford the acid 36c (625 mg, 2.58 mmol) in 96% yield after evaporation.

### Step 36D: Compound 36d

To the mixture of 36c (305 mg, 1.26 mmol) and piperazine 1c.1 (480 mg, 1.14 mmol) in CH₂Cl₂ was added HOBt (0.5 M in DMF, 3.1 mL), HATU (590 mg, 1.90 mmol) and DIEA (0.36 mL, 2.28 mmol). The reaction mixture was stirred at room temperature for 16 h, and then quenched with saturated NaHCO₃. The mixture was extracted with CH₂Cl₂, dried over Na₂SO₄, filtered, and concentrated. The two diastereomers were separate on TLC (Hex:EtOAc 9:1). After flash column chromatography (Hex:EtOAc 9:1 to 1:1), the mixture of two isomers 36d (319 mg, 0.50 mmol) was obtained in 43% yield.

### Step 36E: Compound 36e

The sulfanimde 36d in 5 mL MeOH was treated with HCl (4.0 M in 1,4-dioxane, 0.2 mL) for 30 min and the solvent was evaporated. One fifth of the product was purified by HPLC to afford the TFA salt of 36-1 (27.8 mg, 0.043 mmol) in 43% yield. LCMS 540 (MH⁺)

By the above procedures, the compounds of the following Table 36 were prepared.

TABLE 36

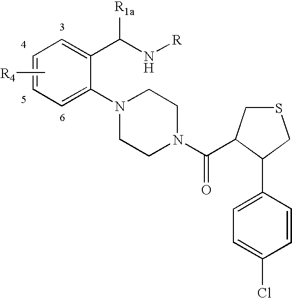

| Cpd | R₄ | R₁ₐ | R | Isomer | MW | MS |
|---|---|---|---|---|---|---|
| 36-1 | 4-CF₃ | i-Bu | H | — | 540.1 | 540 |
| 36-2 | 4-CF₃ | i-Bu | C(O)CH₂CH₂NMe₂ | — | 639.2 | 639 |
| 36-3 | 4-CF₃ | i-Bu | C(O)CH₂CH₂NHMe | — | 625.2 | 625 |
| 36-4 | 4-CF₃ | i-Bu | C(O)CH(Me)NH₂ | — | 611.2 | 611 |
| 36-5 | 4-CF₃ | i-Bu | C(O)CH₂NHMe | — | 611.2 | 611 |
| 36-6 | 6-F | i-Pr | C(O)CH₂CH₂NMe₂ | I | 575.2 | 575 |
| 36-7 | 6-F | i-Pr | C(O)CH₂CH₂NMe₂ | II | 575.2 | 575 |

### Example 37

### {4-[2-((S)-1-AMINO-3-METHYL-BUTYL)-4-TRIFLUOROMETHYL-PHENYL]-PIPERAZIN-1-YL}-[(3R,4R)-4-(4-CHLORO-PHENYL)-1,1-DIOXO-TETRAHYDRO-1LAMBDA*6*-THIOPHEN-3-YL]-METHANONE

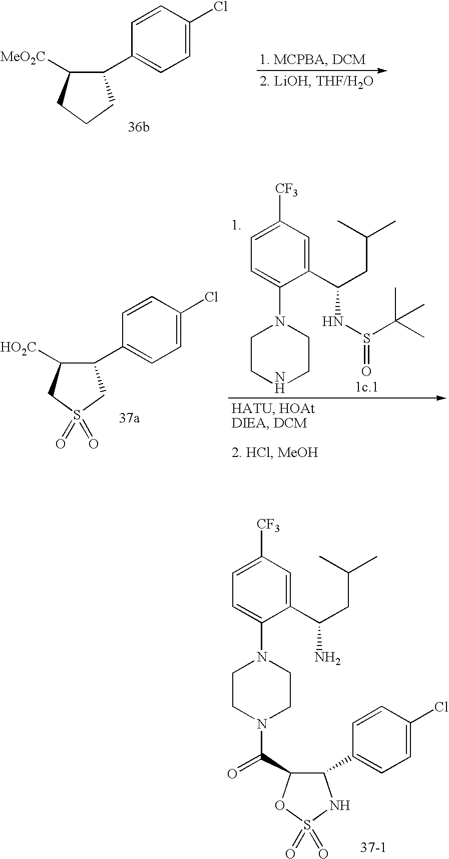

### Step 37A: Compound 37a

To a solution of 36b (589 mg, 2.3 mmol) in CH₂Cl₂ (15 mL) was added MCPBA (75% max, 782 mg, 3.4 mmol). The reaction mixture was stirred at room temperature for 2 h. then was diluted with EtOAc and washed with 5% NaHCO₃ twice. The organic layer was concentrated and the residue was purified by flash column chromatography (2% MeOH/CH₂Cl₂) to afford the sulfone methyl ester (166 mg, 0.58 mmol) in 25% yield. The sulfone methyl ester (166 mg, 0.58 mmol) was hydrolyzed by the same procedure as Step 36C to obtain the acid 37a.

### Step 37B Compound 37-1

To the mixture of 37a (assumed quantitative yield from previous step, 0.58 mmol) and piperazine 1c.1 (255 mg, 0.61 mmol) was added HOAt (0.5 M in DMF, 1.74 mL), HATU (330 mg, 0.87 mmol) and DIEA (0.20 mL, 1.16 mmol). The reaction mixture was stirred at room temperature for 16 h, and was quenched with saturated NaHCO₃. The mixture was extracted with CH₂Cl₂, dried over Na₂SO₄, filtered, and concentrated. Purification by flash column chromatography (2% MeOH/CH₂Cl₂) afforded the sulfanimide (330 mg, 0.49 mmol, 84% yield) which was dissolved in 5 mL MeOH. HCl (4.0 M in 1,4-dioxane, 0.25 mL) was added and the mixture was stirred for 30 min and the solvent was evaporated. 6% of the crude mixture (0.03 mL) was purified by HPLC to afford the TFA salt of 37-1 (8.5 mg, 0.012 mmol) in 40% yield. LCMS 572 (MH⁺)

By the above procedures, the compounds of the following Table 37 were prepared.

TABLE 37

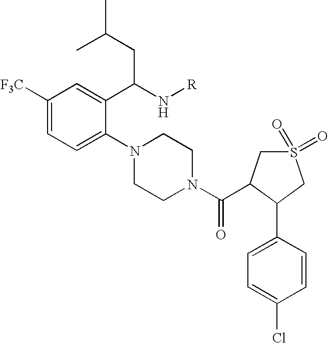

| Cpd | R | MW | MS |
|---|---|---|---|
| 37-1 | H | 572.1 | 572 |
| 37-2 | —C(O)CH₂CH₂NMe₂ | 671.2 | 671 |
| 37-3 | —C(O)CH₂CH₂NHMe | 657.2 | 657 |
| 37-4 | —C(O)CH₂NHMe | 643.2 | 643 |
| 37-5 | —C(O)CH(Me)NH₂ | 643.2 | 643 |
| 37-6 | —CH₂CH₂NHMe | 629.2 | 629 |

### Example 38

### {4-[2-((S)-1-AMINO-3-METHYL-BUTYL)-4-TRIFLUOROMETHYL-PHENYL]-PIPERAZIN-1-YL}-[(3R,4R)-4-(4-CHLORO-PHENYL)-1-OXO-TETRAHYDRO-1LAMBDA*4*-THIOPHEN-3-YL]-METHANONE

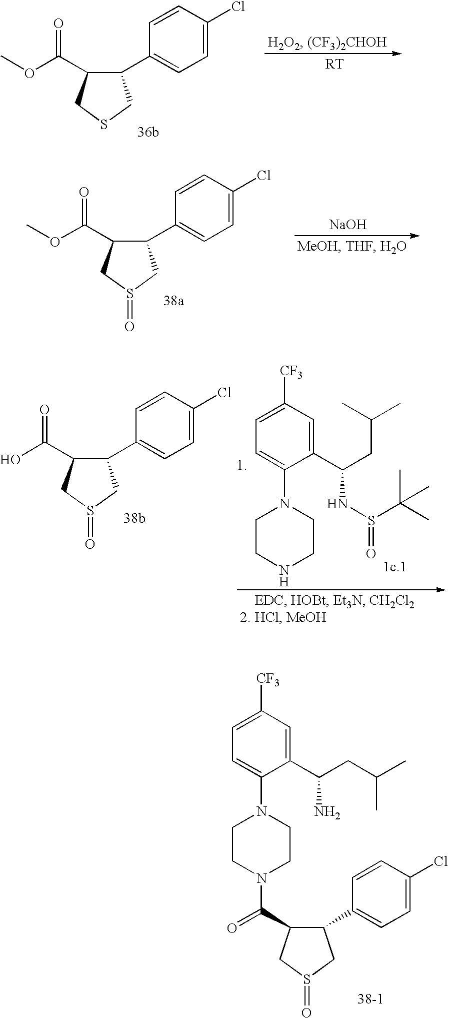

### Step 38A: Compound 38a

To a solution of 36b (500 mg, 1.95 mmol) in hexafluoroisopropanol (2.5 mL) was added H₂O₂ (31.3% aqueous solution, 0.44 mL) and the mixture was stirred forl h at room temperature. Saturated Na₂S₂O₃ (3 mL) was added to the reaction, and the fluorous layer was separated and concentrated. The product was purified by flash column chromatography (10% MeOH/CH₂Cl₂) to afford 357 mg (1.31 mmol) of 38a as a white solid in 67% yield.

### Step 38B: Compound 38b

The substrate 38a (350 mg, 1.29 mmol) was dissolved in H₂O/THF/MeOH (5 mL each) and NaOH (50% , 0.2 mL) was added to the solution. The mixture was stirred for 2 h at room temperature and then was concentrated at reduced pressure. The remaining solution was diluted with H₂O and extracted with Et₂O. The aqueous solution was acidified with 10% HCl then extracted with EtOAc twice to afford the acid 38b (299 mg, 1.16 mmol) as a white solid in 90% yield.

### Step 38C: Compound 38-1

To the mixture of 38b (0.20 mmol) and piperazine 1c.1 (52.3 mg, 0.25 mmol), was added EDC (HCl salt, 57 mg, 0.30 mmol), HOBt (41 g, 0.3 mmol) and Et₃N (0.11 mL, 0.8 mmol). The reaction was stirred at room temperature for 16 h, and then quenched with saturated NaHCO₃. The mixture was extracted with CH₂Cl₂, dried over Na₂SO₄, filtered, and concentrated. Half of the crude product (assuming quantitative yield from the previous step, 0.10 mmol) was dissolved in MeOH (1.0 mL), and treated with HCl (2.0 M in Et₂O, 0.075 mL) for 30 min. The solvent was evaporated and the final product was purified by preparative HPLC to afford 38-1 (TFA salt, 45.8 mg, 0.068 mmol). The overall yield was 68% over two steps.

By the above procedures, the compounds of the following Table 38 were prepared.

TABLE 38

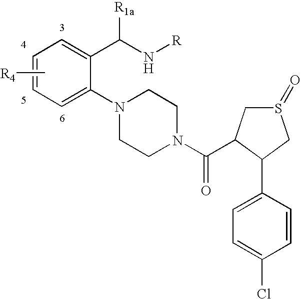

| Cpd | R₄ | R₁ₐ | R | MW | (MH⁺) |
|---|---|---|---|---|---|
| 38-1 | 4-Cl | i-Pr | H | 508.5 | 508 |
| 38-2 | 4-Me | i-Pr | H | 488.1 | 488 |
| 38-3 | 4-CF₃ | i-Pr | H | 542.1 | 542 |
| 38-4 | 4-CF₃ | i-Bu | —COCH₂CH₂NMe₂ | 655.2 | 655 |
| 38-5 | 4-Cl | i-Pr | —COCH₂CH₂NMe₂ | 607.6 | 607 |
| 38-6 | 4-Me | i-Pr | —COCH₂CH₂NMe₂ | 587.22 | 587 |
| 38-7 | 4-CF₃ | i-Pr | —COCH₂CH₂NMe₂ | 641.2 | 641 |

### Example 39

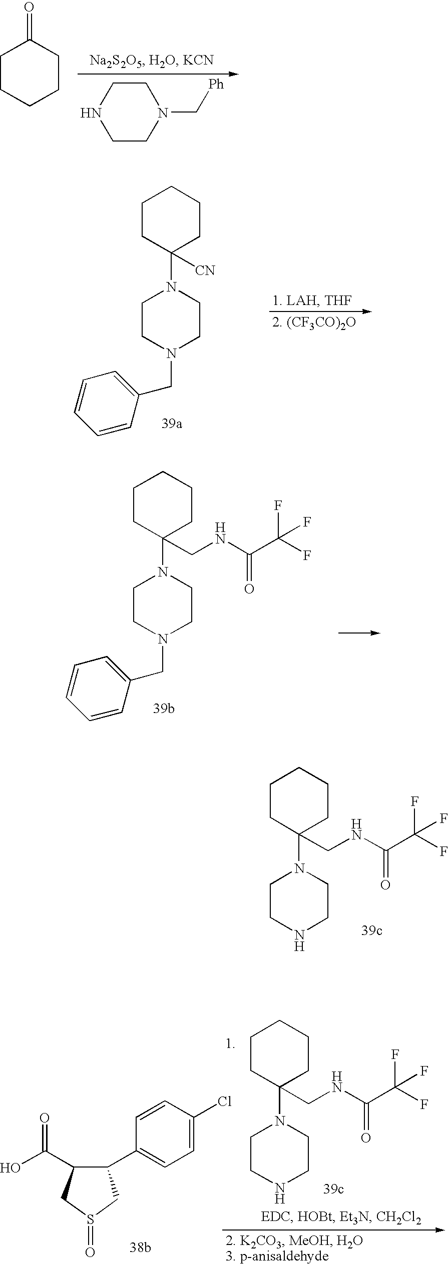

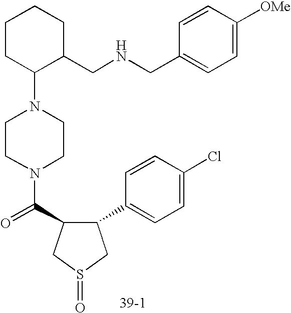

Step 39A: 1-(1-Cyanocyclohexyl)-4-benzylpiperazine 39a Cyclohexanone (7.3 mL, 70 mmol) was dissolved in water (140 mL) along with Na₂S₂O₅ (6.4 g, 35 mmol). The mixture was allowed to stir at room temperature for 1.5 hours then 1-benzylpiperazine (12.2 mL, 70 mmol) was added. The mixture was stirred for 2 hours and KCN (4.8 g, 74 mmol) was added to the reaction mix. The reaction mixture was then allowed to stir at room temperature overnight. The product was then extracted with dichloromethane (3×200 mL). The combined extracts were dried over anhydrous MgSO₄, filtered, and solvent was removed under vacuum. Compound 39a was obtained as a white solid in quantitative yield.

### Step 39B: 1-[1-(Trifluoroacetamidomethyl)cyclohexyl]-4-benzylpiperazine 39b

1-(1-Cyanocyclohexyl)-4-benzylpiperazine 39a (10 g, 35.3 mmol) was dissolved in ether (176 mL) and added dropwise to a mixture of LiAlH₄ (2.7 g, 71 mmol) in ether (353 mL) at room temperature. After the addition, the mixture was allowed to stir at room temperature for 0.5 hours. The reaction was then quenched by adding 2 mL of H₂O, followed by 1.5 mL of 20% NaOH, then 7 mL of H₂O. The reaction mixture was then filtered through celite and the residue was washed with ether. The ethereal mother liquor was dried over anhydrous MgSO₄ and solvent was removed under vacuum. The intermediate amine product was recovered in 94% yield without any further purification. This amine intermediate (9.5 g, 33 mmol) was then dissolved in dichloromethane (100 mL) along with Et₃N (4.8 mL, 34.7 mmol) and the reaction mixture was cooled to 0° C. To the reaction flask, trifluoroacetic anhydride (4.9 mL, 34.7 mmol) was added and the reaction was stirred at 0° C. for 10 minutes then at room temperature for 4 hours. Compound 39b was obtained as a clear oil (quantitative yield) after the reaction mixture was concentrated under vacuum. No further purification was needed.

### Step 39C: Compound 39c

1-[1-(Trifluoroacetamidomethyl)cyclohexyl]-4-benzylpiperazine 39b (13 g, 33 mmol) was dissolved in MeOH (192 mL) and the solution was degassed with nitrogen for 5 minutes. To the reaction flask, 10% by weight Pd on carbon (5 g) was added along with ammonium formate (6.2 g, 99 mmol). The mixture was allowed to stir at 65° C. for 2 hours. The reaction was then cooled to room temperature, filtered through celite, washed with degassed methanol, and solvent was removed under vacuum. The resulting residue was dissolved in dichloromethane (150 mL) and washed with sat. NaHCO₃ (3×150 mL) followed by washing with sat. NaCl solution (1×200 mL). The organic layer was then dried over anhydrous MgSO₄, filtered, and solvent was removed under vacuum. The deprotected piperazine 39c was obtained as a clear oil in 86% yield.

### Step 39D: Compound 39d

To the mixture of 38b (0.20 mmol) and piperazine 39c (73.3 mg, 0.25 mmol) in methylene chloride, was added EDC (HCl salt, 57 mg, 0.30 mmol), HOBt (41 mg, 0.3 mmol) and Et₃N (0.11 mL, 0.8 mmol). The mixture was stirred at room temperature for 16 h, and then quenched with saturated NaHCO₃. The product was extracted with CH₂Cl₂, dried over Na₂SO₄, filtered, and concentrated. The crude product was dissolved in 1.5 mL MeOH, 2 drops of H₂O, and K₂CO₃ (550 mg, 4.0 mmol) and heated at 100° C. in a pressure vessel for 2.5 h. After cooling, 10 mL H₂O was added and the product was extracted with CH₂Cl₂. The organic solution was dried over Na₂SO₄, filtered, concentrated, and dissolved in 1 mL MeOH. To half of the solution (assuming quantitative yield from the previous step, 0.10 mmol) was added p-anisaldehyde (0.037 mL, 0.3 mmol) and the mixture was stirred for 16 h. NaBH₄ (15 mg, 0.4 mmol) was added to the mixture and the stirring continued for 1 h. The solvent was evaporated and the remaining mixture was dissolved in CH₂Cl₂ and washed with saturated NaHCO₃. The organic solution was dried over Na₂SO₄, filtered, concentrated and purified by preparative HPLC to obtain 18.4 mg of 39-1 as the TFA salt (0.027 mmol). The total yield was 27% yield over 3 steps.

### Example 40

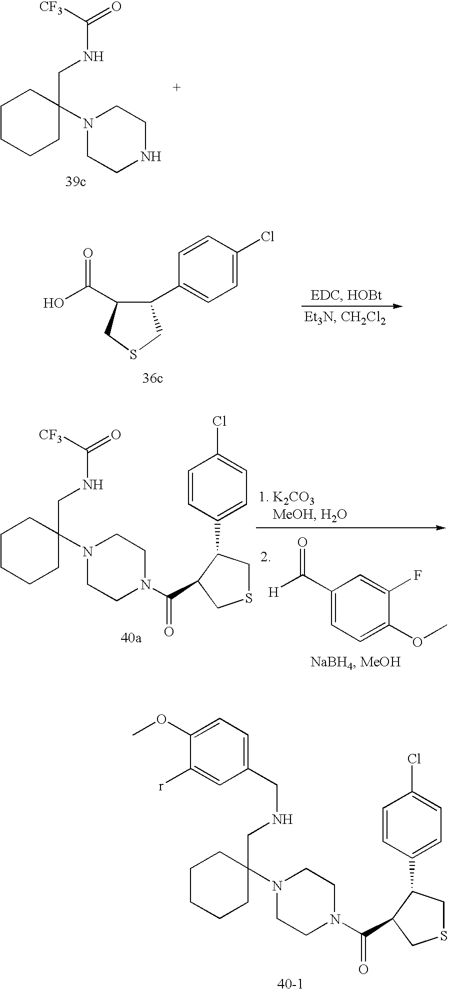

### Step 40A: Compound 40a

To the mixture of 36c (150 mg, 0.62 mmol) and piperazine 39c (191 mg, 0.65 mmol) in 3 mL CH₂Cl₂ was added EDC.HCl (178 mg, 0.93 mmol), HOBt (126 mg, 0.93 mmol) and Et₃N (0.13 mL, 0.93 mmol). The reaction mix was stirred at room temperature for 16 h, and was quenched with saturated NaHCO₃. The mixture was extracted with CH₂Cl₂, and the CH₂Cl₂ layer was dried over Na₂SO₄, filtered, and concentrated. Compound 40a (320 mg, 0.62 mmol) was obtained in quantitative yield and was used directly in the following steps.

### Step 40B: Compound 40-1

Compound 40a (158 mg, 0.30 mmol) was dissolved in 4.4 mL MeOH and 0.35 mL H₂O. To the solution was added 1.01 g K₂CO₃ (7.30 mmol). The reaction mix was heated to 60° C. for 8 h. After cooling, 3 mL H₂O was added and the mixture was extracted with CH₂Cl₂ twice. The organic solution was dried over Na₂SO₄, filtered, and concentrated to give 148 mg of material. Approximately 50 mg of this material was dissolved in 0.5 mL MeOH, and to this solution was added 3-fluoro-4-methoxybenzaldehyde (31 mg, 0.2 mmol). The mixture was stirred for 16 h and then NaBH₄ was added. After another 2 h, 0.75 mL saturated NaHCO₃ was added and the mixture was extracted with CH₂Cl₂ twice. The organic layer was concentrated and the residue was purified by HPLC to afford the TFA salt of 40-1 (13.3 mg, 0.019 mmol). The yield over 3 steps was 20% .

By the above procedures, the compounds of the following Table 40 were prepared.

TABLE 40

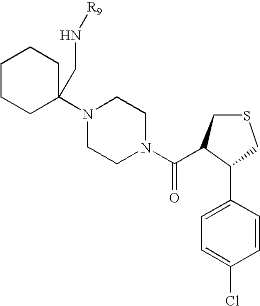

| Cpd | R₉ | MW | (MH⁺) |
|---|---|---|---|
| 40-1 | 3-F-4-MeOC₆H₄CH₂ | 560.2 | 560 |
| 40-2 | H | 422.0 | 422 |
| 40-3 | 4-MeOC₆H₄CH₂ | 542.2 | 542 |

### Example 41

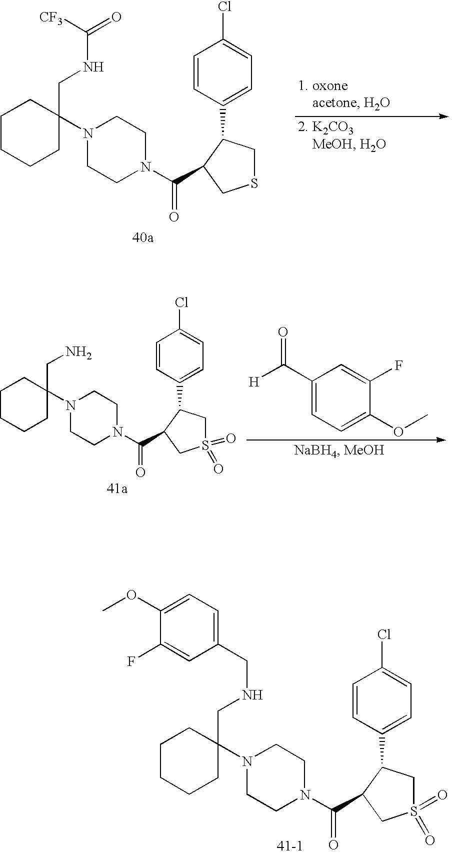

### Step 41A: Compound 41a

Oxone (614 mg, 1.0 mmol) in acetone/H₂O (1 mL each) was made basic with NaHCO₃ and 40a (160 mg, 0.31 mmol) was added to the mixture. The mix was stirred at room temperature for 2 h. Acetone was evaporated and the mixture was extracted with CH₂Cl₂. The organic layer was evaporated to give 160 mg of compound which was dissolved in 4.4 mL MeOH and 0.35 mL H₂O. To the solution was added 1.01 g K₂CO₃ (7.30 mmol). The mix was heated to 60° C. for 8 h. After cooling, 3 mL H₂O was added and the reaction mix was extracted with CH₂Cl₂ twice. The organic solution was dried over Na₂SO₄, filtered, and concentrated to give 41a whichwas used directly in the following step.

### Step 41B: Compound 41b

Compound 41a (50 mg, ˜0.1 mmol) was dissolved in 0.5 mL MeOH and 3-fluoro-4-methoxybenzaldehyde (31 mg, 0.2 mmol) was added. The mixture was stirred for 16 h and NaBH₄ was added to the reaction mix. After another 2 h, 0.75 mL saturated NaHCO₃ was added and the mixture was extracted with CH₂Cl₂ twice. The organic layer was evaporated and the residue was purified by HPLC to afford the TFA salt of 41-1 (3.8 mg, 0.005 mmol). The yield over 3 steps was 5% .

By the above procedures, the compounds of the following Table 41 were prepared.

TABLE 41

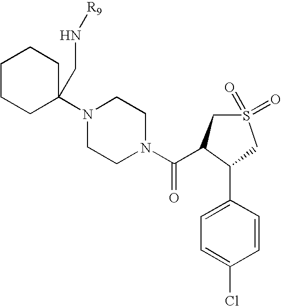

| Cp | R₉ | MW | (MH⁺) |
|---|---|---|---|
| 41-1 | 3-F-4-MeOC₆H₃CH₂ | 592.2 | 592 |
| 41-2 | H | 454.0 | 454 |
| 41-3 | 4-MeOC₆H₄CH₂ | 574.2 | 574 |
| 41-4 | 4-iPrOC₆H₄CH₂ | 602.2 | 602 |
| 41-5 | 4-FC₆H₄CH₂ | 562.1 | 562 |
| 41-6 | 3,4-CH₂O₂C₆H₃CH₂ | 588.2 | 588 |
| 41-7 | 1-Me-imidazolylCH₂ | 548.1 | 548 |
| 41-8 | 2-FuranylCH₂ | 534.1 | 534 |
| 41-9 | 5-PyrimidylCH₂ | 546.1 | 546 |

### Example 42

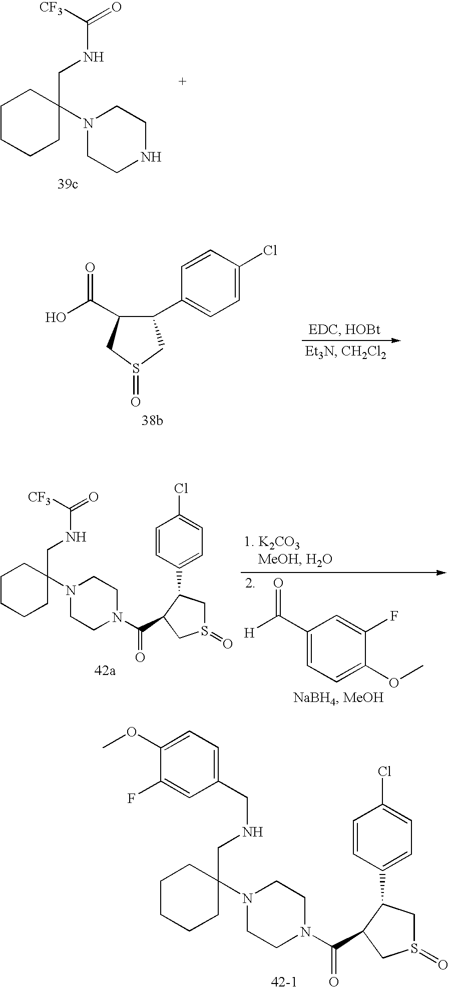

### Step 42A: Compound 42a

To the mixture of 38b (54 mg, 0.20 mmol) and piperazine 39c (440 mg, 0.3 mmol)) in 1 mL CH₂Cl₂ was added EDC.HCl (57 mg, 0.30 mmol), HOBt (41 mg, 0.30 mmol) and Et₃N (0.08 mL, 0.60 mmol). The reaction mixture was stirred at room temperature for 16 h, and was quenched with saturated NaHCO₃. The mixture was extracted with CH₂Cl₂, the organic layer dried over Na₂SO₄, filtered, and concentrated to give 42a which was used directly in the following steps.

### Step 42B: Compound 42-1

Compound 42a (˜0.20 mmol) was dissolved in 2.8 mL MeOH and 0.25 mL H₂O. To the solution was added 0.67 g K₂CO₃ (4.8 mmol). The reaction mixture was heated to 100° C. for 2 h. After cooling, 2 mL H₂O was added and the product was extracted with CH₂Cl₂ twice. The organic solution was dried over Na₂SO₄, filtered, and concentrated to a residue.

Half of the residue was dissolved in 0.5 mL MeOH, and 3-fluoro-4-methoxybenzaldehyde (31 mg, 0.2 mmol) was added. The mixture was stirred for 16 h and NaBH₄ was added to the reaction. After another 2 h, 0.75 mL saturated NaHCO₃ was added and the mix was extracted with CH₂Cl₂ twice. The organic layer was evaporated and the residue was purified by HPLC to afford the TFA salt of 42-1 (29.6 mg, 0.043 mmol). The yield over 3 steps was 43% .

By the above procedures, the compounds of the following Table 42 were prepared.

TABLE 42

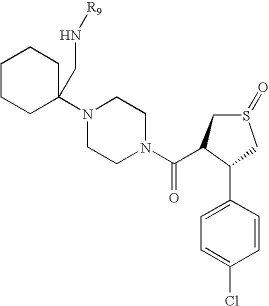

| Cpd | R₉ | MW | (MH⁺) |
|---|---|---|---|
| 42-1 | 3-F-4-MeOC₆H₄CH₂ | 576.2 | |
| 42-2 | H | 556.1 | 556 |
| 42-3 | 4-MeOC₆H₄CH₂ | 558.2 | 558 |

### Example 43

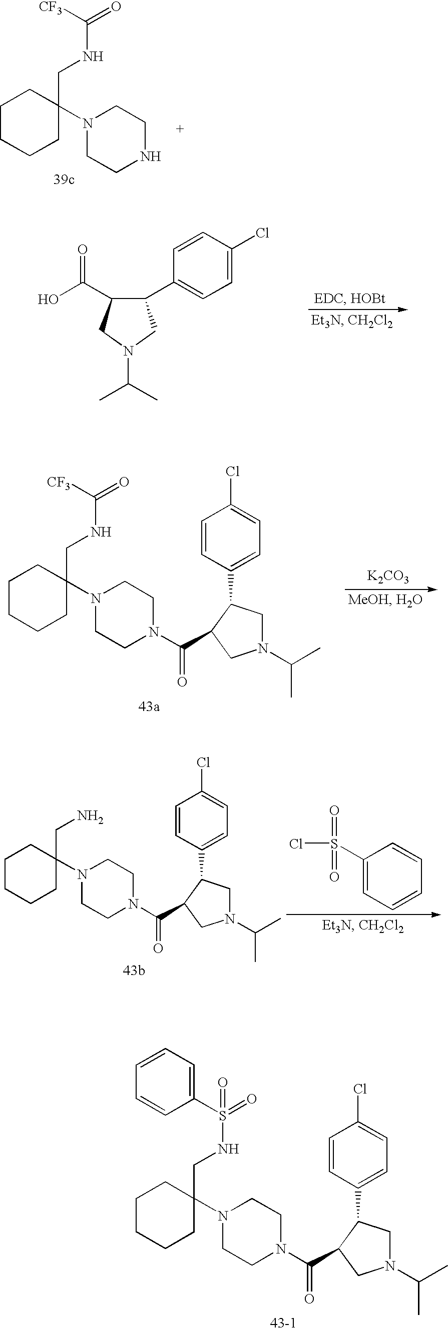

### Step, 43A: Compound 43a

To a mixture of 39c (1.64 g, 5.61 mmol) and trans-1-isopropyl-3-(4-chlorophenyl)pyrrolidine-4-carboxylic acid (1.50 g, 5.10 mmol) in 26 mL CH₂Cl₂ was added EDC.HCl (1.46 g, 7.65 mmol), NHOBt (1.03 g, 7.65 mmol) and Et₃N (1.35 mL, 10.2 mmol). The reaction mix was stirred at room temperature for 16 h, and was quenched with saturated NaHCO₃. The product was extracted with CH₂Cl₂, dried over Na₂SO₄, filtered, and concentrated. After purification by column chromatography, 43a (2.393 g, 5.33 mmol) was obtained in quantitative yield:

### Step 43B: Compound 43-1

Compound 43a (2.39 g, crude material, 5.33 mmol) was dissolved in 76 mL MeOH and 6 mL H₂O. To the solution was added 17.7 g K₂CO₃ (128 mmol). The reaction mix was heated to 65° C. for 16 h. After cooling, 50 mL H₂O was added and the reaction mixture was extracted with EtOAc (100 mL) twice. The organic solution was dried over Na₂SO₄, filtered, and concentrated to afford 43b 1.937 g (4.34 mmol). The yield was 85% over two steps.

### Step 43C: Compound 43-1

To the solution of 43b (30 mg, 0.067 mmol) in 0.5 mL CH₂Cl₂ was added phenyl sulfonyl chloride (59 mg, 0.1 mmol) and Et₃N (0.027 mL, 0.2 mmol). The mixture was stirred for 14 h and was quenched with saturated NaHCO₃. The mix was extracted with CH₂Cl₂ twice, dried over Na₂SO₄, filtered and concentrated. Purification by HPLC afforded the TFA salt of 43-1 (33.6 mg, 0.048 mmol) in 72% yield.

By the above procedures, the compounds of the following Table 43 were prepared.

TABLE 43

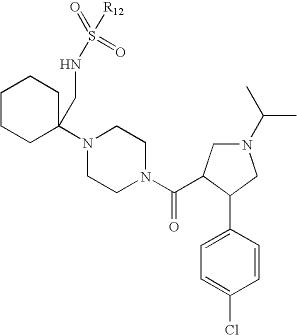

| Cpd | R₁₂ | MW | (MH⁺) |
|---|---|---|---|
| 43-1 | Ph | 587.2 | 587 |
| 43-2 | Et | 539.2 | 539 |
| 43-3 | Bn | 601.3 | 601 |
| 43-4 | 4-FBn | 619.2 | 619 |
| 43-5 | 4-FPh | 605.2 | 605 |
| 43-6 | 3,4-MeOPh | 647.3 | 647 |

### Example 44

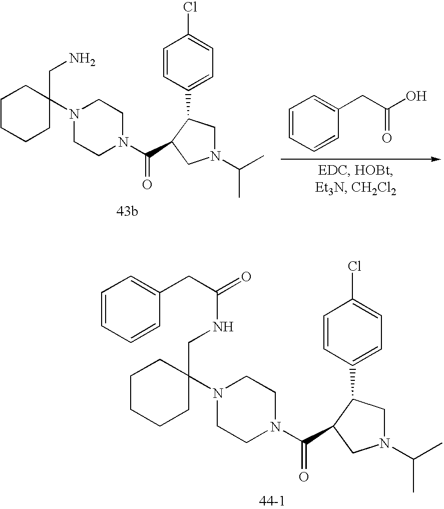

### Step 44A: Compound 44a

To the mixture of 43b (31 mg, 0.07 mmol) and phenylacetic acid (14 mg, 0.1 mmol) in 0.5 mL CH₂Cl₂ was added EDC.HCl (19 mg, 0.1 mmol), HOBt (14 mg, 0.1 mmol) and Et₃N (0.027 mL, 0.2 mmol). The reaction mixture was stirred at room temperature for 16 h, and was quenched with saturated NaHCO₃. The mixture was extracted with CH₂Cl₂, dried over Na₂SO₄, filtered, and concentrated. The residue was purified by HPLC to obtain the TFA salt of 44-1 (33 mg, 0.049 mmol) in 70% yield.

By the above procedures, the compounds of the following Table 44 were prepared.

TABLE 44

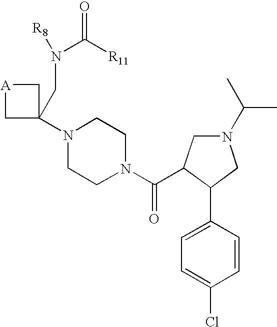

| Cpd. | A | R₁₁ | R₈ | MW | (MH⁺) |
|---|---|---|---|---|---|
| 44-1 | (CH₂)₃ | Bn | H | 565.2 | 565 |
| 44-2 | CH₂ | Ph | H | 523.1 | 523 |
| 44-3 | (CH₂)₂ | Me | H | 475.1 | 475 |
| 44-4 | (CH₂)₂ | Ph | H | 537.1 | 537 |
| 44-5 | (CH₂)₃ | 2-MeOBn | H | 595.2 | 595 |
| 44-6 | (CH₂)₃ | 4-MeOBn | H | 595.2 | 595 |
| 44-7 | (CH₂)₃ | 2-ClBn | H | 599.6 | 599 |
| 44-8 | (CH₂)₃ | 3-ClBn | H | 599.6 | 599 |
| 44-9 | (CH₂)₃ | 4-ClBn | H | 599.6 | 599 |
| 44-10 | (CH₂)₃ | 3-thienylCH₂ | H | 571.2 | 571 |
| 44-11 | (CH₂)₃ | 2-PyCH₂ | H | 566.2 | 566 |
| 44-12 | (CH₂)₃ | 3-PyCH₂ | H | 566.2 | 566 |
| 44-13 | (CH₂)₃ | 4-CF₃OBn | H | 649.2 | 649 |
| 44-14 | (CH₂)₃ | 1-Me-3-indolylCH₂ | H | 618.3 | 618 |
| 44-15 | (CH₂)₃ | 2-FBn | H | 583.2 | 583 |
| 44-16 | (CH₂)₃ | 3-FBn | H | 583.2 | 583 |
| 44-17 | (CH₂)₃ | 3-MeOBn | H | 595.2 | 595 |
| 44-18 | (CH₂)₃ | 2-thienylCH₂ | H | 571.2 | 571 |
| 44-19 | (CH₂)₃ | 4-MeOPh | H | 581.2 | 581 |
| 44-21 | (CH₂)₃ | Me | 4-MeOBn | 609.3 | 609 |
| 44-22 | (CH₂)₃ | Me | 4-PyCH₂ | 580.2 | 580 |
| 44-23 | CH₂OCH₂ | Me | H | 491.1 | 491 |
| 44-24 | CH₂OCH₂ | Ph | H | 553.1 | 553 |

### Example 45

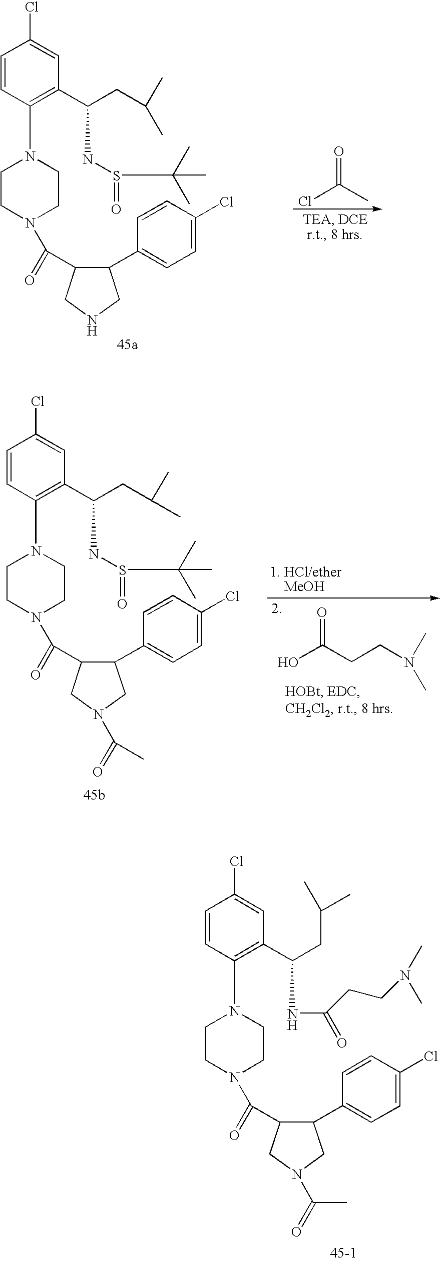

### Step 45A: Compound 45b

In a 4 dram reaction vial, pyrrolidine intermediate 45a (0.059 g, 0.10 mmol) was dissolved in dichloroethane (1 mL) along with acetyl chloride (0.007 mL, 0.10 mmol) and triethylamine (0.014 mL, 0.10 mmol). The reaction mixture was capped and stirred for 8 hours at room temperature. The reaction mixture was diluted with dichloromethane (1 mL) and washed with saturated NaHCO₃ solution (1 mL). The organic layer was collected and solvent was reduced under a stream of nitrogen to afford 45b in quantitative yield 0.063 g, 0.10 mmol). This intermediate was used for the next step without further purification.

### Step 45B: Compound 45-1

In a capped vial, the sulfinamide 45b (0.063 g, 0.10 mmol) was dissolved in methanol (1 mL) and then treated with 2M HCl in diethyl ether (0.20 mmol). The reaction mixture was capped and stirred for 20 minutes at room temperature. The mixture was then diluted with dichloromethane (1 mL) and neutralized with saturated NaHCO₃. The organic layer was collected, transferred to a 4 dram vial, and then solvent was reduced by a stream of nitrogen to afford an intermediate which was dissolved in dichloromethane (1 mL) along with dimethylaminopropionic acid (0.015 g, 0.10 mmol) and HOBt (0.016 g, 0.12 mmol). The reaction mixture was capped and stirred for 15 minutes at room temperature before adding EDC (0.023 g, 0.12 mmol). The reaction mixture was stirred for 8 hours, diluted with dichloromethane (1 mL) and washed with saturated NaHCO₃ (1 mL). The organic layer was collected and reduced under a stream of nitrogen to give a residue which was purified by prep HPLC to give 45-1 (0.019 g, 31% ). LCMS (tᵣ, 4.989) 630 (M+H)

By the above procedures, the compounds of the following Table 45 were prepared.

TABLE 45

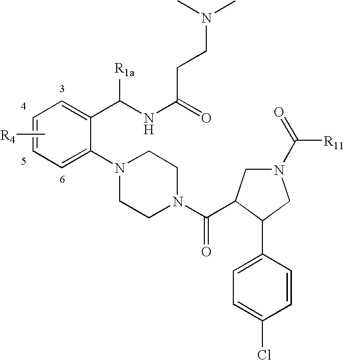

| Cpd | R₄ | R₁ₐ | R₁₁ | MW | (MH⁺) | |
|---|---|---|---|---|---|---|
| 45-1 | 4-Cl | iBu | Me | 630.7 | 630 | 4.989 |
| 45-2 | 4-Cl | iBu | Ph | 692.7 | 692 | 5.463 |
| 45-3 | 6-F | iPr | Ph | 662.2 | 662 | 6.804 |
| 45-4 | 6-F | iPr | Me | 600.2 | 600 | 4.761 |
| 45-5 | 4-Cl | iBu | Et | 644.7 | 644 | 5.148 |
| 45-6 | 4-Cl | iBu | Pr | 658.7 | 658 | 5.312 |
| 45-7 | 4-Cl | iBu | cBu | 670.7 | 670 | 5.362 |
| 45-8 | 6-F | iPr | Et | 614.2 | 614 | 5.143 |
| 45-9 | 6-F | iPr | Pr | 628.2 | 628 | 5.186 |
| 45-10 | 6-F | iPr | cBu | 640.2 | 640 | 5.108 |
| 45-11 | 6-F | iPr | tBu | 642.3 | 642 | 5.165 |
| 45-12 | 4-Me | iPr | Me | 596.2 | 596 | 1.596 |
| 45-13 | 4-Me | iPr | Et | 610.2 | 610 | 1.535 |

### Example 46

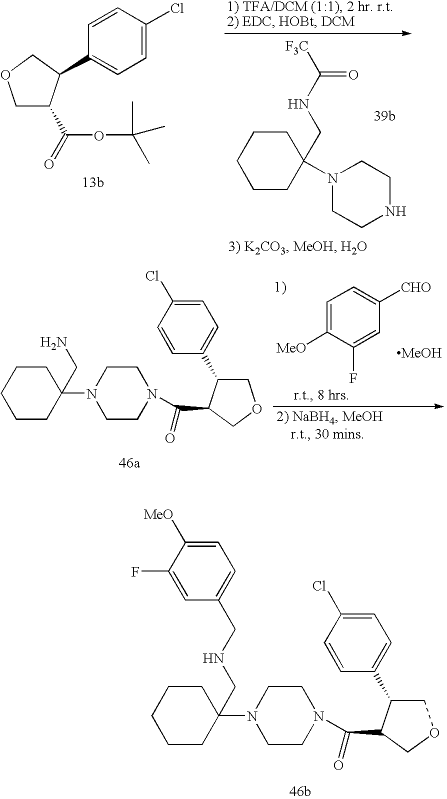

### Step 46A: Compound 46a

Tetrahydrofuran t-butyl ester 13b (382 mg, 1.35 mmol) was dissolved in 1:1 TFA/DCM (4 mL) and stirred at room temperature for 2 hours. Solvent and excess TFA was removed in vacuo to give the desired tetrahydrofuran acid in quantitative yield. A portion of the tetrahydrofuran acid intermediate (136 mg, 0.6 mmol) was dissolved in DCM (6 mL) along with HOBt (81 mg, 0.6 mmol), cyclohexyl piperazine 39b (176 mg, 0.6 mmol), and triethylamine (84 uL,0.6 mmol). The reaction mixture was allowed to stir at room temperature for 10 minutes then EDC (115 mg, 0.6 mmol) was added. The reaction mixture stirred at room temperature for an additional 8 hours. After 8 hours, the reaction mixture was washed with saturated NaHCO₃ (3×10 mL) and saturated NaCl (10 mL). The organic layer was collected, dried over anhydrous MgSO₄, filtered, and evaporated to dryness under vacuum. The residue was dissolved in methanol (8.6 mL) along with water (0.7 mL, 38.8 mmol) and potassium carbonate (2 g, 14.5 mmol). The reaction mixture was allowed to stir at 65° C. for 3 hours. The reaction was cooled to room temperature, filtered, and diluted with ether (30 mL). The organic layer was washed with water (2×10 mL) and saturated NaCl (10 mL). The organic phase was dried over anhydrous Na₂SO₄, filtered, and solvent was removed under vacuum to give 46a which was used in the next step without further purification.

### Step 46B: Compound 46-1

In a 4 mL reaction vial, tetrahydrofuran cyclohexylamine 46a (36.5 mg, 0.09 mmol) was dissolved in methanol (1 mL) along with 3-fluoro-4-methoxy-benzaldehyde (13 mg, 0.085 mmol). The reaction mix was allowed to stir at room temperature for 8 hours. NaBH₄ (5.5 mg, 0.14 mmol) was added and the mixture was allowed to stir at room temperature for an additional 30 minutes. The reaction mixture was quenched with 1 mL of 1N NaOH and extracted with ether. The ethereal extract was then concentrated under a stream of nitrogen and the residue was purified by preparative HPLC. Compound 46-1 was recovered as the TFA salt in 29% overall yield from compound 46a. MS: calc. for C₃₀H₃₉ClFN₃O₃: 543.3; Found: 543.8 (M+H); retention time: 5.827 minutes

By the above procedures, the compounds of the following Table 45 were prepared.

TABLE 46

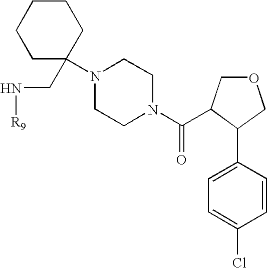

| Cpd | R₉ | MW | (MH⁺) |
|---|---|---|---|
| 46-1 | 3-F-4-MeOBn | 544.1 | 543.8 |
| 46-2 | Bn | 496.1 | 495.8 |
| 46-3 | 4-PyCH₂ | 497.1 | 496.8 |
| 46-4 | 4-MeOBn | 526.1 | 525.8 |
| 46-5 | 2-FBn | 514.1 | 513.8 |

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

It will be appreciated that, although specific embodiments of the invention have been described herein for purposes of illustration, various modifications may be made without departing from the spirit and scope of the invention. Accordingly, the invention is not limited except as by the appended claims.

1. A compound having the following structure: 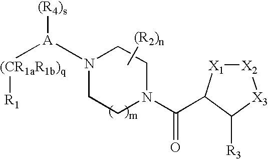
or a pharmaceutically acceptable salt, ester, solvate, stereoisomer, or prodrug thereof,
wherein:
A is a C₅₋₇cycloalkyl, aryl, or heteroaryl;
X₁ is —CR₅R₆—, —NR₇—, —O—, or —C(═O)—;
X₂ and X₃ are the same or different and independently —CR₅R₆—, —NR₈—, —O—, or —C(═O)—;
or X₁ taken together with X₂ is —N═C(R₅)— or —C(R₅)═N—;
or X₂ taken together with X₃ is —N═C(R₅)— or —C(R₅)═N—;
R₁ is —(Y—Y₂)—NR₉R₁₀, —NR₈C(═O)R₁₁, —NR₈S(O)ₚR₁₂, —NR₈C(═O)R₁₃, imidazolyl, triazolyl, oxazolyl, or thiazolyl;
Y₁ is a direct bond, —O—, —S——NR₈—, —C(═O)—, —C(═O)O—, —OC(═O)—, —NR₈C(═O)O—, —NR₈C(═O)—, —C(═O)NR₈—, —NR₈S(═O)ₚ—, —S(═O)ₚ—, —S(═O)ₚNR₈—, or —NR₈C(═O)NR₈—;
Y₂ is —(CR_{1c}R_{1d})ᵣ—;
R₁ₐ, R_{1b}, R_{1c}, and R_{1d} are at each occurrence the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
R₂ is at each occurrence the same or different and independently alkyl or substituted alkyl;
R₃ is aryl, substituted aryl, heteroaryl or substituted heteroaryl;
R₄ is at each occurrence the same or difference and independently hydroxy, halogen, cyano, nitro, alkyl, haloalkyl, substituted alkyl, aryl, substituted aryl, heterocycle, or substituted heterocycle;
R₅ and R₆ are the same or different and at each occurrence independently hydrogen, hydroxy, halogen, cyano, nitro, NR₉R₁₀, alkyl, substituted alkyl, aryl, substituted aryl, heterocycle, or substituted heterocycle;
R₇ is hydrogen, alkyl, substituted alkyl, —C(═O)R₁₁, or —SO₂R₁₂;
R₈ is at each occurrence the same or different and independently hydrogen, alkyl, substituted alkyl, heterocycle, substituted heterocycle, arylalkyl, substituted arylalkyl, heterocyclealkyl, substituted heterocyclealkyl, —C(═O)R₁₁, or —SO₂R₁₂;
R₉ and R₁₀ are the same or different and at each occurrence independently hydrogen, alkyl, substituted alkyl, aryl, substituted aryl, arylalkyl, substituted arylalkyl, heterocycle, substituted heterocycle, heterocyclealkyl, or substituted heterocyclealkyl,
or R₉ and R₁₀ taken together with the nitrogen atom to which they are attached form a heterocyclic ring or a substituted heterocyclic ring;
R₁₁, R₁₂ and R₁₃ are the same or different and independently hydrogen, alkyl, substituted alkyl, heterocycle, substituted heterocycle, aryl, substituted aryl, heterocyclealkyl, substituted heterocyclealkyl, arylalkyl or substituted arylalkyl;
m,p and s are independently 0, 1 or 2; and
n, q and r are independently 0, 1, 2, 3 or 4.

2. The compound of claim 1 wherein A is C₅₋₇cycloalkyl.

3. The compound of claim 1 wherein A is aryl.

4. The compound of claim 3 wherein A is phenyl.

5. The compound of claim 1 wherein A is heteroaryl.

6. The compound of claim 1 wherein q is 1 or 2.

7. The compound of claim 1 wherein R₃ is aryl or substituted aryl.

8. The compound of claim 1 wherein R₃ is heteroaryl or substituted heteroaryl.

9. The compound of claim 1 wherein R₁ is —Y₁—Y₂—NR₉R₁₀.

10. The compound of claim 1 wherein R₁ is —NR₈C(═O)R₁₁, —NR₈S(O)ₚR₁₂, imidazolyl, triazolyl, oxazolyl, or thiazolyl.

11. The compound of claim 1 wherein X₁, X₂ and X₃, taken together as X₁—X₂—X₃, is —(CR₅R₆)₃—, —O—CR₅R₆—CR₅R₆—, —CR₅R₆—O—CR₅R₆—, —CR₅R₆—CR₅R₆—O—, —O—C(═O)—CR₅R₆—, —CR₅R₆—C(═O)—O—, —NR₇—CR₅R₆—CR₅R₆—, —CR₅R₆—NR₈—CR₅R₆—, —CR₅R₆—CR₅R₆—NR₈—, —NR₇—C(═O)—CR₅R₆—CR₅R₆—C(═O)—NR₈—, or —O—NR₈—CR₅R₆.

12. The compound of claim 1 wherein X₁, X₂ and X₃, taken together as X₁—X₂—X₃, is —CR₅R₆—O—NR₈—, —O—N═CR₅—, —NR₇—NR₈—CR₅R₆—, —CR₅R₆—NR₈—NR₈—, —NR₇—N═CR₅—, —O—CR₅R₆—NR₈—, —O—CR₅R₆—O—, —NR₇—C(═O)—O—, —NR₇—C(═O)—NR₈—, —N═CR₅—O—, —N=CR₅—NR₈— or —NR₇—O—CR₅R₆—, —CR₅R₆—NR₈—C(O)—, —O—CR₅═N—, —O—C(O)—NR₈—, —CR₅R₆—NR₈—O—, or —CR₅═N—O—.

13. A compound according to claim 1, wherein:
X₁ and X₃ are each CR₅R₆;
R₅ and R₆ are each H;
X₂ is N—R₈;
m is 1; and
n is 0.

14. A comound according to claim 13, wherein R₈ is arylalkyl or heterocycle.

15. A compound according to claim 14, wherein R₈ is tetrahydro-4-pyranyl or benzyl.

16. A compound according to claim 15, wherein R₃ is phenyl or substituted phenyl.

17. A compound according to claim 16, wherein A is aryl.

18. A compound according to claim 17, wherein A is phenyl.

19. A compound according to claim 18, wherein R₄ is alkyl or halogen, and s is 0.

20. A compound according to claim 19, wherein R₄ is methyl or fluoro.

21. A compound according to claim 20, wherein q is 1, and one of R₁ₐ and R_{1b} is hydrogen and the other is isopropyl.

22. A compound according to claim 21, wherein R₁ is NR₈C(O)R₁₁.

23. A compound according to claim 22, wherein R₈ is hydrogen and R₁₁ is —CH₂CH₂N(CH₃)₂.

24. A compound according to claim 21, wherein R₁ is —(Y₁Y₂)—NR₉R₁₀.

25. A compound according to claim 24, wherein Y₁ is a bond, r is 0, and R₉ and R₁₀ are each hydrogen.

26. A pharmaceutical composition comprising a compound according to any one of claims 1, 4, 7, 9, 10, 13, 19, 23 and 25 and a pharmaceutically acceptable carrier or diluent.

27. A method for treating a patient having a disorder associated with the activity of a melanocortin receptor, comprising administering to the patient a pharmaceutical composition comprising a pharmaceutically effective amount of a compound according to claim 1 and a pharmaceutically acceptable carrier or diluent.

28. The method of claim 27 wherein the melanocortin receptor is melanocortin 3 receptor.

29. The method of claim 27 wherein the melanocortin receptor is melanocortin 4 receptor.

30. The method of claim 27 wherein the compound is an antagonist of the melanocortin receptor.

31. The method of claim 27 wherein the compound is an agonist of the melanocortin receptor.

32. The method of claim 27 wherein the disorder is an eating disorder.

33. The method of claim 32 wherein the eating disorder is cachexia.

34. The method of claim 27 wherein the disorder is a sexual dysfunction.

35. The method of claim 34 where the sexual dysfunction is erectile dysfunction.

36. The method of claim 27 wherein the disorder is a skin disorder.

37. The method of claim 27 where the disorder is chronic pain.

38. The method of claim 27 where the disorder is anxiety or depression.

39. The method of claim 27 wherein the disorder is obesity.
